(19) United States
(12) Patent Application Publication
(10) Pub. No.: US 2013/0225609 A1
(43) Pub. Date: Aug. 29, 2013

(54) COMBINATIONS OF MEDICAMENTS, CONTAINING PDE4-INHIBITORS AND EP4-RECEPTOR-ANTAGONISTS

(71) Applicant: Peter Nickolaus, Warthausen (DE)

(72) Inventor: Peter Nickolaus, Warthausen (DE)

(73) Assignee: BOEHRINGER INGELHEIM INTERNATIONAL GMBH, Ingelheim am Rhein (DE)

(21) Appl. No.: 13/639,629

(22) PCT Filed: Apr. 1, 2011
(86) PCT No.: PCT/EP2011/055074

§ 371 Date: May 16, 2013

(30) Foreign Application Priority Data
Apr. 8, 2010 (EP) 10159390.3

(51) Int. Cl. A61K 31/519; A61K 31/4196; A61K 31/4035; A61K 31/437; A61K 31/443; A61K 31/4745; A61K 31/192

(52) U.S. Cl. CPC A61K 31/519; A61K 31/4745; A61K 31/4196; A61K 31/192; A61K 31/437; A61K 31/443; A61K 31/4035
USPC 514/260.1

(57) ABSTRACT

The present invention relates to new medicament combinations which contain in addition to one or more PDE4-inhibitors (1) at least one EP4 receptor antagonist (2), as well as the use thereof for the treatment of preferably respiratory complaints such as particularly COPD, chronic sinusitis and asthma.

The invention relates in particular to those medicament combinations which contain, in addition to one or more, preferably one, PDE4 inhibitor of general formula 1

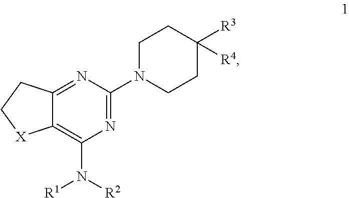

wherein X is SO or SO₂, but preferably SO, and wherein
R¹, R², R³ and R⁴ have the meanings given in claim 1,
at least one EP4 receptor antagonist (2), the preparation thereof and the use thereof for the treatment of respiratory complaints.

The present invention relates to new medicament combinations which contain in addition to one or more PDE4-inhibitors (1) at least one EP4 receptor antagonist (2), processes for preparing them and their use for the treatment of in particular respiratory complaints such as for example COPD, chronic sinusitis and asthma.

The invention relates in particular to those medicament combinations which comprise, in addition to one or more, preferably one, PDE4-inhibitor of general formula 1

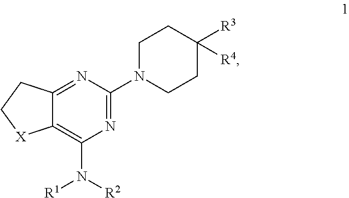

wherein
X is SO or SO₂, but preferably SO, and wherein R¹, R², R³ and R⁴ have the meanings given in claim 1,
at least one EP4 receptor antagonist (2), the preparation thereof and the use thereof for the treatment of respiratory complaints.

### PRIOR ART

WO2009050248 discloses piperidino-dihydrothienopyrimidines of formula 1 as PDE4-inhibitors, the preparation thereof as well as the use thereof for the treatment of respiratory complaints.

It is also known that many “1st generation” PDE4-inhibitors such as for example rolipram lead to undesirable side effects. Consequently, it was an objective of the present invention to provide a medicament or a medicament combination containing a PDE4 inhibitor which has a low side-effect profile. Surprisingly it has been found that an EP4 receptor antagonist that is administered simultaneously with a PDE4 inhibitor or a few hours (at most 6 hours) before or after a PDE4 inhibitor, greatly reduces the typical side effects of a PDE4 inhibitor, without having any appreciable side effects in long-term therapy.

EP4-receptor-antagonists such as for example [N-{[4-(5,9-diethoxy-6-oxo-6,8-dihydro-7H-pyrrolo[3,4-g]quinolin-7yl)-3-methylbenzyl]sulphonyl}-2-(2-methoxyphenypacetamide] (also known as MF498) were indeed already known for example from Clark et al; The Journal of Pharmacology and Experimental Therapeutics; Vol. 325, No. 2; pages 425-434, but it was not known that typical PDE4-mediated side effects are significantly reduced by EP4-receptor-antagonists of this kind.

The present invention therefore relates to a novel medicament combination which includes at least one EP4 receptor antagonist (2) in addition to one or more PDE4-inhibitors (1). The present invention preferably relates to those medicament combinations which contain in addition to one or more PDE4-inhibitors (1) at least one EP4 receptor antagonist (2) and wherein at least one EP4-receptor-antagonist (2) is an EP4-specific antagonist.

A preferred embodiment of the present invention relates to one of the above mentioned medicament combinations, wherein the at least one EP4 receptor antagonist is selected from among
[N-{[4-(5,9-diethoxy-6-oxo-6,8-dihydro-7H-pyrrolo[3,4-g]quinolin-7-yl)-3-methylbenzyl]sulphonyl}-2-(2-methoxyphenyl)acetamide] (2.1);
5-butyl-2,4-dihydro-4-[[2′4[N-(3-methyl-2-thiophene-carbonyl)sulphamoyl]biphenyl-4-yl]methyl]-2-[(2-trifluoromethyl)phenyl]-1,2,4-triazol-3-one (2.2);
(4-{(1S)-1-[({5-chloro-2-[(4-fluorophenyl)oxy]phenyl}carbonyl)amino]ethyl}benzoic acid (2.3);
N-[({2-[4-(2-ethyl-4,6-dimethyl-1H-imidazo[4,5-c]pyridin-1-yl)phenyl]ethyl}amino)carbonyl]-4-methylbenzene sulphonamide (2.4);
4-[[4-(5-methoxy-2-pyridinyl)phenoxy]methyl]-5-methyl-N-[(2-methylphenyl)sulphonyl]-2-furancarboxamide (2.5);
methyl 11 alpha, 15alpha-dihydroxy-16-(3-methoxymethylphenyl)-9-oxo-17,18,19,20-tetranor-5-thia-13(E)prostanoate (2.6);
4-cyano-2-[[2-(4-fluoro-1-naphthalenyl)-1-oxopropyl]amino]-benzenebutanoic acid (2.7) and
N-{2-[4-(4,9-diethoxy-1-oxo-1,3-dihydro-2H-benzo[f]isoindol-2-yl)phenyl]acetyl}benzene sulphonamide (2.8).

A particularly preferred embodiment of the present invention relates to one of the above mentioned medicament combinations, which comprises at least one EP4 receptor antagonist (2), in addition to one or more, preferably one, PDE4 inhibitor (1) of general formula 1

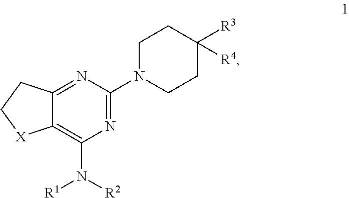

wherein
X denotes SO or SO₂,
R¹ denotes H, C₁₋₆-alkyl,
R² is H or a group selected from among C₁₋₁₀-alkyl and C₂₋₆-alkenyl, which may optionally be substituted by one or more groups selected from halogen and C₁₋₃-fluoroalkyl or which is optionally substituted by one or more groups selected from among OR^{2.1}, COOR^{2.1}, CONR^{2.2}R^{2.3}, SR^{2.1}, SO—R^{2.1}, SO₂—R^{2.1}, C₆₋₁₀-aryl, het, hetaryl, a mono- or bicyclic C₃₋₁₀-cycloalkyl, CH₂—NR^{2.2}R^{2.3} and NR^{2.2}R^{2.3}, which in turn may optionally be substituted by one or more groups selected from among OH, halogen, OR^{2.1}, oxo, CF₃, CHF₂, CH₂F, C₁₋₆-alkyl, C₁₋₆-alkanol, C₆₋₁₀-aryl, COOR^{2.1}, CH₂—NR^{2.2}R^{2.3} and NR^{2.2}R^{2.3},
   wherein
het is a three- to eleven-membered, mono- or bicyclic, saturated or partly saturated, optionally annellated or optionally bridged heterocyclic group, which contains 1, 2, 3 or 4 heteroatoms selected independently of one another from among N, S or O, and wherein
hetaryl is a five- to ten-membered, mono- or bicyclic, optionally annellated heteroaryl, which contains 1, 2, 3 or 4 heteroatoms selected independently of one another from among N, S or O,
   and wherein
cycloalkyl may be saturated or partly saturated,
wherein R^{2.1} is H or a group selected from among C₁₋₆-alkyl, C₁₋₆-alkanol, C₁₋₃-haloalkyl, mono- or bicyclic, —C₃₋₁₀-cycloalkyl, C₆₋₁₀-aryl-C₁₋₆-alkylene, hetaryl-C₁₋₆-alkylene, het-C₁₋₆-alkylene, C₃₋₁₀-cycloalkyl-C₁₋₆-alkylene, a mono- or bicyclic C₆₋₁₀-aryl, heteroaryl and a -het,
   which may optionally be substituted by one or more groups selected from among OH, O—(C₁₋₃-alkyl), halogen, C₁₋₆-alkyl and C₆₋₁₀-aryl,
wherein R^{2.2} and R^{2.3} independently of one another denote H or a group selected from among C₁₋₆-alkyl, mono- or bicyclic C₃₋₁₀cycloalkyl, C₆₋₁₀-aryl-C₁₋₆-alkylene, hetaryl-C₁₋₆-alkylene, mono- or bicyclic C₆₋₁₀-aryl, het, hetaryl, CO—NH₂, CO—NHCH₃, —CO—N(CH₃)₂, SO₂—(C₁-C₂-alkyl), CO—R^{2.1} and COOR^{2.1},
   which may optionally be substituted by one or more groups selected from among OH, halogen, C₁₋₆-alkyl, C₆₋₁₀-aryl and COOR^{2.1},
      or
R² denotes a mono- or polycyclic C₃₋₁₀ cycloalkyl, which may optionally be singly or multiply bridged by C₁₋₃-alkyl groups and which may optionally be substituted by a group selected from among branched or unbranched C₁₋₆-alkanol, C₁₋₃-fluoroalkyl, C₁₋₃-alkylene-OR^{2.1}, OR^{2.1}, COOR^{2.1}, —SO₂—NR^{2.2}R^{2.3}, het, —NH—CO—O—(C₁₋₆-alkyl), —NH—CO—(C₁₋₆-alkyl), —NH—CO—O—(C₆₋₁₀-aryl), —NH—CO—(C₆₋₁₀-aryl), —NH—CO—O-hetaryl, —NH—CO-hetaryl, —NH—CO—O—(C₁₋₃-alkylene)-(C₆₋₁₀-aryl), —NH—CO—(C₁₋₃-alkylene)-(C₆₋₁₀-aryl), —N(C₁₋₃-alkyl)-CO—(C₁₋₆-alkyl), —N(C₁₋₃-alkyl)-CO—O—(C₆₋₁₀-aryl), —N(C₁₋₃-alkyl)-CO—(C₆₋₁₀-aryl), —N(C₁₋₃-alkyl)-CO—O-hetaryl, —N(C₁₋₃-alkyl)-CO-hetaryl, —N(C₁₋₃-alkyl)-CO—O—(C₁₋₃-alkylene)-(C₆₋₁₀-aryl), —N(C₁₋₃-alkyl)-CO—(C₁₋₃-alkylene)-(C₆₋₁₀-aryl), C₆₋₁₀-aryl, C₁₋₆-alkyl, C₆₋₁₀-aryl-C₁₋₆-alkylene, hetaryl-C₁₋₆-alkylene, mono- or bicyclic C₃₋₁₀ cycloalkyl and NR^{2.2}R^{2.3},
   which may optionally be substituted by one or more groups selected from among OH, OR^{2.1}, oxo, halogen, CF₃, CHF₂, CH₂F, C₁₋₆-alkyl, C₆₋₁₀-aryl and NR^{2.2}R^{2.3},
      or
R² denotes a mono- or polycyclic C₆₋₁₀-aryl, which may optionally be substituted by OH, SH or halogen or by one or more groups selected from among OR^{2.1}, COOR^{2.1}, NR^{2.2}R^{2.3}, CH₂—NR^{2.2}R^{2.3}, C₃₋₁₀-cycloalkyl, het, C₁₋₆-alkyl, C₁₋₃-fluoroalkyl, CF₃, CHF₂, CH₂F, C₆₋₁₀-aryl-C₁₋₆-alkylene, het-C₁₋₆-alkylene, hetaryl-C₁₋₆-alkylene, C₆₋₁₀-aryl, SO₂—CH₃, SO₂—CH₂CH₃ and SO₂—NR^{2.2}R^{2.3},
   which in turn may optionally be substituted by one or more or several groups selected from among OH, OR^{2.1}, CF₃, CHF₂, CH₂F, oxo, halogen, CF₃, CHF₂, CH₂F, C₁₋₆-alkyl, C₆₋₁₀-aryl and NR^{2.2}R^{2.3},
      or
R² denotes a group selected from among het and hetaryl, which may optionally be substituted by one or more groups selected from among halogen, OH, oxo, CF₃, CHF₂ and CH₂F or by one or more groups selected from among OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, mono- or bicyclic C₃₋₁₀-cycloalkyl, C₆₋₁₀-aryl, C₁₋₆-alkyl, C₆₋₁₀-aryl-C₁₋₆-alkylene, hetaryl-C₁₋₆-alkylene, het, hetaryl, C₁₋₃-alkylene-OR^{2.1} and NR^{2.2}R^{2.3},
   which may optionally be substituted by one or more groups selected from among OH, OR^{2.1}, oxo, halogen, CF₃, CHF₂, CH₂F, C₁₋₆-alkyl, C₆₋₁₀-aryl and NR^{2.2}R^{2.3} may be substituted,
      or wherein
NR¹R² together denotes a heterocyclic four- to seven-membered ring which may optionally be bridged, which contains 1, 2 or 3 heteroatoms selected from among N, O and S and which may optionally be substituted by one or more groups selected from among OH, OR^{2.1}, C₁₋₃-alkylene-O^{R.1}, oxo, halogen, C₁₋₆-alkyl, C₆₋₁₀-aryl, COOR^{2.1}, CH₂—NR^{2.2}—COO—R^{2.1}, CH₂—NR^{2.2}—CO—R^{2.1}, CH₂—NR^{2.2}—CO—CH₂—NR^{2.2}R^{2.3}, CH₂—NR^{2.2}—SO₂—C₁₋₃-alkyl, CH₂—NR^{2.2}—SO₂—NR^{2.2}R^{2.3}, CH₂—NR^{2.2}—CO—NR^{2.2}R^{2.3}, CO—NR^{2.2}R^{2.3}, CH₂—NR^{2.2}R^{2.3} and NR^{2.2}R^{2.3},
   and wherein
R³ is a C₆₋₁₀-aryl,
   which is optionally substituted in the ortho, para or meta position with one, two or three groups independently of one another selected from among fluorine, chlorine, bromine, hydroxy, CN, C₁₋₆-alkyl, C₁₋₃-fluoroalkyl, —C₁₋₃-alkylene-OR^{2.1}, —C₁₋₃-alkylene-NR^{2.2}R^{2.3}, —NR^{2.2}R^{2.3}, O—R^{2.1}; SO—R^{2.1}, SO₂—R^{2.1}, COOR^{2.1}, —CO—NH—(C₁₋₆-alkylene)-hetaryl, —CO—NH-hetaryl, —CO—N(CH₃)-het, —CO—N(CH₃)—(C₁₋₃-alkylene)-het, —CO—N(CH₃)—(C₁₋₃-alkylene)-hetaryl, —CO—N(C₃₋₇-cycloalkyl)-het, —CO—NR^{2.2}R^{2.3}, —CO—NH—(C₁₋₆-alkylene)-het, NR^{2.2}—CO—R^{2.1}, C₆₋₁₀-aryl, C₆₋₁₀-aryl-C₁₋₂-alkylene, het-C₁₋₂-alkylene, -het, —CO-het, CO—N(CH₃)—C₃₋₇-cycloalkyl, C₃₋₇-cycloalkyl, C₃₋₇-cycloalkyl-C₁₋₂-alkylene, hetaryl-C₁₋₂-alkylene and hetaryl,
   wherein this group may optionally be substituted by one or more groups selected from among OH, halogen, —C₁₋₃-fluoroalkyl, oxo, methyl and phenyl,
      or wherein
R³ is a group selected from among het and hetaryl, which may optionally be substituted by one or more groups selected from among halogen, C₁₋₃-fluoroalkyl, CN, OH, oxo, —C₁₋₆-alkyl, —C₁₋₃-alkylene-NR^{2.2}R^{2.3}, —NR^{2.2}R^{2.3}, SO—R^{2.1}, SO₂—R^{2.1}, —O—R^{2.1}, —COOR^{2.1}, SO₂—(CH₃), SO₂—(CH₂—CH₃), C₆₋₁₀-aryl, het, C₃₋₇-cycloalkyl and hetaryl,
   which in turn may optionally be substituted by one or more groups selected from among OH, halogen, —C₁₋₃-fluoroalkyl, C₁₋₆-alkyl, C₆₋₁₀-aryl, —COO(C₁₋₃-alkyl) and O—(C₁₋₃-alkyl) may be substitued,
      or wherein
R³ denotes —O—R^{3.1},
wherein R^{3.1} is a group selected from among —C₁₋₆-alkyl, —C₆₋₁₀-aryl, —C₁₋₃-alkylene-C₆₋₁₀-aryl, hetaryl and het,
   which may optionally be substituted in the ortho, para or meta position by one, two or three groups independently of one another selected from among fluorine, chlorine, bromine, hydroxy, CN, C₁₋₆-alkyl, C₁₋₃-fluoroalkyl, CO—(C₁₋₅-alkyl), —CO—(C₁₋₃-fluoroalkyl), —CO—NH—(C₁₋₆-alkylene)-hetaryl, —CO—N(C₁₋₃-alkyl)-(C₁₋₆-alkylene)-hetaryl,
   —CO—N(C₁₋₃-alkyl)-het, —CO—N(C₃₋₇-cycloalkyl)-het, —C₁₋₃-alkylene-OR^{2.1}, —C₁₋₃-alkylene-NR^{2.2}R^{2.3}, —NR^{2.2}R^{2.3}, O—R^{2.1}SO—R^{2.1}SO₂—R^{2.1}, COOH, COO—(C₁₋₄-alkyl), —O—C₁₋₃-alkylene-N(C₁₋₃-alkyl)₂, CO—NR^{2.2}R^{2.3}, NR^{2.2}—CO—R^{2.1}, C₆₋₁₀-aryl, C₆₋₁₀-aryl-C₁₋₂-alkylene, het-C₁₋₂-alkylene, —CO-het, het, —CO—C₃₋₇-cycloalkyl, —CO—N(C₁₋₃-alkyl)-C₃₋₇-cycloalkyl C₃₋₇-cycloalkyl, C₃₋₇-cycloalkyl-C₁₋₂-alkylene, hetaryl-C₁₋₂-alkylene and hetaryl,
   which in turn may optionally be substituted by 1, 2, 3 or 4 groups selected independently of one another from among F, Cl, Br, methyl, O— methyl, ethyl, O-ethyl, OH, oxo and CF₃.
      and wherein
R⁴ denotes H, CN, OH, CF₃, CHF₂, CH₂F, F, methyl, ethyl, —O—(C₁₋₃-alkyl), —C₁₋₃-alkylene-OH, —COO(C₁₋₃-alkyl), —CO-het, —(C₁₋₂-alkylene)-NH—SO₂—(C₁₋₂-alkyl), —(C₁₋₂-alkylene)-N(C₁₋₃-alkyl)-SO₂—(C₁₋₂-alkyl), —(C₁₋₂-alkylene)-O—(C₁₋₂-alkylene)-C₆₋₁₀-aryl, —C₁₋₃-alkylene-O—C₁₋₃-alkyl, —(C₁₋₂-alkylene)-N(C₁₋₃-alkyl)-CO—(C₁₋₂-alkyl), —NH—CO—(C₁₋₃-alkylene)-O—(C₁₋₃-alkyl), —C₁₋₃-alkylene-NH—CO—(C₁₋₃-alkyl), —C₁₋₃-alkylene-NH—CO—(C₁₋₃-alkylene)-N(C₁₋₃-alkyl)₂, —O—(C₁₋₂-alkylene)-(C₆₋₁₀-aryl), —C₁₋₃-alkylene-NH—CO—(C₁₋₃-alkylene)-O—(C₁₋₃-alkyl), —CO—(C₆₋₁₀-aryl), —(C₁₋₂-alkylene)-N(C₁₋₃-alkyl)-CO—(C₁₋₂-alkylene)-O—(C₁₋₃-alkyl),
   wherein the aryl in the above groups may optionally be substituted by one or more groups selected from among F, Cl, Br, methyl, ethyl, propyl, isopropyl, cyclopropyl, —O-methyl, —O-ethyl, —O-propyl, —O-isopropyl, —O-cyclopropyl, —OH and CF₃,
      or wherein
R³ and R⁴ together form a mono- or bicyclic, unsaturated, saturated or partly saturated heterocyclic group, which contains 1, 2 or 3 heteroatoms selected from among N, O and S and which may optionally be substituted by one or more groups selected from among halogen, OH, oxo, C₁₋₃-fluoroalkyl, CN, C₁₋₆-alkyl, —O—R^{2.1}, —COOR^{2.1}, SO—R^{2.1}, SO₂—R^{2.1}, —C₁₋₃-alkylene-NR^{2.2}R^{2.3}, —NR^{2.2}R^{2.3}, C₆₋₁₀-aryl, C₃₋₇-cycloalkyl, het and hetaryl.

Also preferred are the above mentioned medicament combinations, which comprises at least one EP4 receptor antagonist (2), in addition to one or more, preferably one, PDE4 inhibitor (1) of general formula 1,

wherein
X is SO,
R¹ is H
R² is H or C₁₋₆-alkyl, which may optionally be substituted by one or more groups selected from F, Cl, CF₃, CHF₂ or CH₂F or which may optionally be substituted by one or more groups selected from among OR^{2.1}, COOR^{2.1}, CONR^{2.2}R^{2.3}, SR^{2.1}, SO—R^{2.1}, SO₂—R^{2.1}, phenyl, het, hetaryl, a monocyclic C₃₋₇-cycloalkyl, CH₂—NR^{2.2}R^{2.3} and NR^{2.2}R^{2.3}, which in turn may optionally be substituted by one or more groups selected from among OH, F, Cl, Br, CF₃, CHF₂, CH₂F, OR^{2.1}, oxo, methyl, ethyl, propyl, isopropyl, methanol, ethanol, phenyl, COOR^{2.1}, CH₂—NR^{2.2}R^{2.3} and NR^{2.2}R^{2.3},
   wherein
het is a three- to seven-membered, monocyclic, saturated or partly saturated heterocyclic group or a seven- to eleven-membered, bicyclic, saturated or partly saturated heterocyclic group which contains 1, 2 or 3 heteroatoms selected independently of one another from among N, S or O,
   and wherein
hetaryl is a five- to six-membered, monocyclic, aromatic heteroaryl, or a seven- to eleven-membered, bicyclic, aromatic heteroaryl which contains 1, 2 or 3 heteroatoms selected independently of one another from among N, S or O,
   and wherein
cycloalkyl may be saturated or partly saturated,
   wherein R^{2.1} is H or a group selected from among methyl, ethyl, propyl, isopropyl, methanol, ethanol, monocyclic C₃₋₇ cycloalkyl, phenyl-C₁₋₂-alkylene, -hetaryl-C₁₋₂-alkylene, -het-C₁₋₂-alkylene, C₃₋₇-cycloalkyl-C₁₋₂-alkylene, phenyl, hetaryl and a het,
   which may optionally be substituted by one or more groups selected from among OH, F, Cl, methyl, ethyl, propyl, isopropyl, O-methyl, O-ethyl, O-propyl, O-isopropyl and phenyl,
   wherein R^{2.2} and R^{2.3} independently of one another denote H or a group selected from among methyl, ethyl, propyl, isopropyl, monocyclic C₃₋₇cycloalkyl, phenyl-C₁₋₃-alkylene, hetaryl-C₁₋₃-alkylene, phenyl, -het, -hetaryl, CO—NH₂, CO—NHCH₃, CON(CH₃)₂, SO₂—(C₁₋₂-alkyl), CO—R^{2.1} and COOR^{2.1},
   which may optionally be substituted by one or more groups selected from among OH, F, Cl, methyl, ethyl, propyl, isopropyl, phenyl and COOR^{2.1},
      or
R² denotes a monocyclic C₃₋₇ cycloalkyl, which may optionally be substituted by a group selected from among C₁₋₂-alkanol, C₁₋₃-fluoroalkyl, C₁₋₃-alkylene-OR^{2.1}, OR^{2.1}, COOR^{2.1}, SO₂—NR^{2.2}R^{2.3}, -het, —NH—CO—O-(phenyl), methyl, ethyl, propyl, isopropyl, phenyl, phenyl-C₁₋₂-alkylene, -hetaryl-C₁₋₂-alkylene, monocyclic C₃₋₇ cycloalkyl and NR^{2.2}R^{2.3},
   which may optionally be substituted by one or more groups selected from among OH, OR^{2.1}, oxo, F, Cl, CF₃, CHF₂, CH₂F, methyl, ethyl, propyl, isopropyl, phenyl and NR^{2.2}R^{2.3},
      or
R² denotes a phenyl which may optionally be substituted by OH, SH, F, Cl or Br or by one or more groups selected from among OR^{2.1}, COOR^{2.1}, NR^{2.2}R^{2.3}, CH₂—NR^{2.2}R^{2.3}, monocyclic C₃₋₇-cycloalkyl, -het, methyl, ethyl, propyl, isopropyl, CF₃, CHF₂, CH₂F, phenyl-C₁₋₂-alkylene, het-C₁₋₂-alkylene, hetaryl-C₁₋₂-alkylene, phenyl, SO₂—CH₃, SO₂—CH₂CH₃ and SO₂—NR^{2.2}R^{2.3},
   which in turn may optionally be substituted by one or more or several groups selected from among OH, OR^{2.1}, oxo, F, Cl, CF₃, CHF₂, CH₂F, methyl, ethyl, propyl, isopropyl, phenyl and NR^{2.2}R^{2.3},
      or
R² denotes a group selected from among het and hetaryl,
   which may optionally be substituted by one or more groups selected from among F, Cl, OH, oxo, CF₃, CHF₂ and CH₂F or by one or more groups selected from among OR^{2.1}, C₁₋₃-alkylene-OR^{2.1}, SR^{2.1}, SO—R^{2.1}, SO₂—R^{2.1}, COOR^{2.1}, COR^{2.1}, methanol, ethanol, monocyclic C₃₋₇-cycloalkyl, phenyl, methyl, ethyl, propyl, isopropyl, phenyl-C₁₋₂-alkylene, hetaryl-C₁₋₂-alkylene, -het, -hetaryl and NR^{2.2}R^{2.3},
   which may in turn optionally be substituted by one or more groups selected from among OH, OR^{2.1}, oxo, F, Cl, CF₃, CHF₂, CH₂F, methyl, ethyl, propyl, isopropyl, butyl, isobutyl, phenyl and NR^{2.2}R^{2.3},
      and wherein
R³ is a naphthalene or phenyl,
   which may optionally be substituted in the ortho, para or meta position by one or two groups independently of one another selected from among fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO—CH₃, COOCH₃, COOCH₂CH₃, —CO—NH-(methylene)-hetaryl, —CO—NH-(ethylene)-hetaryl, —CO—NH-hetaryl, —CO—N(CH₃)-het, —CO—N(CH₃)-(methylene)-het, —CO—N(CH₃)-(ethylene)-het, —CO—N(CH₃)-(methylene)-hetaryl, —CO—N(CH₃)-(ethylene)-hetaryl, —CO—N(cyclopropyl)-het, CO—NH₂, CONH(CH₃), CON(CH₃)₂, —CO—NH-(methylene)-het, —CO—NH-(ethylene)-het, —NH—CO-methyl, NCH₃—CO-methyl, —NH—CO-ethyl, NCH₃—CO-ethyl, —NH—CO-propyl,
   NCH₃—CO-propyl, —NH—CO-isopropyl, NCH₃—CO-isopropyl, phenyl, phenyl-methylene, phenyl-ethylene, het-methylene, het-ethylene, -het, —CO-het, —CO—N(CH₃)-het, CO—N(CH₃)-cyclopropyl, C₃₋₇-cycloalkyl, C₃₋₇-cycloalkyl-methylene, C₃₋₇-cycloalkyl-ethylene, hetaryl-methylene, hetaryl-ethylene, -hetaryl, CH₂—NH₂, CH₂—NH(CH₃), CH₂—N(CH₃)₂, —NH₂, —NH(CH₃) and —N(CH₃)₂,
   wherein this group may optionally be substituted by one or more groups selected from among OH, F, Cl, —CF₃, CHF₂, CH₂F, oxo, methyl and phenyl
      or wherein
R³ denotes a group selected from among a het and hetaryl which may optionally be substituted by one or more groups selected from among F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO—(CH₃), SO—(CH₂—CH₃), SO₂—(CH₃), SO₂—(CH₂—CH₃), phenyl, CH₂—NH₂, CH₂—NH(CH₃), CH₂—N(CH₃)₂, —NH₂, —NH(CH₃), —N(CH₃)₂, het and hetaryl,
   which in turn may optionally be substituted by one or more groups selected from among OH, F, Cl, CF₃, CHF₂, CH₂F, methyl, ethyl, propyl, isopropyl, phenyl, —COO-methyl, —COO-ethyl and O-methyl, O-ethyl,
      or wherein
R³ denotes —O—R^{3.1},
   wherein
R³¹ is a group selected from among —C₁₋₃-alkyl, -phenyl, —C₁₋₃-alkylene-phenyl, hetaryl and het,
   which may optionally be substituted in the ortho, para or meta position by one, two or three groups independently of one another selected from among fluorine, chlorine, bromine, hydroxy, CN, methyl, ethyl, propyl, isopropyl, butyl, isobutyl, CF₃, CHF₂, CH₂F, CO-(methyl), CO-(ethyl),
   CO-(propyl), CO-(isopropyl), —CO—(CF₃), —CO—NH-(methylene)-hetaryl, —CO—NH-(ethylene)-hetaryl,
   CO—N(CH₃)-(methylene)-hetaryl, —CO—N(CH₃)-(ethylene)-hetaryl, —CO—N(CH₃)-(propylene)-hetaryl, —CO—N(CH₃)-(isopropylene)-hetaryl —CO—N(CH₃)-het, —CO—N(cyclopropyl)-het, —CO—N(C₅₋₇-cycloalkyl)-het, -methylene-O-methyl, -ethylene-O-methyl, -propylene-O-methyl, -methylene-O-ethyl, -ethylene-O-ethyl, -propylene-O-ethyl, -methylene-NH₂, -methylene-NHCH₃, -methylene-N(CH₃)₂, -ethylene-NH₂,
   -ethylene-NHCH₃, -ethylene-N(CH₃)₂, NH₂, N(CH₃)₂, NHCH₃, —O-methyl, O-ethyl, O-propyl, O-isopropyl, O-butyl, O-isobutyl, —SO—CH₃, SO-ethyl, —SO-propyl, —SO-isopropyl, SO₂-methyl, —SO₂-ethyl, SO₂-propyl, SO₂-isopropyl, COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NH(CH₃), CO—N(CH₃)₂, —NH—CO-methyl, —NCH₃—CO-methyl, —NH—CO-ethyl, NCH₃—CO-ethyl, phenyl, phenyl-methylene, phenyl-ethylene, het-methylene, het-ethylene, —CO-het, het,
   —CO—C₅₋₇-cycloalkyl, —CO-cyclopropyl, —CO—N(CH₃)—C₅₋₇-cycloalkyl, —CO—N(CH₃)-cyclopropyl,
   C₅₋₇-cycloalkyl, cyclopropyl, C₅₋₇-cycloalkyl-methylene, C₅₋₇-cycloalkyl-ethylene, cyclopropyl-methylene, cyclopropyl-ethylene, hetaryl-methylene, hetaryl-ethylene and hetaryl,
   which in turn may optionally be substituted by 1, 2, 3 or 4 groups selected independently of one another from among F, Cl, Br, methyl, O-methyl, ethyl, O-ethyl, OH, oxo and CF₃,
      and wherein
R⁴ denotes H, CN, OH, CF₃, CHF₂, CH₂F, F, methyl, ethyl, O-methyl or O-ethyl, -methylene-OH, -ethylene-OH, -propylene-OH, isopropylene-OH,
   —COO(methyl), —COO(ethyl), —COO(propyl), —COO(isopropyl), —CO-het, -(methylene)-NH—SO₂-(methyl), -(methylene)-NH—SO₂-(ethyl),
   -(ethylene)-NH—SO₂-(methyl), -(ethylene)-NH—SO₂-(ethyl),
   -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl
   -ethylene-O-ethyl,
   -(methylene)-N(CH₃)—CO-(methyl), -(methylene)-N(CH₃)—CO-(ethyl) -(ethylene)-N(CH₃)—CO-(methyl), -(ethylene)-N(CH₃)—CO-(ethyl), —NH—CO-(methylene)-O-(methyl), —NH—CO-(methylene)-O-(ethyl), —NH—CO-(ethylene)-O-(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene)-N(CH₃)—CO-(ethylene)-O-(ethyl), -(ethylene)-N(CH₃)—CO-(methylene)-O-(ethyl), —O-(methylene)-phenyl, —O-(ethylene)-phenyl,
   —CO-phenyl,
   wherein the phenyl in the above groups may optionally be substituted by one or more other groups selected from among F, Cl, Br, methyl, ethyl, propyl, —O-methyl, —O-ethyl, —O-propyl, —OH and CF₃
      or wherein
R³ and R⁴ together form a mono- or bicyclic, unsaturated, saturated or partly saturated heterocyclic group, which contains 1, 2 or 3 heteroatoms selected from among N, O and S and which may optionally be substituted by one or more groups selected from among F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂CH₃), SO—(CH₃), SO—(CH₂CH₃), CH₂—NH₂, CH₂—NH(CH₃), CH₂—N(CH₃)₂, —NH₂, —NH(CH₃), —N(CH₃)₂, phenyl, C₅₋₇-cycloalkyl, het and hetaryl,

In another particularly preferred aspect the invention relates to those of the above mentioned medicament combinations which contain at least one EP4 receptor antagonist (2), in addition to one or more, preferably one PDE4 inhibitor of general formula 1, wherein
R² denotes a group of formula 3

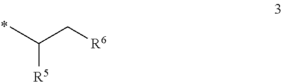

wherein R⁶ is OH or NH₂ and
wherein R⁵ denotes a group selected from among C₁₋₄-alkyl, a five- to six-membered heteroaryl with 1, 2 or 3 heteroatoms selected from among S, O and N and phenyl, which may optionally be substituted by one or more groups selected from among OH, F, Br, OR^{2.1}, oxo, methyl, ethyl, methanol, ethanol, phenyl, COOR^{2.1}, CH₂—NR^{2.2}R^{2.3} and NR^{2.2}R^{2.3}.

In another particularly preferred embodiment the present invention relates to those of the above mentioned medicament combinations which comprise at least one EP4 receptor antagonist (2), in addition to one or more, preferably one, PDE4 inhibitor of general formula 1, wherein
R² is a group according to formula 3

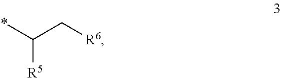

wherein R⁶ is OH or NH₂ and
wherein R⁵ denotes methyl, ethyl, propyl or isopropyl,

The present invention also preferably relates to those of the above medicament combinations which comprises at least one EP4 receptor antagonist (2) in addition to one or more, preferably one, PDE4 inhibitor of general formula 1, wherein
R² is a monocyclic three, four, five, six or seven-membered cycloalkyl ring which may optionally be substituted in the spiro position by a group selected from among —CH₂—OR^{2.1}, branched or unbranched C₂₋₆-alkylene-OR^{2.1}, methyl, ethyl, propyl, isopropyl, butyl, isobutyl, cyclopropyl, —CF₃, CHF₂, CH₂F and C₂₋₄-fluoroalkyl, and wherein R^{2.1} is selected from among methyl, ethyl, propyl, isopropyl, butyl, isobutyl,

In another particularly preferred embodiment the present invention relates to those of the above mentioned medicament combinations which comprises at least one EP4 receptor antagonist (2) in addition to one or more, preferably one, PDE4 inhibitor of general formula 1, wherein
R² is a cyclopropyl which may optionally be substituted by another group selected from among —NH₂, CH2—NH₂, —NH(CH₃), —N(CH₃)₂, methyl, ethyl, propyl, isopropyl, —NH—CO-(tert-butyl), —NH—CO—O-(tert-butyl), —N(CH₃)—CO-(tert-butyl), —N(CH₃)—CO—O-(tert-butyl), —CF₃, —CHF₂, CH₂F, F, Cl and Br,

In another preferred aspect the present invention relates to those of the above mentioned medicament combinations which comprises at least one EP4 receptor antagonist (2) in addition to one or more, preferably one PDE4 inhibitor of general formula 1, wherein
R² is a phenyl which may optionally be substituted in one or both meta positions by one or more groups selected from among methyl, ethyl, propyl, isopropyl, cyclopropyl, F, Cl, Br, OH, OR^{2.1}, COOR^{2.1}, CF₃, CHF₂, CH₂F, NH₂, NH(CH₃) and N(CH₃)₂, wherein R^{2.1} may be H, methyl or ethyl,

Within the scope of the present invention one of the above medicament combinations is also particularly preferred, which contains at least one EP4 receptor antagonist (2) in addition to one or more, preferably one PDE4 inhibitor of general formula 1, wherein
R² is a group selected from among a monocyclic, saturated three, four, five, six or seven-membered heterocyclic group with 1, 2 or 3 heteroatoms selected in each case from among N, O and S, which may optionally be substituted by one or more groups selected from among fluorine, chlorine, bromine, CF₃, CHF₂, CH₂F, OH and oxo or by one or more groups selected from among OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, C₃₋₁₀-cycloalkyl, phenyl, C₁₋₆-alkyl, phenyl-C₁₋₆-alkylene, hetaryl-C₁₋₆-alkylene, het, hetaryl and NR^{2.2}R^{2.3}, which may in turn optionally be substituted by one or more groups selected from among OH, OR^{2.1}, oxo, F, Cl, CF₃, CHF₂, CH₂F, C₁₋₆-alkyl, phenyl and NR^{2.2}R^{2.3},
   wherein R^{2.1}, R^{2.2} and R^{2.3} are defined as in claim 1.

In another particularly preferred aspect the invention relates to a medicament combination which contains at least one EP4 receptor antagonist (2) in addition to one or more, preferably one, PDE4 inhibitor of general formula 1, wherein
R² denotes a group selected from among a monocyclic, saturated six-membered heterocyclic group with a heteroatom selected from among N, O and S, which may optionally be substituted by one or more groups selected from among F, Cl, Br, CF₃, CHF₂, CH₂F, OH, oxo, NH₂, NHCH₃, N(CH₃)₂, methyl, ethyl, propyl, isopropyl, cyclopropyl, methoxy and ethoxy.

Also particularly preferred is one of the above medicament combinations which comprises at least one EP4 receptor antagonist (2) in addition to one or more, preferably one, PDE4 inhibitor of general formula 1, wherein
R² denotes a group selected from among piperidine or tetrahydropyran, which may optionally be substituted by one or more groups selected from among F, Cl, Br, OH, CF₃, CHF₂, CH₂F, NH₂, NHCH₃, N(CH₃)₂, oxo, methyl and methoxy.

In another particularly preferred aspect the invention relates to one of the above mentioned medicament combinations, which comprises at least one EP4 receptor antagonist (2) in addition to one or more, preferably one, PDE4 inhibitor of general formula 1, wherein
R³ is a naphthalene or phenyl,
   which may optionally be substituted in any desired position by one, two or three groups selected independently of one another from among fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO₂—CH₂CH₃, COOCH₃ and CO—O—CH₂CH₃.

Also particularly preferred is one of the above medicament combinations, which comprises at least one EP4 receptor antagonist (2) in addition to one or more, preferably one, PDE4 inhibitor of general formula 1, wherein
R³ is a group selected from among het and hetaryl, which may optionally be substituted by one or more groups selected from among F, Cl, Br, CF₃, CHF₂, CH₂F, CN, OH, oxo, methyl, ethyl, propyl, isopropyl, butyl, isobutyl, cyclopropyl, C₅₋₇-cycloalkyl, —O-methyl, —O-ethyl, —O-propyl, —O-isopropyl, —COO-methyl, —COO-ethyl, —COO-propyl, —COO-isopropyl, SO₂—(CH₃), SO₂—(CH₂—CH₃), SO—(CH₃), SO—(CH₂—CH₃), phenyl, —CH₂—NH₂, —CH₂—NHCH₃, —CH₂—N(CH₃)₂, NH₂, NHCH₃, N(CH₃)₂, het and hetaryl, which may in turn optionally be substituted by one or more groups selected from among OH, F, Cl, Br, CF₃, CHF₂, CH₂F, methyl, ethyl, propyl, isopropyl, phenyl, —COO-methyl, —COO-ethyl, —COO-propyl, —COO-isopropyl and O-methyl, O-ethyl, O-propyl and O-isopropyl,
   and wherein
R⁴ denotes H, CN, OH, CF₃, CHF₂, CH₂F, F, methyl, ethyl, O-methyl or O-ethyl, and
   wherein
het is a three- to seven-membered, monocyclic, saturated or partly saturated heterocyclic group or ein seven- to eleven-membered, bicyclic, annellated, saturated or partly saturated heterocyclic group, which contains 1, 2 or 3 heteroatoms selected independently of one another from among N, S or O,
   and wherein
hetaryl is a five- to six-membered, monocyclic, aromatic heteroaryl or a seven- to eleven-membered, bicyclic, annellated, aromatic heteroaryl which contains in each case 1, 2 or 3 heteroatoms selected independently of one another from among N, S or O,
   and wherein
cycloalkyl may be saturated or partly saturated,

In another particularly preferred aspect the invention relates to one of the above mentioned medicament combinations, which comprises at least one EP4 receptor antagonist (2) in addition to one or more, preferably one, PDE4 inhibitor of general formula 1, wherein
R³ denotes a group selected from a bicyclic, seven- to eleven-membered, saturated or partly saturated heterocyclic group or a bicyclic, seven- to eleven-membered heteroaryl, which is selected from among indole, dihydroindole, quinazoline, dihydroquinazoline, tetrahydroquinazoline, benzoisoxazole, dihydrobenzoisoxazole, benzoxazine, dihydrobenzoxazine, benzothiazole, dihydrobenzothiazole, triazolopyridine, dihydrotriazolopyridine, benzofuran, dihydrobenzofuran, isobenzofuran and dihydroisobenzofuran,
   which may optionally be substituted by one or more groups selected from among F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO₂—(CH₃), SO₂—(CH₂—CH₃), SO—(CH₃), SO—(CH₂—CH₃), phenyl, —CH₂—NH₂, —CH₂—NHCH₃, —CH₂—N(CH₃)₂, NH₂, NHCH₃, N(CH₃)₂, furanyl and pyridinyl,
   which may in turn be substituted by one or more groups selected from among OH, F, Cl, Br, CF₃, CHF₂, CH₂F, methyl, ethyl, propyl, isopropyl, phenyl, —COO-methyl, —COO-ethyl and O-methyl, O-ethyl,

In another particularly preferred aspect the invention relates to one of the above mentioned medicament combinations, which comprises at least one EP4 receptor antagonist (2) in addition to one or more, preferably one, PDE4 inhibitor of general formula 1, wherein
R³ is a group selected from a monocyclic, saturated or partly saturated, three- to seven-membered heterocyclic group or a monocyclic five- to six-membered heteroaryl, which is selected from among imidazole, dihydroimidazole, oxadiazole, oxadiazolidine, pyrazole, pyridine and dihydropyrazole,
   which may optionally be substituted by one or more groups selected from among F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO₂—(CH₃), SO₂—(CH₂—CH₃), SO—(CH₃), SO—(CH₂—CH₃), phenyl, —CH₂—NH₂, —CH₂—NHCH₃, —CH₂—N(CH₃)₂, NH₂, NHCH₃, N(CH₃)₂, furanyl and pyridinyl, which may in turn be substituted by one or more groups selected from among OH, F, Cl, Br, CF₃, CHF₂, CH₂F, methyl, ethyl, propyl, isopropyl, phenyl, —COO-methyl, —COO-ethyl and O-methyl, O-ethyl,

Also particularly preferred is one of the above medicament combinations, which comprises at least one EP4 receptor antagonist (2) in addition to one or more, preferably one, PDE4 inhibitor of general formula 1, wherein
R³ and R⁴ together form a mono- or bicyclic, unsaturated or partly saturated, three- to eleven-membered heterocyclic group which contains 1, 2 or 3 heteroatoms selected from among N, O and S and which may optionally be substituted by one or more groups selected from among F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂—CH₃), SO—(CH₃), SO—(CH₂—CH₃), phenyl, —CH₂—NH₂, —CH₂NHCH₃, —CH₂—N(CH₃)₂, NH₂, NHCH₃, N(CH₃)₂, a saturated or partly saturated, five- to six-membered heterocyclic group and a five- to six-membered heteroaryl,

In another particularly preferred aspect the invention relates to one of the above mentioned medicament combinations, which comprises at least one EP4 receptor antagonist (2) in addition to one or more, preferably one, PDE4 inhibitor of general formula 1, wherein
R³ and R⁴ together form a bicyclic heterocyclic group selected from among tetrahydroquinazoline, tetrahydrobenzoxazin and dihydroindole, dihydroisobenzofuran which may optionally be substituted by one or more groups selected from among F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂—CH₃), phenyl, —CH₂—NH₂, —CH₂NHCH₃, —CH₂—N(CH₃)₂, NH₂, NHCH₃, N(CH₃)₂, a saturated or partly saturated, five- or six-membered heterocyclic group and a five- or six-membered heteroaryl,

Also particularly preferred is one of the above medicament combinations, which comprises at least one EP4 receptor antagonist (2) in addition to one or more, preferably one, PDE4 inhibitor of general formula 1, wherein
R³ is —O—R³¹,
R^{3.1} is a group selected from among methyl, ethyl, propyl, isopropyl, butyl, isobutyl, pentyl, isopentyl, -phenyl, -methylene-phenyl, -ethylene-phenyl, -propylene-phenyl, -isopropylene-phenyl, hetaryl and het,
   which may optionally be substituted in the ortho, para or meta position by one, two or three groups selected independently of one another from among fluorine, chlorine, bromine, hydroxy, CN, methyl, ethyl, propyl, isopropyl, butyl, isobutyl, —CF₃, CHF₂, CH₂F, CO-(methyl), CO-(ethyl), CO-(propyl), CO-(isopropyl), CO-(butyl), CO-(isobutyl), —CO—(CF₃), —CO—(CH₂F), —CO—(CHF₂), —CO—NH-(methylene)-hetaryl, —CO—NH-(ethylene)-hetaryl, —CO—NH-(propylene)-hetaryl, —CO—NH-(isopropylene)-hetaryl, —CO—N(CH₃)-(methylene)-hetaryl, —CO—N(CH₃)-(ethylene)-hetaryl, —CO—N(CH₃)-(propylene)-hetaryl, —CO—N(CH₃)-(isopropylene)-hetaryl, —CO—N(CH₃)-het, —CO—N(C₃₋₇-cycloalkyl)-het, -methylene-O-methyl, -ethylene-O-methyl, -methylene-O-ethyl, -ethylene-O-ethyl, -methylene-NH₂, -ethylene-NH₂, -methylene-NHCH₃, -ethylene-NHCH₃, -methylene-N(CH₃)₂, -ethylene-N(CH₃)₂, —NH₂, —NHCH₃, —N(CH₃)₂, —O-methyl, —O-ethyl, —O-propyl, —O-isopropyl, —SO—CH₃, —SO—(CH₂CH₃), —SO₂—CH₃, —SO₂—(CH₂CH₃), COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NHCH₃, CO—N(CH₃)₂, NH—CO-methyl, NCH₃—CO-methyl, NH—CO-ethyl, N(CH₃)—CO-ethyl, phenyl, phenyl-methylene, phenyl-ethylene, het-methylene, het-ethylene, —CO-het, het, —CO—C₄₋₇-cycloalkyl, —CO-cyclopropyl, —CO—N(CH₃)-cyclopropyl, —CO—N(CH₃)—C₄₋₇-cycloalkyl, C₄₋₇-cycloalkyl, cyclopropyl, C₄₋₇-cycloalkyl-methylene, cyclopropyl-methylene, C₄₋₇-cycloalkyl-ethylene, cyclopropyl-ethylene, hetaryl-methylene, hetaryl-ethylene- and hetaryl,
   which in turn may optionally be substituted by 1, 2, 3 or 4 groups selected independently of one another from among F, Cl, Br, methyl, O-methyl, ethyl, O-ethyl, OH, oxo and CF₃,

In another particularly preferred aspect the invention relates to one of the above mentioned medicament combinations, which comprises at least one EP4 receptor antagonist (2) in addition to one or more, preferably one, PDE4 inhibitor of general formula 1, wherein
R⁴ is selected from among
   H, CN, OH, CF₃, CHF₂, CH₂F, F, methyl, ethyl, O-methyl, O-ethyl, -methylene-OH, -ethylene-OH, -propylene-OH, isopropylene-OH,
   —COO(methyl), —COO(ethyl), —COO(propyl), —COO(isopropyl), —CO-het, -(methylene)-NH—SO₂-(methyl), -(methylene)-NH—SO₂-(ethyl), -(ethylene)-NH—SO₂-(methyl), -(ethylene)-NH—SO₂-(ethyl), -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl
   -ethylene-O-ethyl,
   -(methylene)-N(CH₃)—CO-(methyl), -(methylene)-N(CH₃)—CO-(ethyl) -(ethylene)-N(CH₃)—CO-(methyl), -(ethylene)-N(CH₃)—CO-(ethyl), —NH—CO-(methylene)-O-(methyl), —NH—CO-(methylene)-O-(ethyl), —NH—CO-(ethylene)-O-(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene)-N(CH₃)—CO-(ethylene)-O-(ethyl), -(ethylene)-N(CH₃)—CO-(methylene)-O-(ethyl), —O-(methylene)-phenyl, —O-(ethylene)-phenyl,
   —CO-phenyl,
   and wherein the phenyl in the above groups may optionally be substituted by one or more other groups selected from among F, Cl, Br, methyl, ethyl, propyl, —O-methyl, —O-ethyl, —O-propyl, —OH and CF₃,

In another particularly preferred aspect the invention relates to one of the above mentioned medicament combinations, which comprises at least one EP4 receptor antagonist (2) in addition to one or more, preferably one, PDE4 inhibitor of general formula 1, wherein
R³ is a group selected from among oxazole, imidazol and thiazole, wherein this group may optionally be substituted by one, two or three further groups selected independently of one another from among methyl, ethyl, propyl, isopropyl, O-methyl, O-ethyl, O-propyl, O-isopropyl, OH, F, Cl, Br, CF₃, phenyl, hetaryl and C₃₋₆-cycloalkyl,

In another particularly preferred aspect the invention relates to one of the above mentioned medicament combinations, which comprises at least one EP4 receptor antagonist (2) in addition to one or more, preferably one, PDE4 inhibitor of general formula 1, wherein
X is SO₂,

In another particularly preferred aspect the invention relates to one of the above mentioned medicament combinations, which comprises at least one EP4 receptor antagonist (2) in addition to one or more, preferably one, PDE4 inhibitor of general formula 1 selected from among:
1.1. (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol
1.2. (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol
1.3. (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol
1.4. (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol
1.5. (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one
1.6. {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine
1.7. 1-(4-(1-hydroxymethylcyclopropylamino)-5-oxo-6,7-dihydro-5H-5λ⁴-thieno[3,2-d]pyrimidin-2-yl)-3′-methyl-1′H-spiro[piperidin-4,4′-quinazolin]-2′(3′H)-one
1.8. {1-[2-(4-benzo[c]isoxazol-3-yl-piperidin-1-yl)-5-oxo-6,7-dihydro-5H-5λ⁴-thieno[3,2d]pyrimidin-4-ylamino]-cyclopropyl}-methanol
1.9. (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol
1.10. 1-[4-((S)-1-methyl-6-oxopiperidin-3-ylamino)-5-oxo-6,7-dihydro-5H-5λ⁴-thieno[3,2-d]pyrimidin-2-yl]-4-phenylpiperidin-4-carbonitrile
1.11. 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(3′H)-one
1.12. (3-fluorophenyl)-[5-oxo-2-(3,4,5,6-tetrahydro-2H[4,4′]bipyridinyl-1-yl)-6,7-dihydro-5H-5λ⁴-thieno[3,2-d]pyrimidin-4-yl]amine
1.13. {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine
1.14. (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol
1.15. {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine
1.16. (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one
1.17. (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.18. (1-{2-[4-(5-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.19. {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.20. (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine
1.21. (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol
1.22. (S)-5-{2-[4-(4-fluorophenoxy)-piperidin-1-yl]-5-oxo-6,7-dihydro-5H-5λ⁴-thieno[3,2-d]pyrimidin-4-ylamino}-1-methylpiperidin-2-one
1.23. (2-{4-[4-(4,5-dihydroxazol-2-yl)-phenoxy]-piperidin-1-yl}-5-oxo-6,7-dihydro-5H-5λ⁴-thieno[3,2-d]pyrimidin-4-yl)-(tetrahydropyran-4-yl)-amine
1.24. 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid
1.25. 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol
1.26. {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine
1.27. 2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl)-(tetrahydropyran-4-yl)-amine
1.28. (S)-5-(2-{4-[4-(4,5-dihydroxazol-2-yl)-phenoxyl]-piperidin-1-yl}-5-oxo-6,7-dihydro-5H-5λ⁴-thieno[3,2-d]pyrimidin-4-ylamino)-1-methylpiperidin-2-one
1.29. {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.30. {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine
1.31. 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide
1.32. N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide
1.33. N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yloxy}-benzamide
1.34. (5-oxo-2-[4-(pyridin-4-yloxy)-piperidin-1-yl]-6,7-dihydro-5H-5λ⁴-thieno[3,2-d]pyrimidin-4-yl)-(tetrahydropyran-4-yl)-amine
1.35. {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine
1.36. (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one
1.37. (1-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-cyclopropyl)-methanol
1.38. (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one
1.39. {4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-yl}-methanol
1.40. [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol
1.41. 4-(4-chlorophenyl)-1-[5-oxo-4-(tetrahydropyran-4-ylamino)-6,7-dihydro-5H-5λ⁴-thieno[3,2-d]pyrimidin-2-yl]piperidin-4-ol
1.42. {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine
1.43. 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile
1.44. 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl)-(tetrahydropyran-4-yl)-amine
1.45. (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one.

The above mentioned compounds of formula 1 are prepared as described in detail in the synthesis instructions.

Particularly preferred within the scope of the present invention are those of the above mentioned medicament combinations that are selected from among

2.1 and 1.1; 2.1 and 1.2; 2.1 and 1.3, 2.1 and 1.4; 2.1 and 1.5; 2.1 and 1.6; 2.1 and 1.7; 2.1 and 1.8, 2.1 and 1.9; 2.1 and 1.10; 2.1 and 1.11; 2.1 and 1.12; 2.1 and 1.13, 2.1 and 1.14; 2.1 and 1.15; 2.1 and 1.16; 2.1 and 1.17; 2.1 and 1.18, 2.1 and 1.19; 2.1 and 1.20; 2.1 and 1.21; 2.1 and 1.22; 2.1 and 1.23, 2.1 and 1.24; 2.1 and 1.25; 2.1 and 1.26; 2.1 and 1.27; 2.1 and 1.28, 2.1 and 1.29; 2.1 and 1.30; 2.1 and 1.31; 2.1 and 1.32; 2.1 and 1.33, 2.1 and 1.34; 2.1 and 1.35; 2.1 and 1.36; 2.1 and 1.37, 2.1 and 1.38; 2.1 and 1.39; 2.1 and 1.40; 2.1 and 1.41; 2.1 and 1.42, 2.1 and 1.43; 2.1 and 1.44; 2.1 and 1.45;
2.2 and 1.1; 2.2 and 1.2; 2.2 and 1.3, 2.2 and 1.4; 2.2 and 1.5; 2.2 and 1.6; 2.2 and 1.7; 2.2 and 1.8, 2.2 and 1.9; 2.2 and 1.10; 2.2 and 1.11; 2.2 and 1.12; 2.2 and 1.13, 2.2 and 1.14; 2.2 and 1.15; 2.2 and 1.16; 2.2 and 1.17; 2.2 and 1.18, 2.2 and 1.19; 2.2 and 1.20; 2.2 and 1.21; 2.2 and 1.22; 2.2 and 1.23, 2.2 and 1.24; 2.2 and 1.25; 2.2 and 1.26; 2.2 and 1.27; 2.2 and 1.28, 2.2 and 1.29; 2.2 and 1.30; 2.2 and 1.31; 2.2 and 1.32; 2.2 and 1.33, 2.2 and 1.34; 2.2 and 1.35; 2.2 and 1.36; 2.2 and 1.37, 2.2 and 1.38; 2.2 and 1.39; 2.2 and 1.40; 2.2 and 1.41; 2.2 and 1.42, 2.2 and 1.43; 2.2 and 1.44; 2.2 and 1.45;
2.3 and 1.1; 2.1 and 1.3; 2.1 and 1.3, 2.3 and 1.4; 2.3 and 1.5; 2.3 and 1.6; 2.3 and 1.7; 2.3 and 1.8, 2.3 and 1.9; 2.3 and 1.10; 2.3 and 1.11; 2.3 and 1.12; 2.3 and 1.13, 2.3 and 1.14; 2.3 and 1.15; 2.3 and 1.16; 2.3 and 1.17; 2.3 and 1.18, 2.3 and 1.19; 2.3 and 1.20; 2.3 and 1.21; 2.3 and 1.22; 2.3 and 1.23, 2.3 and 1.24; 2.3 and 1.25; 2.3 and 1.26; 2.3 and 1.27; 2.3 and 1.28, 2.3 and 1.29; 2.3 and 1.30; 2.3 and 1.31; 2.3 and 1.32; 2.3 and 1.33, 2.3 and 1.34; 2.3 and 1.35; 2.3 and 1.36; 2.3 and 1.37, 2.3 and 1.38; 2.3 and 1.39; 2.3 and 1.40; 2.3 and 1.41; 2.3 and 1.42, 2.3 and 1.43; 2.3 and 1.44; 2.3 and 1.45;
2.4 and 1.1; 2.4 and 1.2; 2.4 and 1.3, 2.4 and 1.4; 2.4 and 1.5; 2.4 and 1.6; 2.4 and 1.7; 2.4 and 1.8, 2.4 and 1.9; 2.4 and 1.10; 2.4 and 1.11; 2.4 and 1.12; 2.4 and 1.13, 2.4 and 1.14; 2.4 and 1.15; 2.4 and 1.16; 2.4 and 1.17; 2.4 and 1.18, 2.4 and 1.19; 2.4 and 1.20; 2.4 and 1.21; 2.4 and 1.22; 2.4 and 1.23, 2.4 and 1.24; 2.4 and 1.25; 2.4 and 1.26; 2.4 and 1.27; 2.4 and 1.28, 2.4 and 1.29; 2.4 and 1.30; 2.4 and 1.31; 2.4 and 1.32; 2.4 and 1.33, 2.4 and 1.34; 2.4 and 1.35; 2.4 and 1.36; 2.4 and 1.37, 2.4 and 1.38; 2.4 and 1.39; 2.4 and 1.40; 2.4 and 1.41; 2.4 and 1.42, 2.4 and 1.43; 2.4 and 1.44; 2.4 and 1.45;
2.5 and 1.1; 2.5 and 1.2; 2.5 and 1.3, 2.5 and 1.4; 2.5 and 1.5; 2.5 and 1.6; 2.5 and 1.7; 2.5 and 1.8, 2.5 and 1.9; 2.5 and 1.10; 2.5 and 1.11; 2.5 and 1.12; 2.5 and 1.13, 2.5 and 1.14; 2.5 and 1.15; 2.5 and 1.16; 2.5 and 1.17; 2.5 and 1.18, 2.5 and 1.19; 2.5 and 1.20; 2.5 and 1.21; 2.5 and 1.22; 2.5 and 1.23, 2.5 and 1.24; 2.5 and 1.25; 2.5 and 1.26; 2.5 and 1.27; 2.5 and 1.28, 2.5 and 1.29; 2.5 and 1.30; 2.5 and 1.31; 2.5 and 1.32; 2.5 and 1.33, 2.5 and 1.34; 2.5 and 1.35; 2.5 and 1.36; 2.5 and 1.37, 2.5 and 1.38; 2.5 and 1.39; 2.5 and 1.40; 2.5 and 1.41; 2.5 and 1.42, 2.5 and 1.43; 2.5 and 1.44; 2.5 and 1.45;
2.6 and 1.1; 2.6 and 1.2; 2.6 and 1.3, 2.6 and 1.4; 2.6 and 1.5; 2.6 and 1.6; 2.6 and 1.7; 2.6 and 1.8, 2.6 and 1.9; 2.6 and 1.10; 2.6 and 1.11; 2.6 and 1.12; 2.6 and 1.13, 2.6 and 1.14; 2.6 and 1.15; 2.6 and 1.16; 2.6 and 1.17; 2.6 and 1.18, 2.6 and 1.19; 2.6 and 1.20; 2.6 and 1.21; 2.6 and 1.22; 2.6 and 1.23, 2.6 and 1.24; 2.6 and 1.25; 2.6 and 1.26; 2.6 and 1.27; 2.6 and 1.28, 2.6 and 1.29; 2.6 and 1.30; 2.6 and 1.31; 2.6 and 1.32; 2.6 and 1.33, 2.6 and 1.34; 2.6 and 1.35; 2.6 and 1.36; 2.6 and 1.37, 2.6 and 1.38; 2.6 and 1.39; 2.6 and 1.40; 2.6 and 1.41; 2.6 and 1.42, 2.6 and 1.43; 2.6 and 1.44; 2.6 and 1.45;
2.7 and 1.1; 2.7 and 1.2; 2.7 and 1.3, 2.7 and 1.4; 2.7 and 1.5; 2.7 and 1.6; 2.7 and 1.7; 2.7 and 1.8, 2.7 and 1.9; 2.7 and 1.10; 2.7 and 1.11; 2.7 and 1.12; 2.7 and 1.13, 2.7 and 1.14; 2.7 and 1.15; 2.7 and 1.16; 2.7 and 1.17; 2.7 and 1.18, 2.7 and 1.19; 2.7 and 1.20; 2.7 and 1.21; 2.7 and 1.22; 2.7 and 1.23, 2.7 and 1.24; 2.7 and 1.25; 2.7 and 1.26; 2.7 and 1.27; 2.7 and 1.28, 2.7 and 1.29; 2.7 and 1.30; 2.7 and 1.31; 2.7 and 1.32; 2.7 and 1.33, 2.7 and 1.34; 2.7 and 1.35; 2.7 and 1.36; 2.7 and 1.37, 2.7 and 1.38; 2.7 and 1.39; 2.7 and 1.40; 2.7 and 1.41; 2.7 and 1.42, 2.7 and 1.43; 2.7 and 1.44; 2.7 and 1.45;
2.8 and 1.1; 2.8 and 1.2; 2.8 and 1.3, 2.8 and 1.4; 2.8 and 1.5; 2.8 and 1.6; 2.8 and 1.7; 2.8 and 1.8, 2.8 and 1.9; 2.8 and 1.10; 2.8 and 1.11; 2.8 and 1.12; 2.8 and 1.13, 2.8 and 1.14; 2.8 and 1.15; 2.8 and 1.16; 2.8 and 1.17; 2.8 and 1.18, 2.8 and 1.19; 2.8 and 1.20; 2.8 and 1.21; 2.8 and 1.22; 2.8 and 1.23, 2.8 and 1.24; 2.8 and 1.25; 2.8 and 1.26; 2.8 and 1.27; 2.8 and 1.28, 2.8 and 1.29; 2.8 and 1.30; 2.8 and 1.31; 2.8 and 1.32; 2.8 and 1.33, 2.8 and 1.34; 2.8 and 1.35; 2.8 and 1.36; 2.8 and 1.37, 2.8 and 1.38; 2.8 and 1.39; 2.8 and 1.40; 2.8 and 1.41; 2.8 and 1.42, 2.8 and 1.43; 2.8 and 1.44; 2.8 and 1.45.

Also particularly preferred is one of the above medicament combinations in which the PDE4 inhibitor (1) is contained in a daily dose of 0.01 mg to 50 mg, preferably 0.1 mg to 10 mg.

in another particularly preferred aspect the invention relates to one of the above mentioned medicament combinations, in which the EP4-receptor-antagonist (2) is used in a daily dose of 0.001 to 100 mg/kg body weight, preferably 0.01 to 50 mg/kg body weight, more preferably 0.1 to 10 mg/kg body weight.

Also particularly preferred is one of the above medicament combinations wherein the EP4-receptor antagonist (2) and the PDE4 inhibitor (1) are used in a ratio by weight of 1:1 to 200:1, preferably in a ratio by weight of 10:1 to 150:1, particularly preferably in a ratio by weight of 30:1 to 100:1.

The present invention further relates to the use of an EP4-receptor-antagonist (2) for reducing the side effects of one or more PDE4-inhibitors in the treatment of a disease selected from among respiratory complaints, pulmonary diseases, gastrointestinal complaints, diseases such as inflammatory diseases of the joints, skin or eyes, cancers and diseases of the peripheral or central nervous system.

The present invention further relates to the use of a combination containing one or more PDE4-inhibitors (1) and at least one EP4 receptor antagonist (2) for the treatment of a disease selected from among respiratory complaints, pulmonary diseases, gastrointestinal complaints, diseases such as inflammatory diseases of the joints, skin or eyes, cancers and diseases of the peripheral or central nervous system.

In another preferred aspect the present invention relates to one of the above mentioned uses, wherein the or each PDE4-inhibitor is a compound of general formula 1,

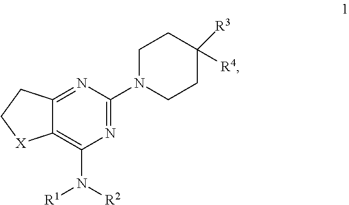

wherein X, R¹, R², R³ and R⁴ are as hereinbefore defined.

The present invention further relates to the above-mentioned uses of an EP4-receptor-antagonist (2) or a combination containing one or more PDE4-inhibitors (1) and at least one EP4 receptor antagonist (2) for the treatment of a disease selected from COPD, chronic sinusitis, asthma, Crohn's disease and ulcerative colitis.

In a variant of the above-mentioned uses the PDE4 inhibitor (1) and the at least one EP4-receptor antagonist (2) are administered simultaneously in a single common formulation.

In another variant of the above-mentioned uses the PDE4 inhibitor (1) and the at least one EP4-receptor antagonist (2) may, however, also be administered in two separate formulations offset from one another within a time interval of 0 to 6 hours.

In this separate administration in two separate formulations the formulation containing the PDE4 inhibitor—particularly the PDE4 inhibitor of formula 1—may be an oral or inhalative formulation, but is preferably an oral formulation, and the formulation containing the at least one EP4 receptor antagonist (2) is preferably an oral formulation.

Moreover, when the combination is used in separate formulations for preparing a medicament combination for the treatment of the above mentioned diseases the formulation containing the PDE4 inhibitor—particularly the PDE4 inhibitor of formula 1—is preferably administered once a day and the formulation containing the at least one EP4 receptor antagonist (2) is preferably administered either once or twice a day.

In particular, in the above mentioned uses the PDE4-inhibitors (1) of formula 1 are selected from:
1.1. (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol
1.2. (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol
1.3. (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol
1.4. (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol
1.5. (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one
1.6. {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine
1.7. 1-(4-(1-hydroxymethylcyclopropylamino)-5-oxo-6,7-dihydro-5H-5λ⁴-thieno[3,2-d]pyrimidin-2-yl)-3′-methyl-1′H-spiro[piperidin-4,4′-quinazolin]-2′(37-1)-one
1.8. {1-[2-(4-benzo[d]isoxazol-3-yl-piperidin-1-yl)-5-oxo-6,7-dihydro-5H-5λ⁴-thieno[3,2-d]pyrimidin-4-ylamino]-cyclopropyl}-methanol
1.9. (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol
1.10. 1-[4-((S)-1-methyl-6-oxopiperidin-3-ylamino)-5-oxo-6,7-dihydro-5H-5λ⁴-thieno[3,2-d]pyrimidin-2-yl]-4-phenylpiperidin-4-carbonitrile
1.11. 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(37-1)-one
1.12. (3-fluorophenyl)-[5-oxo-2-(3,4,5,6-tetrahydro-2H-[4,4′]bipyridinyl-1-yl)-6,7-dihydro-5H-5λ⁴-thieno[3,2-d]pyrimidin-4-yl]-amine
1.13. {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine
1.14. (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol
1.15. {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine
1.16. (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one
1.17. (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.18. (1-{2-[4-(5-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.19. {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.20. (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine
1.21. (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol
1.22. (S)-5-{2-[4-(4-fluorophenoxy)-piperidin-1-yl]-5-oxo-6,7-dihydro-5H-5λ⁴-thieno[3,2-d]pyrimidin-4-ylamino}-1-methylpiperidin-2-one
1.23. (2-{4-[4-(4,5-dihydroxazol-2-yl)-phenoxyl]-piperidin-1-yl}-5-oxo-6,7-dihydro-5H-5λ⁴-thieno[3,2-d]pyrimidin-4-yl)-(tetrahydropyran-4-yl)-amine
1.24. 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid
1.25. 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol
1.26. {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine
1.27. 2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl)-(tetrahydropyran-4-yl)-amine
1.28. (S)-5-(2-{4-[4-(4,5-dihydroxazol-2-yl)-phenoxyl]-piperidin-1-yl}-5-oxo-6,7-dihydro-5H-5λ⁴-thieno[3,2-d]pyrimidin-4-ylamino)-1-methylpiperidin-2-one
1.29. {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.30. {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine
1.31. 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide
1.32. N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide
1.33. N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yloxy}-benzamide
1.34. {5-oxo-2-[4-(pyridin-4-yloxy)-piperidin-1-yl]-6,7-dihydro-5H-5λ⁴-thieno[3,2-d]pyrimidin-4-yl}-(tetrahydropyran-4-yl)-amine
1.35. {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine
1.36. (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one
1.37. (1-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-cyclopropyl)-methanol
1.38. (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one
1.39. {4-(4-chlorophenyl)-1-[5-oxo-4-(tetrahydropyran-4-ylamino)-6,7-dihydro-5H-5λ⁴-thieno[3,2-d]pyrimidin-2-yl]piperidin-4-yl}-methanol
1.40. [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol
1.41. 4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-ol
1.42. {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine
1.43. 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile
1.44. 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl}-(tetrahydropyran-4-yl)-amine
1.45. (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one.

In these above mentioned uses the EP4-receptor-antagonists (2) are preferably selected from among:
[N-{[4-(5,9-diethoxy-6-oxo-6,8-dihydro-7H-pyrrolo[3,4-g]quinolin-7-yl)-3-methylbenzyl]sulphonyl}-2-(2-methoxyphenyl)acetamide] (2.1);
5-butyl-2,4-dihydro-4-[[2′[N-(3-methyl-2-thiophene-carbonyl)sulphamoyl]biphenyl-4-yl]methyl]-2-[(2-trifluoromethyl)phenyl]-1,2,4-triazol-3-one (2.2);
(4-{(1S)-1-[({5-chloro-2-[(4-fluorophenyl)oxy]phenyl}carbonyl)amino]ethyl}benzoic acid (2.3),
N-[({2-[4-(2-ethyl-4,6-dimethyl-1H-imidazo[4,5-c]pyridin-1-yl)phenyl]ethyl}amino)carbonyl]-4-methylbenzene sulphonamide (2.4);
4-[[4-(5-methoxy-2-pyridinyl)phenoxy]methyl]-5-methyl-N-[(2-methylphenyl)sulphonyl]-2-furancarboxamide (2.5);
methyl 11alpha, 15alpha-dihydroxy-16-(3-methoxymethylphenyl)-9-oxo-17,18,19,20-tetranor-5-thia-13(E)prostanoate (2.6);
4-cyano-2-[[2-(4-fluoro-1-naphthalenyl)-1-oxopropyl]amino]-benzenebutanoic acid (2.7) and
N-{2-[4-(4,9-diethoxy-1-oxo-1,3-dihydro-2H-benzo[f]isoindol-2-yl)phenyl]acetyl}benzene sulphonamide (2.8).

In a particularly preferred embodiment of the invention, in these above mentioned uses the PDE4 inhibitor of general formula 1 is used in a daily dose of 0.01 mg to 50 mg.

In another particularly preferred embodiment of the invention in these above mentioned uses the EP4-receptor antagonist (2) is used in a daily dose of 0.001 to 100 mg/kg body weight, preferably in a daily dose of 0.01 to 50 mg/kg body weight, more preferably in a daily dose of 0.1 to 10 mg/kg body weight.

In another particularly preferred embodiment of the invention in these above mentioned uses the EP4-receptor antagonist (2) and the PDE4 inhibitor (1) are used in a ratio by weight of 1:1 to 200:1, preferably in a ratio by weight of 10:1 to 150:1, particularly preferably in a ratio by weight of 30:1 to 100:1.

In particular, the invention relates to the above mentioned uses, wherein the or at least one or more of the PDE4 inhibitor-mediated side effects in considerably reduced or totally prevented, compared with the sole administration of the PDE4 inhibitor used in the medicament combination.

In particular, the invention further relates to the use of EP4-receptor-antagonists, preferably as hereinbefore defined and according to the preferred definition, for reducing or preventing one or more PDE4 inhibitor-mediated side effects.

These PDE4 inhibitor-mediated side effects are preferably selected from loss of body weight, spleen weight loss, leukocytosis, neutrophilia, nausea, vomiting, diarrhoea and mesenteric vasculitis. These PDE4 inhibitor-mediated side effects are more preferably selected from loss of body weight, spleen weight loss, leukocytosis, neutrophilia and mesenteric vasculitis.

### SYNTHESIS INSTRUCTIONS

The compounds of general formula (I) may be prepared according to the following general synthesis scheme, wherein the substituents of general formula (I) have the meanings given hereinbefore. These methods are to be understood as being an explanation of the invention without restricting it to their subject-matter.

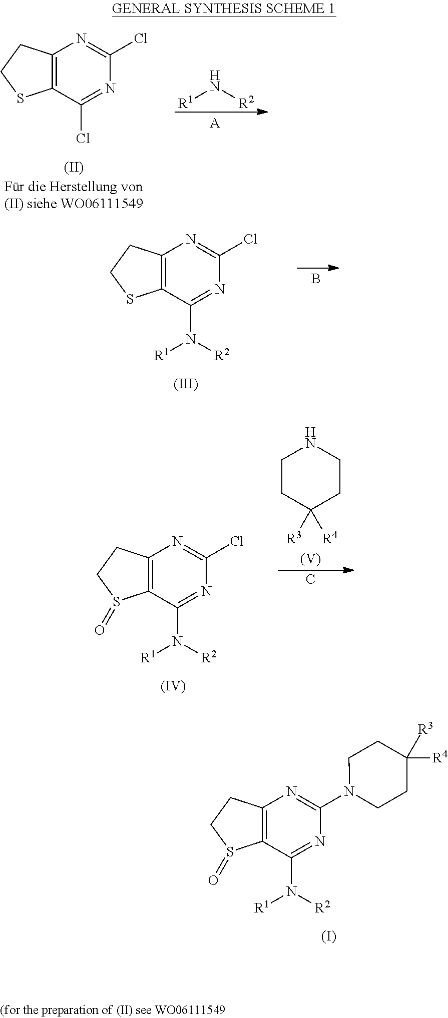

### 1. Synthesis of (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol Example 1.1

### 1.1 (R)-2-(2-chloro-6,7-dihydrothieno[3,2-d]pyrimidin-4-ylamino)-3-methylbutan-1-ol (III-1)

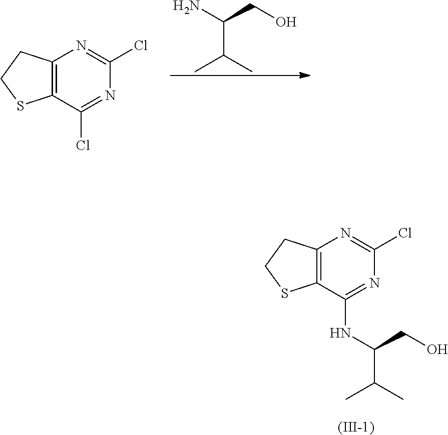

7.2 g 2,4-dichloro-6,7-dihydrothieno[3,2-d]pyrimidine (II) are placed in 36 ml dioxane, first 18 ml diisopropylethylamine, then 6.1 g (R)-(−)-2-amino-3-methyl-1-butanol are added. The reaction mixture is heated to 100° C. until there is no further reaction, and after cooling evaporated down. The residue is treated with petroleum ether/ethyl acetate (9:1) in the ultrasound bath and the solid is suction filtered and dried. 8.3 g (III-1) are obtained as a solid. Analytical HPLC (method A): RT=2.75 min

### 1.2 (R)-2-(2-chloro-5-oxo-6,7-dihydro-5H-5λ⁴-thieno[3,2-d]pyrimidin-4-ylamino)-3-methylbutan-1-ol (IV-1)

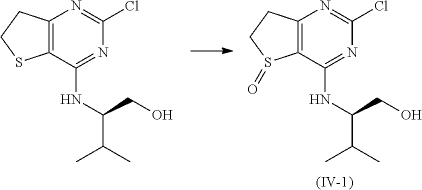

4.1 g S-(−)-1,1′-bi-2-naphthol are placed under argon in 15 ml chloroform, then 0.44 ml titanium(IV)isopropoxide and 0.54 ml of water are added. The reaction mixture is stirred for 1 hour at ambient temperature. Then a suspension of 4.1 g (III-1) in 107 ml dichloromethane is added. The reaction mixture is cooled to −2° C. and after 30 minutes 2.7 ml tert-butylhydroperoxide 5-6 M in decane are added dropwise. The reaction mixture is stirred further at −2° C. until there is no further reaction, and made basic with NH₄OH. The product is extracted with dichloromethane and purified by chromatography (silica gel, ethyl acetate/methanol 100/0 to 86/14). 2.45 g (IV-1) are obtained as a solid. Analytical HPLC (method A): RT=2.37 min

### 1.3 (R)-2-{2-[4-(4-chlorophenyl)-piperidin-1-yl]-5-oxo-6,7-dihydro-5H-5λ⁴-thieno[3,2-c]pyrimidin-4-ylamino}-3-methylbutan-1-ol (Example 1.1)

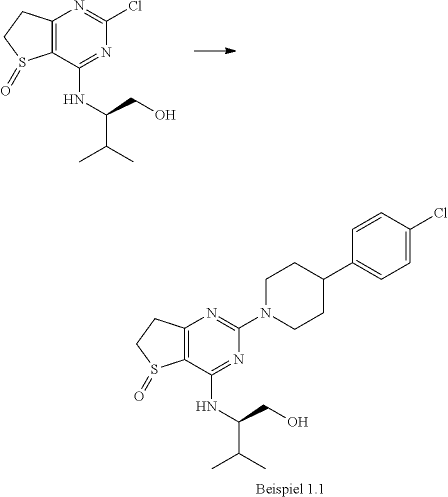

0.2 g (IV-1) is placed in 3 ml dioxane and 360 μl diisopropylethylamine, combined with 0.16 g 4-(4-chlorophenyl)-piperidine and heated to 120° C. in the microwave until there is no further reaction. The reaction mixture is mixed with water, extracted with dichloromethane and the product is purified by chromatography (silica gel, dichloromethane/methanol 100/0 to 92/8). 0.33 g Example 1.1 are obtained as a solid. Analytical HPLC-MS (method A): RT=1.24 min.

### 2. Synthesis of (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol (Example 1.2)

### 2.1 tert-butyl(1-hydroxymethylcyclopropyl)-carbamidate

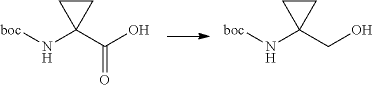

1 g 1-(BOC-amino)-cyclopropanecarboxylic acid is dissolved in 20 ml dimethoxyethane and cooled to −70° C. Then 0.65 ml N-methylmorpholine are added and 0.71 ml isobutylchloroformate in 5 ml dimethoxyethane are added dropwise. The reaction mixture is heated to −5° C. The precipitate is suction filtered. The eluate is cooled to −15° C. and 0.303 g sodium borohydride are slowly added. The reaction mixture is then stirred for 30 minutes at ambient temperature, mixed with water and the product is extracted with dichloromethane. The organic phase is dried and evaporated to dryness. 1.04 g product are obtained as a solid. ¹H NMR (400 MHz, DMSO): 1.36 (9H, s); 0.61 (2H, t); 0.52 (2H, t).

### 2.2 1-aminocyclopropanmethanol

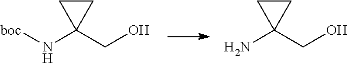

1.04 g tert-butyl (1-hydroxymethylcyclopropyl)-carbamidate are placed in 5 ml dioxane. 2.5 ml HCl in dioxane (4 mol/l) are added dropwise. The reaction mixture is stirred for 15 h at ambient temperature. The solvent is evaporated down by half and the precipitated solid is suction filtered. 0.5 g product are obtained as the hydrochloride. ¹H NMR (400 MHz, DMSO): 5.27 (1H, t); 0.91 (2H, t); 0.71 (2H, t).

### 2.3 [1-(2-chloro-6,7-dihydrothieno[3,2-d]pyrimidin-4-ylamino)-cyclopropyl]-methanol (III-2)

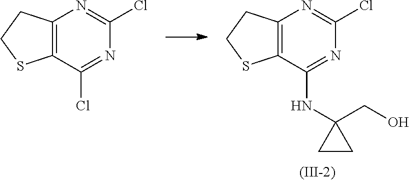 [SMILES: Clc1nc(Cl)c2c(n1)CCS2 ; OCC1(Nc2nc(Cl)nc3c2SCC3)CC1 ; "(III-2)"]

1.4 g (II) are placed in 10 ml dioxane, then 3.6 ml diisopropylethylamine and then 1 g of 1-aminocyclopropanemethanol (see 2.2) are added. The reaction mixture is heated to 160° C. until there is no further reaction, and after cooling evaporated down. The residue is treated with cyclohexane/ethyl acetate (4:1) in the ultrasound bath, the solid is suction filtered and dried. 1.24 g (III-2) are obtained as a solid. Analytical HPLC-MS (method A): RT=1.01 min.

### 2.4 [1-(2-chloro-5-oxo-6,7-dihydro-5H-5λ⁴-thieno[3,2-d]pyrimidin-4-ylamino)-cyclopropyl]-methanol (IV-2)

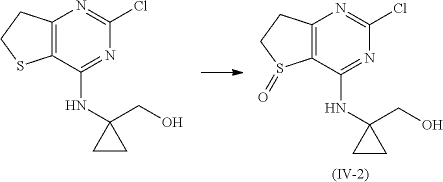

0.28 g S-(−)-1,1′-bi-2-naphthol are placed in 20 ml chloroform under argon, then 0.14 ml titanium(IV)isopropoxide and 0.17 ml of water are added. The reaction mixture is stirred for 1 hour at ambient temperature. Then a suspension of 1.2 g (III-2) in 40 ml dichloromethane and 2 ml of methanol is added. The reaction mixture is cooled to −5° C. and after 30 minutes 0.91 ml tert-butylhydroperoxide 5-6 M in decane are added dropwise. The reaction mixture is stirred further at −5° C. until there is no further reaction, and made basic with NH₄OH. The aqueous phase is washed with dichloromethane and freeze-dried. 1 g (IV-2) is obtained as a solid. Analytical HPLC-MS (method A) RT=0.85 min

### 2.5 (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol (Example 1.2)

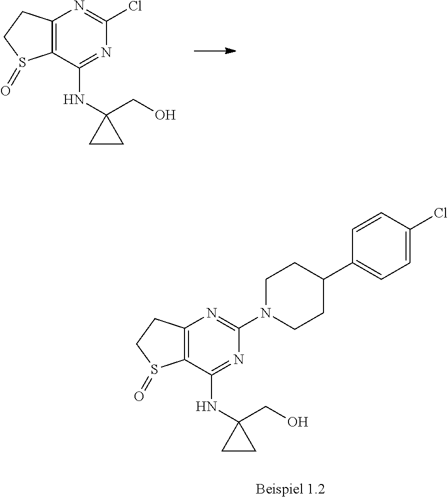

Starting from 0.17 g (IV-2) and 0.15 g 4-(4-chlorophenyl)-piperidine 0.14 g Example 1.2 are prepared and purified analogously to Example 1.1 (see 1.3). Analytical HPLC-MS (method B): RT=1.32 min.

### 3. Synthesis of (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol (Example 1.3)

### 3.1 (R)-2-(2-chloro-6,7-dihydrothieno[3,2-d]pyrimidin-4-ylamino)-pentan-1-ol (III-3)

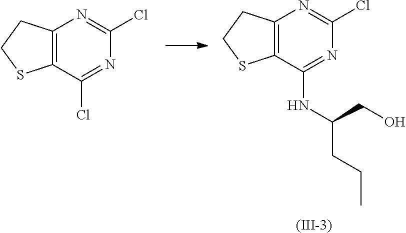 [SMILES: Clc1nc(Cl)c2c(n1)CCS2 ; CCC[C@H](CO)Nc1nc(Cl)nc2c1SCC2 ; "(III-3)"]

1.4 g 2,4-dichloro-6,7-dihydrothieno[3,2-d]pyrimidine (II) are placed in 9 ml dioxane, first 3.5 ml diisopropylethylamine, then 0.9 g D-norvalinol are added. The reaction mixture is heated to 120° C. in the microwave until there is no further reaction and after cooling it is evaporated down. The residue is treated with petroleum ether/ethyl acetate 9:1 in the ultrasound bath, the solid is suction filtered and dried. 1.5 g (III-3) are obtained as a solid. ¹H NMR (400 MHz, DMSO): 4.67 (1H, t); 0.86 (3H, t).

### 3.2 (R)-2-(2-chloro-5-oxo-6,7-dihydro-5H-5λ⁴-thieno[3,2-d]pyrimidin-4-ylamino)-pentan-1-ol (IV-3)

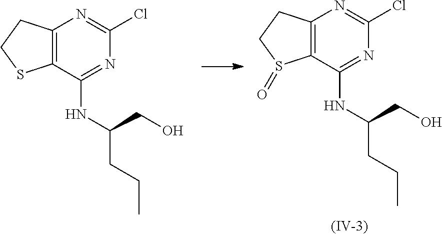 [SMILES: CCC[C@H](CO)Nc1nc(Cl)nc2c1SCC2 ; CCC[C@H](CO)Nc1nc(Cl)nc2c1S(=O)CC2 ; "(IV-3)"]

0.3 g S-(−)-1,1′-bi-2-naphthol are placed in 5 ml chloroform under argon, then 0.15 ml titanium(IV)isopropoxide and 0.19 ml of water are added. The reaction mixture is stirred for 1 hour at ambient temperature. Then a suspension of 1.4 g (III-3) in 20 ml dichloromethane is added. The reaction mixture is cooled to −5° C. and after 30 minutes 0.95 ml tert-butylhydroperoxide 5-6 M in decane are added dropwise. The reaction mixture is stirred further at −5° C. until there is no further reaction, and made basic with NH₄OH. The product is extracted with dichloromethane and purified by chromatography (ethyl acetate/methanol 100/0 to 80/20). 1.17 g (IV-3) are obtained as a solid. Analytical HPLC (method A): RT=2.41 min

### 3.3 (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol (Example 1.3)

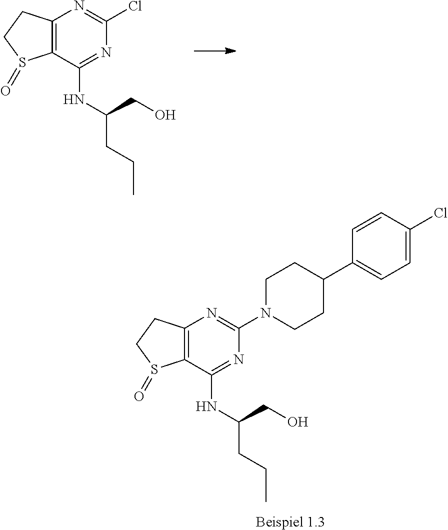

0.2 g (IV-3) are placed in 4 ml dioxane and 237 μl diisopropylethylamine, mixed with 0.149 g 4-(4-chlorophenyl)-piperidine and heated to 130° C. in the microwave for 30 min. The reaction mixture is mixed with water and the product is extracted with dichloromethane. The residue is treated with acetonitrile in the ultrasound bath and the solid suction filtered. 0.104 g Example 1.3 are obtained as a solid. Analytical HPLC-MS (method A): RT=1.29 min.

### 4. Synthesis of (R)-1-{2-[4-(4-chlorophenyl)-piperidin-1-yl]-5-Oxo-6,7-dihydro-5H-5λ⁴-thieno[3,2-d]pyrimidin-4-ylamino}-1-(4-fluorophenyl)-2-methylpropan-2-ol (Example 1.4)

### 4.1 methyl(R)-amino-(4-fluorophenyl)-acetate

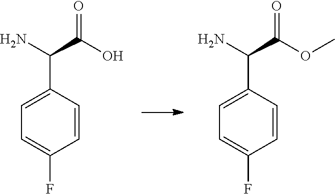

4 g (R)-4-fluorophenylglycine are suspended in 80 ml of methanol. While cooling with the ice bath 3.28 ml of thionyl chloride are slowly added dropwise, so that the temperature is held between 15° C. and 20° C. The reaction mixture is stirred for 12 hours at ambient temperature and then evaporated to dryness. 5.1 g of the product are obtained as the hydrochloride. Analytical HPLC-MS (method A): RT=0.8 min.

### 4.2 methyl(R)-(4-fluorophenyl)-(2,2,2-trifluoracetylamino)-acetate

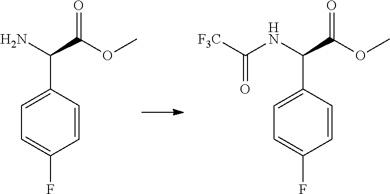 [SMILES: COC(=O)[C@H](N)c1ccc(F)cc1 ; COC(=O)[C@H](NC(=O)C(F)(F)F)c1ccc(F)cc1]

5.1 g methyl(R)-amino-(4-fluorophenyl)-acetate are placed in 36.5 ml abs. tetrahydrofuran, then 3.9 ml triethylamine are added. The reaction mixture is cooled to −70° C. 3.9 ml trifluoroacetic anhydride are then slowly added dropwise, so that the temperature does not exceed −60° C. The reaction mixture is stirred for 12 hours at ambient temperature and then mixed with water. Then potassium hydrogen carbonate is added until no more foaming can be observed, and the product is extracted with ethyl acetate. 6.2 g of the product are obtained as an oil. Analytical HPLC-MS (method A): RT=1.28 min.

### 4.3 2,2,2-trifluoro-N—[(R)-1-(4-fluorophenyl)-2-hydroxy-2-methylpropyl]-acetamide

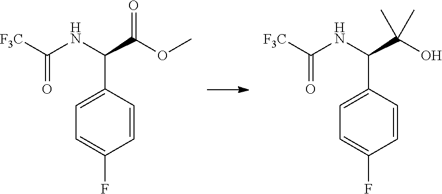 [SMILES: COC(=O)[C@H](NC(=O)C(F)(F)F)c1ccc(F)cc1 ; CC(C)(O)[C@H](NC(=O)C(F)(F)F)c1ccc(F)cc1]

6.2 g methyl(R)-(4-fluorophenyl)-(2,2,2-trifluoracetylamino)-acetate are placed in 195 ml abs. tetrahydrofuran and the reaction mixture is cooled to +3° C. 37.2 ml of a methylmagnesium iodide solution (3 M) are slowly added dropwise, so that the temperature does not exceed +10° C. The reaction mixture is stirred for 12 hours at ambient temperature and then stirred in ice water. Ammonium chloride is added until the precipitate is dissolved and the product is extracted with ethyl acetate. 5.6 g of the product is obtained as an oil. Analytical HPLC-MS (method A): RT=1.19 min

### 4.4 (R)-1-amino-1-(4-fluorophenyl)-2-methylpropan-2-ol

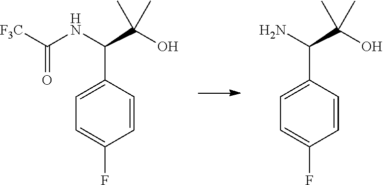 [SMILES: CC(C)(O)[C@H](NC(=O)C(F)(F)F)c1ccc(F)cc1 ; CC(C)(O)[C@H](N)c1ccc(F)cc1]

5.6 g 2,2,2-trifluoro-N—[(R)-1-(4-fluorophenyl)-2-hydroxy-2-methylpropyl]-acetamide and 2.27 g KOH are suspended in 60 ml of methanol. The reaction mixture is stirred for 20 hours at 60° C., then mixed with water and the product is extracted with dichloromethane. 3.2 g product are obtained as an oil. Analytical HPLC-MS (method A): RT=0.79 min.

### 4.5 (R)-1-(2-chloro-6,7-dihydrothieno[3,2-d]pyrimidin-4-ylamino)-1-(4-fluorophenyl)-2-methylpropan-2-ol (III-4)

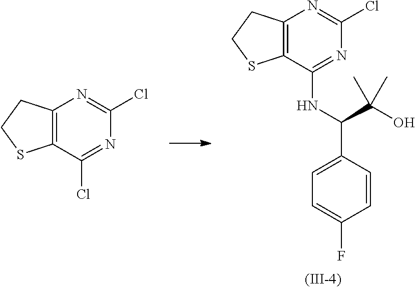 [SMILES: Clc1nc(Cl)c2c(n1)CCS2 ; CC(C)(O)[C@H](Nc1nc(Cl)nc2c1SCC2)c1ccc(F)cc1 ; "(III-4)"]

0.533 g (II), 0.850 g (R)-1-amino-1-(4-fluorophenyl)-2-methylpropan-2-ol and 1.3 ml diisopropylethylamine are suspended in 9.8 ml dioxane. The reaction mixture is heated in the microwave for 2 hours at 80° C. and then evaporated to dryness. The residue is mixed with water. The precipitate formed is suction filtered and purified by chromatography (silica gel, petroleum ether/ethyl acetate 100/0 to 60/40). 0.260 g (III-4) are obtained as a solid. Analytical HPLC-MS (method A): 1.39 min.

### 4.6 (R)-1-(2-chloro-5-oxo-6,7-dihydro-5H-5λ⁴-thieno[3,2-d]pyrimidin-4-ylamino)-1-(4-fluorophenyl)-2-methylpropan-2-ol (IV-4)

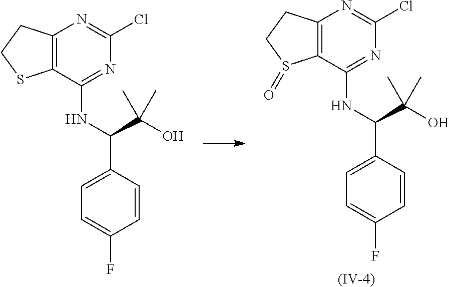 [SMILES: CC(C)(O)[C@H](Nc1nc(Cl)nc2c1SCC2)c1ccc(F)cc1 ; CC(C)(O)[C@H](Nc1nc(Cl)nc2c1S(=O)CC2)c1ccc(F)cc1 ; "(IV-4)"]

0.24 g S-(−)-1,1′-bi-2-naphthol are placed in 4 ml chloroform under argon, then 0.125 ml titanium(IV)isopropoxide and 0.15 ml of water are added. The reaction mixture is stirred for 1 hour at ambient temperature. Then a suspension of 1.51 g (III-4) in 26 ml chloroform is added. The reaction mixture is cooled to −6° C. and after 30 minutes 0.78 ml tert-butylhydroperoxide 5-6 M in decane are added dropwise. The reaction mixture is stirred further at −6° C. until there is no further reaction, and made basic with NH₄OH. The product is extracted with dichloromethane and purified by chromatography (dichloromethane/methanol 100/0 to 95/5). 0.62 g (IV-4) are obtained as a solid. Analytical HPLC-MS (method A): RT=1.19 min.

### 4.7 (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol (Example 1.4)

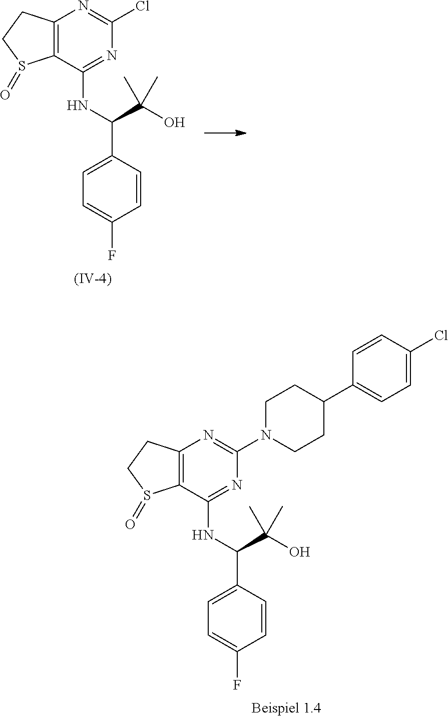

Starting from 0.24 g (IV-4) and 0.15 g 4-(4-chlorophenyl)-piperidine 0.19 g Example 1.4 are prepared analogously to Example 1.1 (see 1.3). The product is purified by chromatography (dichloromethane/methanol 100/0 to 96/4). Analytical HPLC-MS (method A): RT=1.36 min.

### 5. Synthesis of (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-One (Example 1.5)

### 5.1 (S)-5-dibenzylaminopiperidin-2-one

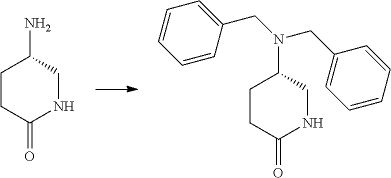 [SMILES: N[C@H]1CCC(=O)NC1 ; O=C1CC[C@H](N(Cc2ccccc2)Cc2ccccc2)CN1]

0.600 g 4-(S)-amino-delta-valerolactam hydrochloride, 0.970 ml benzylbromide and 1.5 g sodium hydrogen carbonate are suspended in 30 ml of ethanol. The reaction mixture is then stirred for 8 hours at 80° C. and then evaporated to dryness. The residue is suspended in water and the product is extracted with dichloromethane and purified by chromatography (silica gel, dichloromethane/methanol 100/0 to 95/5). 0.500 g product are obtained as an oil. Analytical HPLC-MS (method A): RT=1.01 min.

### 5.2 (S)-5-dibenzylamino-1-methylpiperidin-2-one

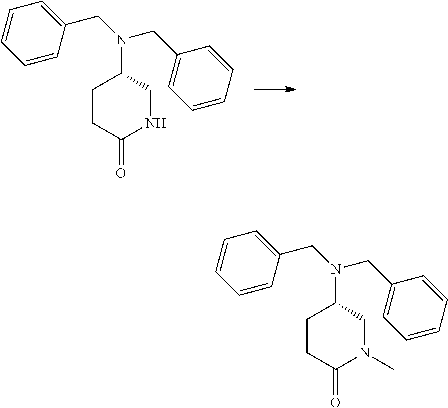 [SMILES: O=C1CC[C@H](N(Cc2ccccc2)Cc2ccccc2)CN1 ; CN1C[C@@H](N(Cc2ccccc2)Cc2ccccc2)CCC1=O]

0.500 g (S)-5-dibenzylaminopiperidin-2-one are suspended in 15 ml of tetrahydrofuran. While cooling with the ice bath 0.175 g potassium-tert-butoxide are added. The reaction mixture is then stirred for 30 minutes at ambient temperature. While cooling with the ice bath 0.095 ml methyl iodide are added. The reaction mixture is then stirred for 48 hours at ambient temperature and then combined with a saturated NaCl solution. The product is extracted with ethyl acetate. 0.450 g product are obtained as an oil. Analytical HPLC-MS (method A): RT=1.07 min.

### 5.3 (S)-5-amino-1-methylpiperidin-2-one

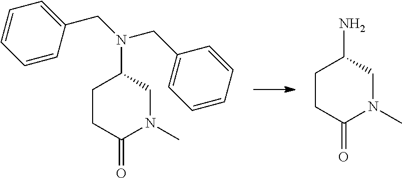

0.450 g (S)-5-dibenzylamino-1-methylpiperidin-2-one are suspended in 25 ml of methanol and hydrogenated with 0.150 g Pd/C 10% at a pressure of 3 bar and a temperature of 60° C. After 16 hours the catalyst is suction filtered and the filtrate is evaporated to dryness. 0.190 g of the product are obtained as an oil. ¹H NMR (400 MHz, DMSO): 2.76 (3H, s).

### 5.4 (S)-5-(2-chloro-6,7-dihydrothieno[3,2-d]pyrimidin-4-ylamino)-1-methylpiperidin-2-one (III-5)

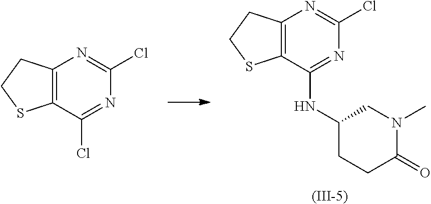 [SMILES: Clc1nc(Cl)c2c(n1)CCS2 ; CN1C[C@@H](Nc2nc(Cl)nc3c2SCC3)CCC1=O ; "(III-5)"]

0.27 g (II) are placed in 3 ml dioxane, then first 0.45 ml diisopropylethylamine, then 0.25 g (S)-5-amino-1-methylpiperidin-2-one are added. The reaction mixture is heated to 130° C. until there is no further reaction and after cooling evaporated down. The product is extracted with dichloromethane and purified by chromatography (preparative HPLC, method A). 0.26 g (III-5) are obtained as a solid. Analytical HPLC-MS (method A): RT=1.06 min.

### 5.5 (S)-5-(2-chloro-5-oxo-6,7-dihydro-5H-5λ⁴-thieno[3,2-d]pyrimidin-4-ylamino)-1-methylpiperidin-2-one (IV-5)

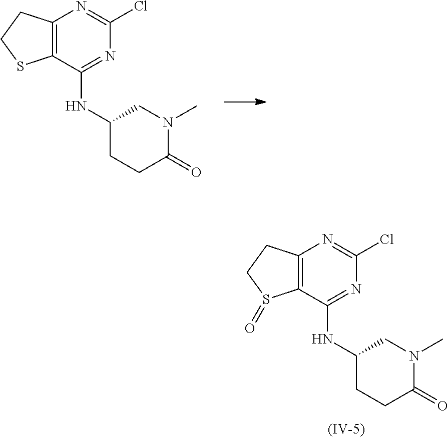 [SMILES: CN1C[C@@H](Nc2nc(Cl)nc3c2SCC3)CCC1=O ; CN1C[C@@H](Nc2nc(Cl)nc3c2S(=O)CC3)CCC1=O ; "(IV-5)"]

0.04 g S-(−)-1,1′-bi-2-naphthol are placed in 5 ml chloroform under argon, then 0.02 ml titanium(IV)isopropoxide and 0.025 ml of water are added. The reaction mixture is stirred for 1 hour at ambient temperature. Then a suspension of 0.2 g (III-5) in 4 ml dichloromethane is added. The reaction mixture is cooled to −5° C. and after 20 minutes 0.12 ml tert-butylhydroperoxide 5-6 M in decane are added dropwise. The reaction mixture is stirred further at −5° C. until there is no further reaction, and made basic with NH₄OH. The product is purified by chromatography (silica gel, ethyl acetate/methanol 100/0 to 60/40). 0.09 g (IV-5) are obtained as a solid. Analytical HPLC-MS (method A): RT=0.83 min.

### 5.6 (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one (Example 1.5)

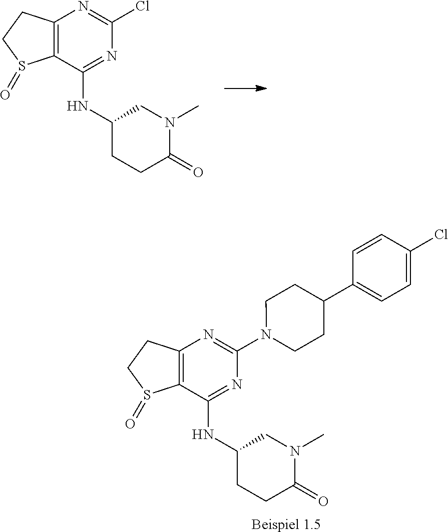

Starting from 0.2 g (IV-5) and 0.18 g 4-(4-chlorophenyl)-piperidine 0.17 g Example 1.5 are prepared analogously to Example 1.1 (see 1.3). The product is purified by chromatography (preparative HPLC, method A). The product fractions are made basic with ammonia and freeze-dried. Analytical HPLC-MS (method A): RT=1.18 min

### 6. Synthesis of {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine (Example 1.6)

### 6.1 (2-chloro-6,7-dihydrothieno[3,2-d]pyrimidin-4-yl)-(tetrahydropyran-4-yl)-amine (III-6)

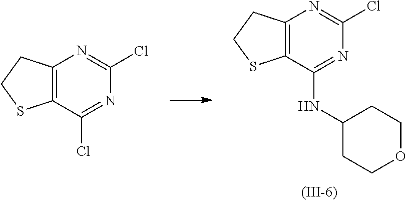 [SMILES: Clc1nc(Cl)c2c(n1)CCS2 ; Clc1nc2c(c(NC3CCOCC3)n1)SCC2 ; "(III-6)"]

0.68 g (II) are placed in 6 ml dioxane, first 1.72 ml diisopropylethylamine, then 0.6 g 4-aminotetrahydropyran are added. The reaction mixture is heated to 130° C. until there is no further reaction and after cooling it is evaporated down. The product is treated with water in the ultrasound bath, then suction filtered and dried. 0.66 g (III-6) are obtained as a solid. Analytical HPLC-MS (method C): RT=1.08 min.

### 6.2 (2-chloro-5-oxo-6,7-dihydro-5H-5λ⁴-thieno[3,2-d]pyrimidin-4-yl)-(tetrahydropyran-4-yl)-amine (IV-6)

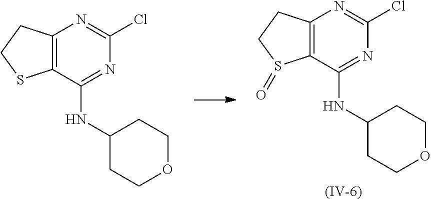 [SMILES: Clc1nc2c(c(NC3CCOCC3)n1)SCC2 ; O=S1CCc2nc(Cl)nc(NC3CCOCC3)c21 ; "(IV-6)"]

0.14 g S-(−)-1,1′-bi-2-naphthol are placed in 5 ml chloroform under argon, then 0.072 ml titanium(IV)isopropoxide and 0.087 ml of water are added. The reaction mixture is stirred for 45 minutes at ambient temperature. Then a suspension of 0.66 g (III-6) in 25 ml chloroform is added. The reaction mixture is cooled to −10° C. and after 60 minutes 0.444 ml tert-butylhydroperoxide 5-6 M in decane are added dropwise. The reaction mixture is stirred further at −10 to −4° C. until there is no further reaction, and mixed with water. The product is extracted with dichloromethane and purified by chromatography (silica gel, ethyl acetate/methanol 100/0 to 80/20). 0.42 g (IV-6) are obtained as a solid. Analytical HPLC-MS (method A): RT=0.94 min.

### 6.3 {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine (Example 1.6)

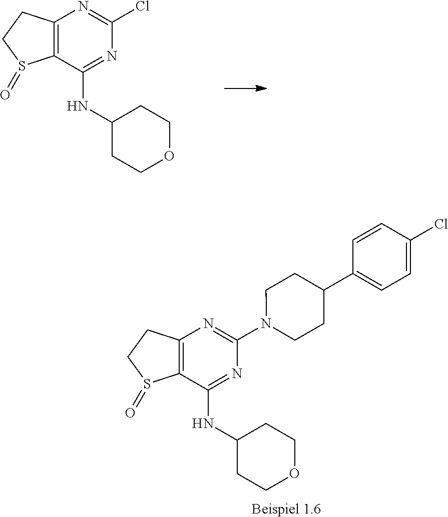

Starting from 0.18 g (IV-6) and 0.17 g 4-(4-chlorophenyl)-piperidine 0.23 g Example 1.6 are prepared analogously to Example 1.1 (see 1.3). The product is treated with water in the ultrasound bath and the solid is suction filtered. Analytical HPLC-MS (method A): RT=1.24 min

### 7. Synthesis of 1-(4-(1-hydroxymethylcyclopropylamino)-5-oxo-6,7-dihydro-5H-5λ⁴-thienO[3,2-d]pyrimidin-2-yl)-3′-methyl-1′H-spiro[piperidin-4,4′-quinazolin]-2′(3′H)-one (Example 1.7)

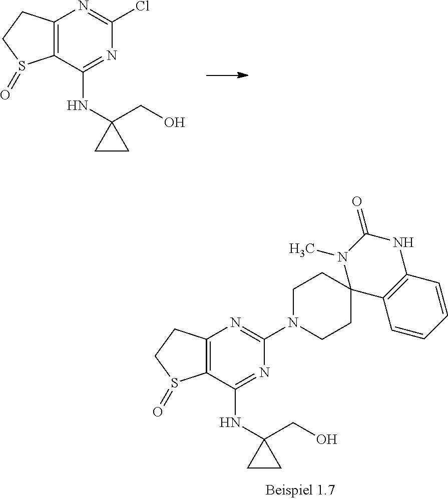 [SMILES: O=S1CCc2nc(Cl)nc(NC3(CO)CC3)c21 ; CN1C(=O)Nc2ccccc2C12CCN(c1nc3c(c(NC4(CO)CC4)n1)S(=O)CC3)CC2]

Starting from (IV-2) (see 2.4) and 3′-methyl-1′H-spiro[piperidin-4,4′-quinazolin]-2′(3′H)-one (Chem. Pharm. Bull. 1988, 4659) (0.1 mmol) are combined with 400 μl NMP and heated to 120° C. for 30 min in the microwave. Then 600 μL DMF are added, the reaction solution is purified by preparative HPLC-MS (method A) and the product fractions are freeze-dried. Analytical HPLC-MS (method C): RT=1.52 min.

### 8. Synthesis of {1-[2-(4-benzo[d]isoxazol-3-yl-piperidin-1-yl)-5-oxo-6,7-dihydro-5H-5λ⁴-thieno[3,2-d]pyrimidin-4-ylamino]-cyclopropyl}-methanol (Example 1.8)

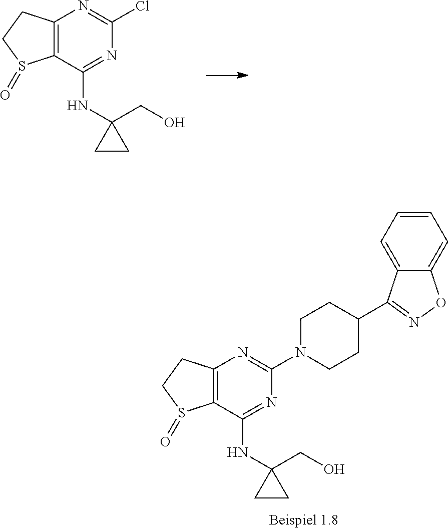 [SMILES: O=S1CCc2nc(Cl)nc(NC3(CO)CC3)c21 ; O=S1CCc2nc(N3CCC(c4noc5ccccc45)CC3)nc(NC3(CO)CC3)c21]

Starting from (IV-2) (see 2.4) and 3-piperidin-4-yl-benzo[d]isoxazole Example 1.8 may be prepared and purified analogously to Example 1.7 (see 7.). Analytical HPLC-MS (method C): RT=1.7 min.

### 9. Synthesis of (1-{2-[4-(2-ethyl-5-FLUORO-1H-indol-3-yl)-piperidin-1-yl]-5-oxo-6,7-dihydro-5H-5λ⁴-thieno[3,2-d]pyrimidin-4-ylamino}-cyclopropyl)-methanol (Example 1.9)

### 9.1 2-but-1-ynyl-4-fluorophenylamine

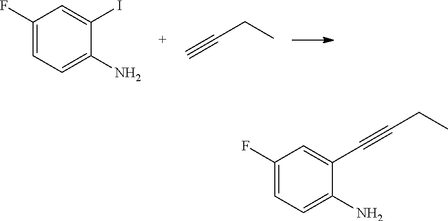

80 ml of tetrahydrofuran is placed under argon. 5 g of 4-fluoro-2-ioodphenylamine, 0.74 g dichlorobis(triphenylphosphine) palladium(II), 0.2 g copper iodide and 8.8 ml triethylamine are added. 4 g of gaseous 1-butyne are passed through the suspension. The reaction mixture is stirred for 15 hours at ambient temperature under argon, then filtered through Celite and evaporated to dryness. 3.4 g product are obtained as a solid. ¹H NMR (400 MHz, DMSO): 2.45 (2H, q); 1.18 (3H, t).

### 9.2 2-ethyl-5-fluoro-1H-indole

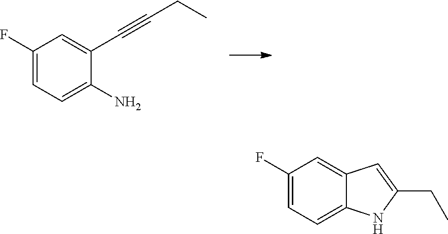

Under argon 4.9 g potassium-tert-butoxide are suspended in 25 ml N-methyl-2-pyrrolidinone and a suspension of 3.4 g 2-but-1-ynyl-4-fluorophenylamine in 25 ml N-methyl-2-pyrrolidinone is added dropwise. The reaction mixture is stirred for 3 hours at ambient temperature and mixed with water. The product is extracted with diethyl ether and purified by chromatography (silica gel, cyclohexane/ethyl acetate 100/0-90/10). 2.83 g product are obtained as a solid. ¹H NMR (400 MHz, DMSO): 2.72 (2H, q); 1.27 (3H, t).

### 9.3 2-ethyl-5-fluoro-3-(1,2,3,6-tetrahydropyridin-4-yl)-1H-indole

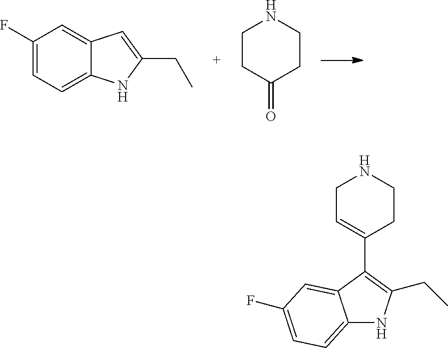 [SMILES: CCc1cc2cc(F)ccc2[nH]1 ; O=C1CCNCC1 ; CCc1[nH]c2ccc(F)cc2c1C1=CCNCC1]

2.83 g 2-ethyl-5-fluoro-1H-indole are suspended in 50 ml acetic acid and heated to 90° C. A suspension of 6.66 g of 4-piperidone in 15 ml phosphoric acid 2N is added. The reaction mixture is stirred for 3 hours at 90° C., combined with sodium hydroxide solution and the product is extracted with ethyl acetate. 2.85 g product are obtained as a solid. ¹H NMR (400 MHz, DMSO): 5.63 (1H, s); 2.73 (2H, q); 1.23 (3H, t).

### 9.4 2-ethyl-5-fluoro-3-piperidin-4-yl-1H-indole (V-1)

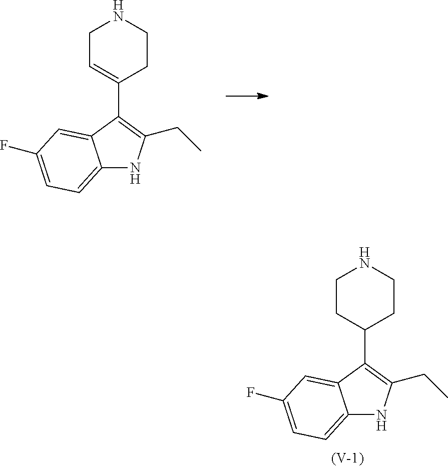

2.83 g 2-ethyl-5-fluoro-3-(1,2,3,6-tetrahydropyridin-4-yl)-1H-indole are suspended in 50 ml of methanol and hydrogenated with 0.3 g Pd/C 10% at normal pressure and ambient temperature. The catalyst is suction filtered and the filtrate is evaporated to dryness. 2.3 g (V-1) are obtained as a solid. ¹H NMR (400 MHz, DMSO): 2.70 (2H, q); 1.19 (3H, t).

### 9.5 (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol (Example 1.9)

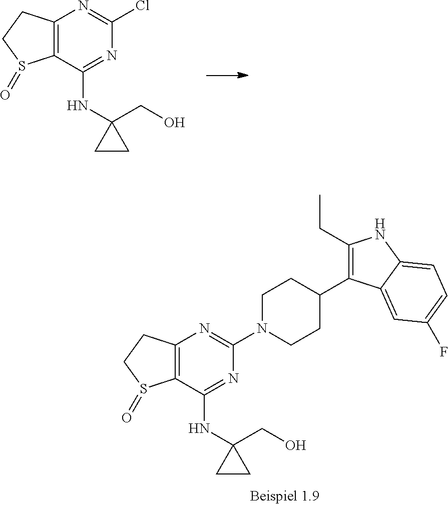 [SMILES: O=S1CCc2nc(Cl)nc(NC3(CO)CC3)c21 ; CCc1[nH]c2ccc(F)cc2c1C1CCN(c2nc3c(c(NC4(CO)CC4)n2)S(=O)CC3)CC1]

Starting from (IV-2) (see 2.4) and (V-1) Example 1.9 may be prepared and purified analogously to Example 1.7 (see 7.). Analytical HPLC-MS (method C): RT=1.78 min.

### 10. Synthesis of 1-[4-((S)-1-METHYL-6-oxopiperidin-3-ylamino)-5-oxo-6,7-dihydro-5H-5λ⁴-thieno[3,2-d]pyrimidin-2-yl]-4-phenylpiperidin-4-carbonitrile (Example 1.10)

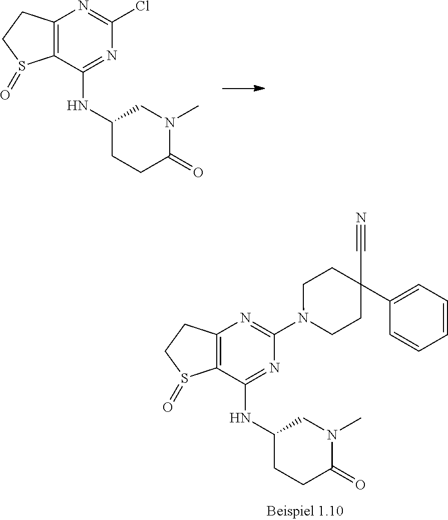 [SMILES: CN1C[C@@H](Nc2nc(Cl)nc3c2S(=O)CC3)CCC1=O ; CN1C[C@@H](Nc2nc(N3CCC(C#N)(c4ccccc4)CC3)nc3c2S(=O)CC3)CCC1=O]

Starting from (IV-5) (see 5.5) and 4-phenylpiperidin-4-carbonitrile Example 1.10 may be prepared and purified analogously to Example 1.7 (see 7). Analytical HPLC-MS (method C): RT=1.71 min.

### 11. Synthesis of 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(3H)-one (Example 1.11)

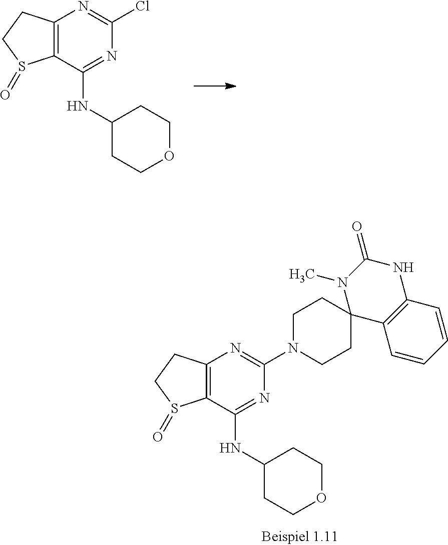 [SMILES: O=S1CCc2nc(Cl)nc(NC3CCOCC3)c21 ; CN1C(=O)Nc2ccccc2C12CCN(c1nc3c(c(NC4CCOCC4)n1)S(=O)CC3)CC2]

Starting from (IV-6) (see 6.2) and 3′-methyl-1′H-spiro[piperidin-4,4′-quinazolin]-2′(3H)-one (Chem. Pharm. Bull. 1988, 4659) Example 1.11 may be prepared and purified analogously to Example 1.7 (see 7.). Analytical HPLC-MS (method C): RT=1.56 min.

### 12. Synthesis of (3-fluorophenyl)[5-oxo-2-(3,4,5,6-tetrahydro-2H-[4,4′]bipyridinyl-1-yl)-6,7-dihydro-5H-5λ⁴-thieno[3,2-d]pyrimidin-4-yl]-amine (Example 1.12)

### 12.1 (2-chloro-6,7-dihydrothieno[3,2-d]pyrimidin-4-yl)-(3-fluorophenyl)-amine (III-7)

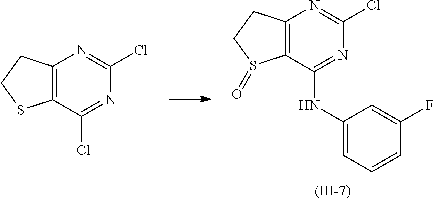 [SMILES: Clc1nc(Cl)c2c(n1)CCS2 ; O=S1CCc2nc(Cl)nc(Nc3cccc(F)c3)c21 ; "(III-7)"]

4 g (II) are placed in 15 ml dimethylformamide, then 4.5 ml diisopropylethylamine and then 2.5 ml 3-fluorophenylamine are added. The reaction mixture is heated to 120° C. until there is no further reaction, and after cooling evaporated down. The residue is mixed with water. The product is extracted with dichloromethane and purified by chromatography (silica gel, petroleum ether/ethyl acetate 80/20 to 60/40). 2.6 g (III-7) are obtained as a solid. Analytical HPLC (method A): RT=3.27 min

### 12.2 2-chloro-5-oxo-6,7-dihydro-5H-5λ⁴-thieno[3,2-d]pyrimidin-4-yl)-(3-fluorophenyl)-amine (IV-7)

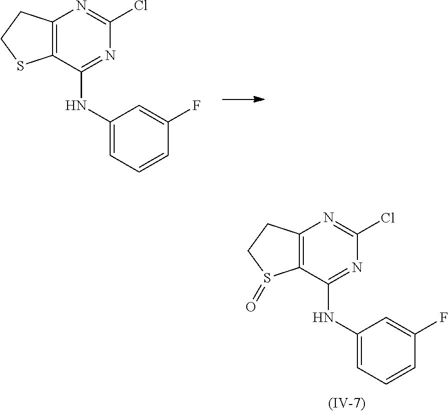 [SMILES: Fc1cccc(Nc2nc(Cl)nc3c2SCC3)c1 ; O=S1CCc2nc(Cl)nc(Nc3cccc(F)c3)c21 ; "(IV-7)"]

0.102 g S-(−)-1,1′-bi-2-naphthol are placed in 0.5 ml chloroform under argon, then 0.052 ml titanium(IV)isopropoxide and 0.064 ml of water are added. The reaction mixture is stirred for 45 minutes at ambient temperature. Then a suspension of 0.5 g (III-7) in 25 ml chloroform is added. The reaction mixture is cooled to −2°/−4° C. and after 20 minutes 0.323 ml tert-butylhydroperoxide 5-6 M in decane are added dropwise. The reaction mixture is stirred further at −2/−4° C. until there is no further reaction, and mixed with water. The product is extracted with dichloromethane and purified by chromatography (silica gel, dichloromethane/methanol 100/0 to 95/5). 0.47 g (IV-7) are obtained as a solid. Analytical HPLC-MS (method A): RT=1.15 min.

### 12.3 (3-fluorophenyl)-[5-oxo-2-(3,4,5,6-tetrahydro-2H-[4,4′]bipyridinyl-1-yl)-6,7-dihydro-5H-5λ⁴-thieno[3,2-d]pyrimidin-4-yl]-amine (Example 1.12)

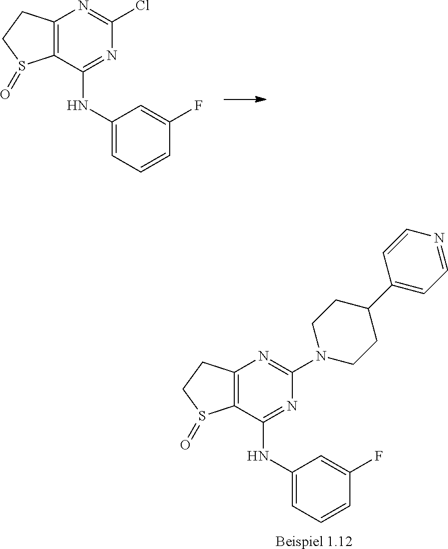

Starting from (IV-7) (see 12.2) and 1,2,3,4,5,6-hexahydro-[4,4]bipyridinyl Example 1.12 may be prepared and purified as the trifluoroacetate analogously to Example 1.7 (see 7.). Analytical HPLC-MS (method C): RT=1.55 min.

### 13. Synthesis of {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine (Example 1.13)

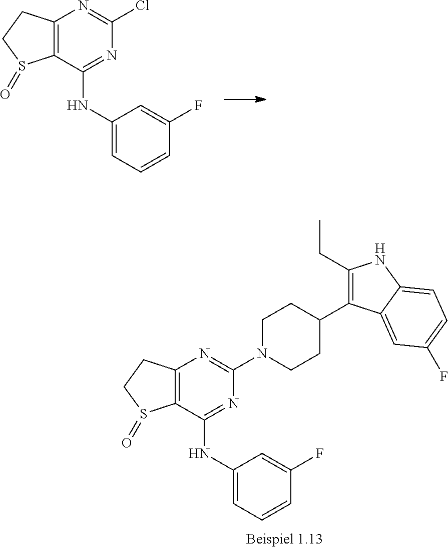

Starting from (IV-7) (see 12.2) and (V-1) (see 9.4) Example 1.13 may be prepared and purified analogously to Example 1.7 (see 7.). Analytical HPLC-MS (method C): RT=2.12 min.

### 14. Synthesis of (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol (Example 1.14)

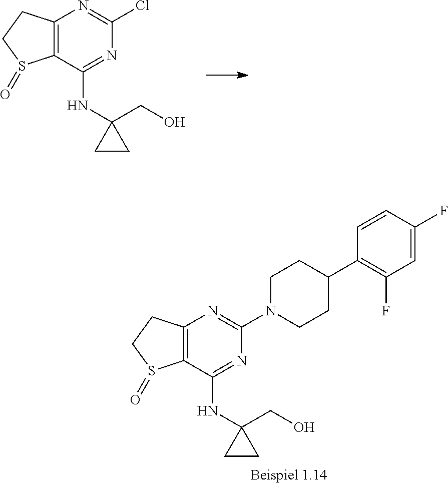

Starting from (IV-2) (see 2.4) and 4-(2,4-difluorophenyl)-piperidine Example 1.14 may be prepared analogously to Example 1.7 (see 7.). The product may be purified by chromatography (preparative HPLC, method B). Analytical HPLC-MS (method D): RT=1.18 min.

### 15. Synthesis of {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine (Example 1.15)

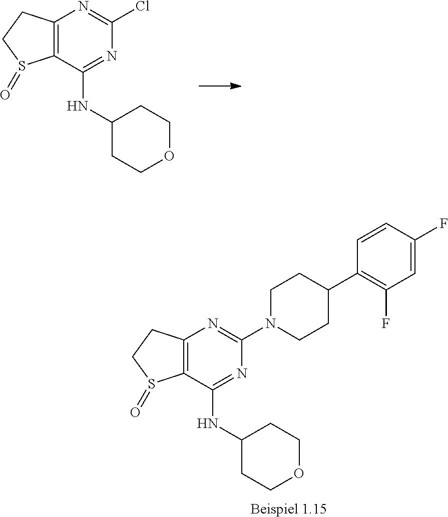

Starting from (IV-6) (see 6.2) and 4-(2,4-difluorophenyl)-piperidine Example 1.15 may be prepared and purified analogously to Example 1.14 (see 14.). Analytical HPLC-MS (method D): RT=1.23 min.

### 16. Synthesis of (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one (Example 1.16)

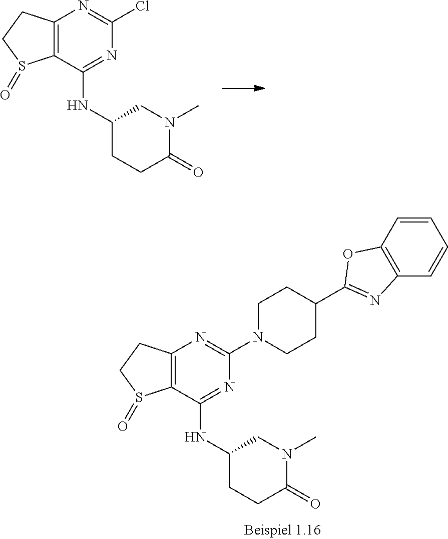 [SMILES: CN1C[C@@H](Nc2nc(Cl)nc3c2S(=O)CC3)CCC1=O ; CN1C[C@@H](Nc2nc(N3CCC(c4nc5ccccc5o4)CC3)nc3c2S(=O)CC3)CCC1=O]

Starting from (IV-5) (see 5.5) and 2-piperidin-4-yl-benzoxazole Example 1.16 may be prepared and purified analogously to Example 1.14 (see 14.). Analytical HPLC-MS (method B): RT=1.18 min.

### 17. Synthesis of (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol (Example 1.17)

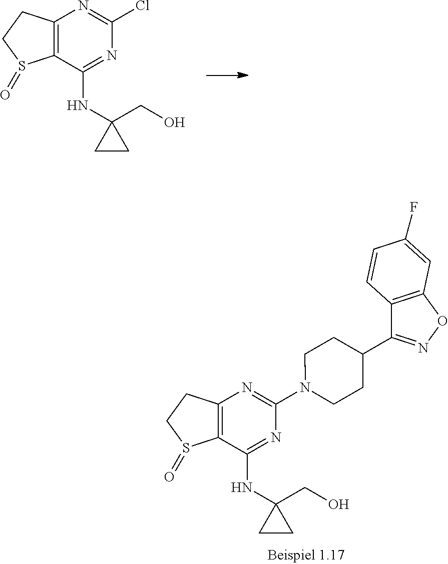 [SMILES: O=S1CCc2nc(Cl)nc(NC3(CO)CC3)c21 ; O=S1CCc2nc(N3CCC(c4noc5cc(F)ccc45)CC3)nc(NC3(CO)CC3)c21]

Starting from (IV-2) (see 2.4) and 6-fluoro-3-piperidin-4-yl-benzo[d]isoxazole Example 1.17 may be prepared and purified analogously to Example 1.7 (see 7.). Analytical HPLC-MS (method C): RT=1.76 min.

### 18. Synthesis of (1-{2-[4-(5-fluorobenzo[d]isoxazol-3-yl)-piperidin-1-yl]-5-oxo-6,7-dihydro-5H-5λ⁴-thieno[3,2-]pyrimidin-4-ylamino}-cyclopropyl)-methanol (Example 1.18)

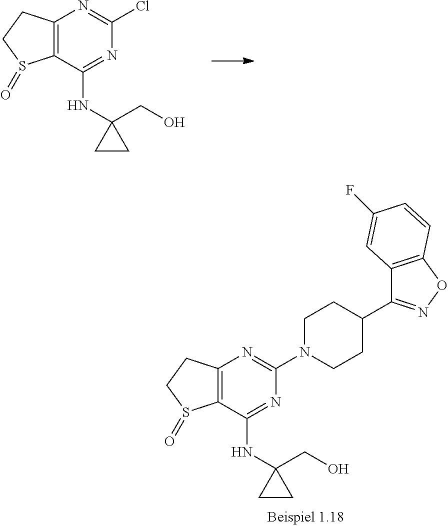 [SMILES: O=S1CCc2nc(Cl)nc(NC3(CO)CC3)c21 ; O=S1CCc2nc(N3CCC(c4noc5ccc(F)cc45)CC3)nc(NC3(CO)CC3)c21]

Starting from (IV-2) (see 2.4) and 5-fluoro-3-piperidin-4-yl-benzo[d]isoxazole Example 1.17 may be prepared and purified analogously to Example 1.7 (see 7.). Analytical HPLC-MS (method C): RT=1.74 min.

### 19. Synthesis of {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine (Example 1.19)

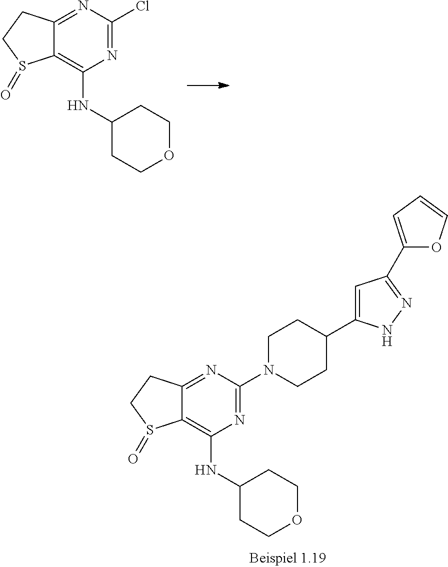 [SMILES: O=S1CCc2nc(Cl)nc(NC3CCOCC3)c21 ; O=S1CCc2nc(N3CCC(c4cc(-c5ccco5)n[nH]4)CC3)nc(NC3CCOCC3)c21]

Starting from (IV-6) (see 6.2) and 4-(5-furan-2-yl-2H-pyrazol-3-yl)-piperidine Example 1.19 may be prepared and purified analogously to Example 1.11 (see 11.). Analytical HPLC-MS (method C): RT=1.64 min.

### 20. Synthesis of (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine (Example 1.20)

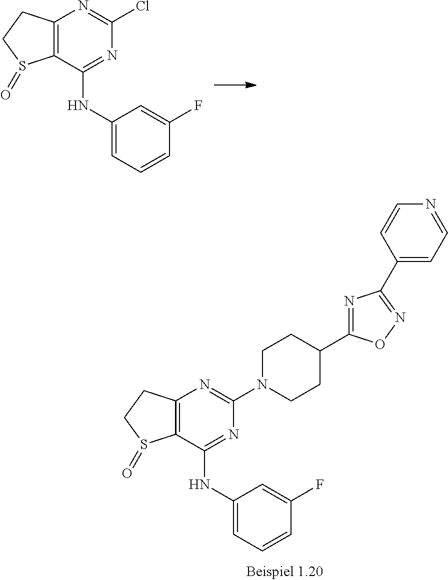 [SMILES: O=S1CCc2nc(Cl)nc(Nc3cccc(F)c3)c21 ; O=S1CCc2nc(N3CCC(c4nc(-c5ccncc5)no4)CC3)nc(Nc3cccc(F)c3)c21]

Starting from (IV-7) (see 12.2) and 4-(5-piperidin-4-yl-[1,2,4]oxadiazol-3-yl)-pyridine Example 1.20 may be prepared and purified as the trifluoroacetate analogously to Example 1.7 (see 7.). Analytical HPLC-MS (method C): RT=1.72 min.

### 21. Synthesis of (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol (Example 1.21)

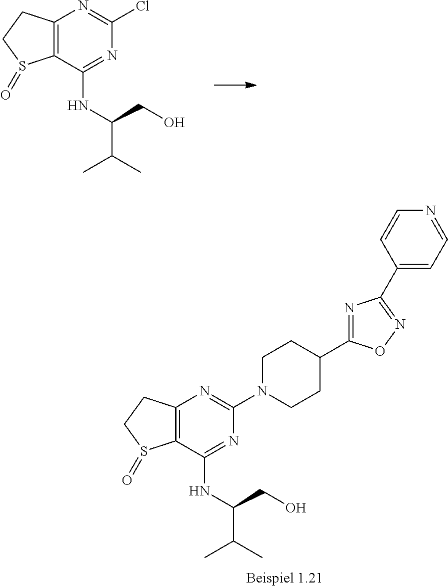 [SMILES: CC(C)[C@H](CO)Nc1nc(Cl)nc2c1S(=O)CC2 ; CC(C)[C@H](CO)Nc1nc(N2CCC(c3nc(-c4ccncc4)no3)CC2)nc2c1S(=O)CC2]

Starting from (IV-1) (see 1.2) and 4-(5-piperidin-4-yl-[1,2,4]oxadiazol-3-yl)-pyridine Example 1.21 may be prepared and purified as the trifluoroacetate analogously to Example 1.7 (see 7.). Analytical HPLC-MS (method C): RT=1.48 min.

### 22. Synthesis of: (S)-5-{2-[4-(4-fluorophenoxy)-piperidin-1-yl]-5-oxo-6,7-dihydro-5H-5λ⁴-thieno[3,2-d]pyrimidin-4-ylamino}-1-methylpiperidin-2-one (Example 1.22)

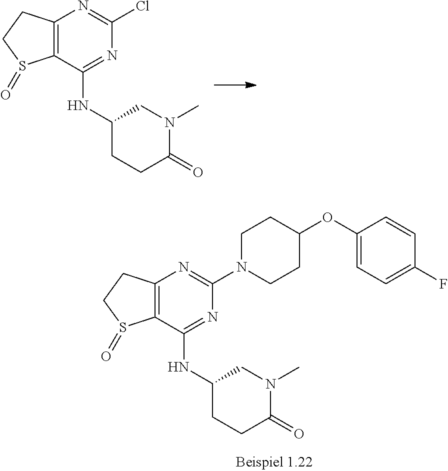 [SMILES: CN1C[C@@H](Nc2nc(Cl)nc3c2S(=O)CC3)CCC1=O ; CN1C[C@@H](Nc2nc(N3CCC(Oc4ccc(F)cc4)CC3)nc3c2S(=O)CC3)CCC1=O]

Starting from (IV-5) (see 5.5) and 4-(4-fluorophenoxy)-piperidine Example 1.22 may be prepared and purified analogously to Example 1.16 (see 16.). Analytical HPLC-MS (method A): RT=1.15 min.

### 23. Synthesis of: (2-{4-[4-(4,5-dihydroxazol-2-yl)-phenoxy]-piperidin-1-yl}-5-oxo-6,7-dihydro-5H-5λ⁴-thieno[3,2-d]pyrimidin-4-yl)-(tetrahydro Pyran-4-yl)-amine (Example 1.23)

### 23.1 tert.butyl 4-(toluene-4-sulphonyloxy)-piperidine-1-carboxylate

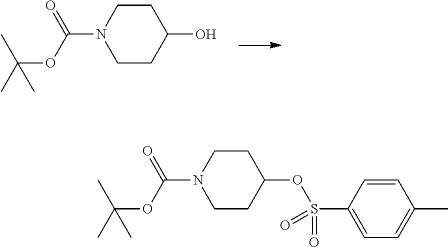 [SMILES: CC(C)(C)OC(=O)N1CCC(O)CC1 ; Cc1ccc(S(=O)(=O)OC2CCN(C(=O)OC(C)(C)C)CC2)cc1]

5 g tert.butyl 4-hydroxypiperidine-1-carboxylate are placed in 15 ml of pyridine, then 4.7 g p-toluenesulphonyl chloride are added batchwise. The reaction mixture is stirred at ambient temperature, after 12 hours poured onto ice water and the mixture obtained is stirred for a further hour at ambient temperature. The precipitated solid is suction filtered and dried. 7.5 g product are obtained.

### 23.2 tert.butyl 4-[4-(4,5-dihydroxazol-2-yl)-phenoxyl]-piperidine-1-carboxylate

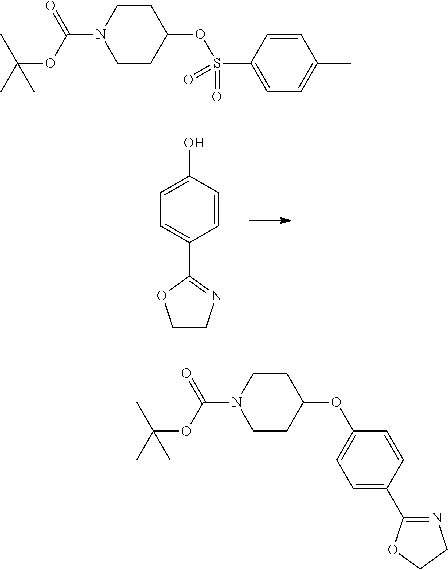

2.0 g 4-(4,5-dihydroxazol-2-yl)-phenol (see U.S. Pat. No. 5,491,201) are placed in 30 ml dimethylformamide, then 3.3 g potassium carbonate and 4.2 g tert.butyl 4-(toluene-4-sulphonyloxy)-piperidine-1-carboxylate are added. The reaction mixture is stirred at 75° C., after 12 hours it is mixed with water and the precipitated solid is suction filtered and dried. 2.8 g product are obtained.

### 23.3 4-[4-(4,5-dihydroxazol-2-yl)-phenoxy]-piperidine (V-2)

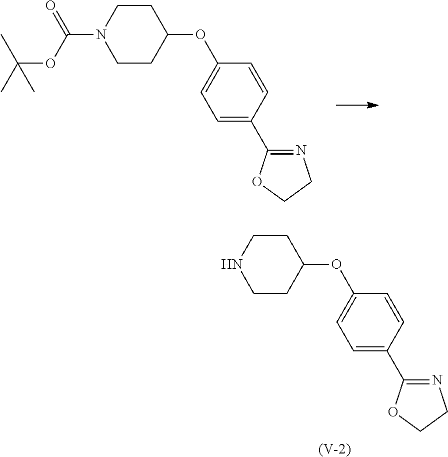

50 mg tert.butyl 4-[4-(4,5-dihydroxazol-2-yl)-phenoxyl]-piperidine-1-carboxylate are taken and combined with 6 ml of a (5/1) dichloromethane/trifluoroacetic acid mixture. The reaction mixture is stirred at ambient temperature and after 15 min a saturated NaHCO₃ solution is carefully added. The organic phase is dried and evaporated to dryness. 20 mg (V-2) are obtained.

### 23.4 (2-{4-[4-(4,5-dihydroxazol-2-yl)-phenoxyl]-piperidin-1-yl}-5-oxo-6,7-dihydro-5H-5λ⁴-thieno[3,2-d]pyrimidin-4-yl)-(tetrahydropyran-4-yl)-amine (Example 1.23)

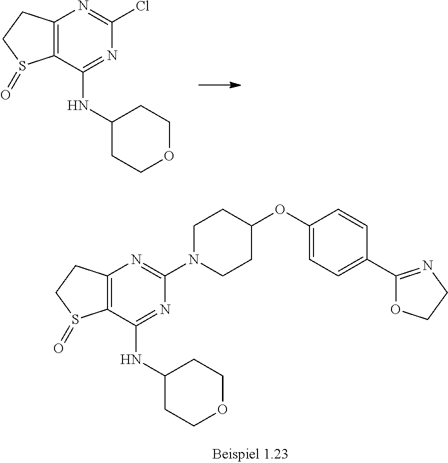 [SMILES: O=S1CCc2nc(Cl)nc(NC3CCOCC3)c21 ; O=S1CCc2nc(N3CCC(Oc4ccc(C5=NCCO5)cc4)CC3)nc(NC3CCOCC3)c21]

Starting from (IV-6) (see 6.2) and (V-2) Example 1.23 may be prepared and purified analogously to Example 1.14 (see 14.). Analytical HPLC-MS (method A): RT=0.99 min.

### 24. Synthesis of: 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid (Example 1.24)

### 24.1 methyl 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoate

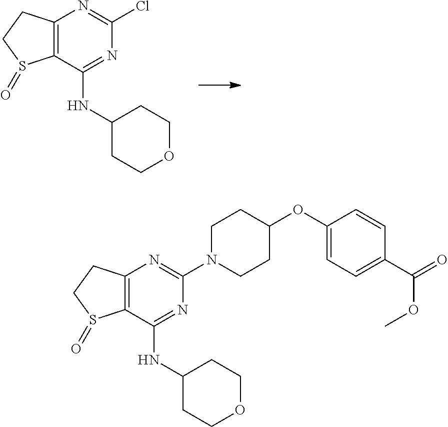 [SMILES: O=S1CCc2nc(Cl)nc(NC3CCOCC3)c21 ; COC(=O)c1ccc(OC2CCN(c3nc4c(c(NC5CCOCC5)n3)S(=O)CC4)CC2)cc1]

Starting from (IV-6) (see 6.2) and methyl 4-(piperidin-4-yloxy)-benzoate (J. Med. Chem. 2002, 3406) methyl 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoate may be prepared and purified analogously to Example 1.15 (see 15.). Analytical HPLC-MS (method A): RT=1.17 min.

### 24.2 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid (Example 1.24)

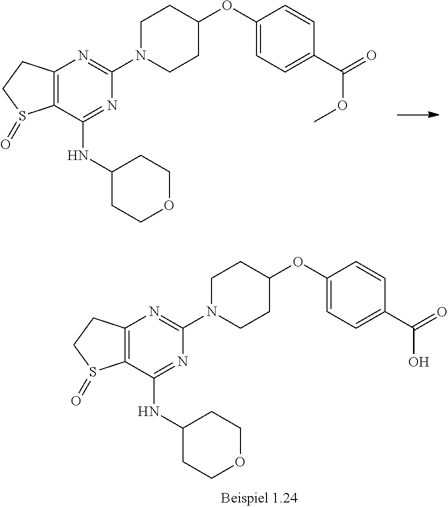 [SMILES: COC(=O)c1ccc(OC2CCN(c3nc4c(c(NC5CCOCC5)n3)S(=O)CC4)CC2)cc1 ; O=C(O)c1ccc(OC2CCN(c3nc4c(c(NC5CCOCC5)n3)S(=O)CC4)CC2)cc1]

80 mg of methyl 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoate are placed in 1.5 ml of methanol, then 560 μl of a 1N NaOH solution are added. The reaction mixture is stirred at 50° C. until there is no further reaction, then combined with a 1 M HCl solution. The product is extracted with dichloromethane. 77 mg Example 1.24 are obtained as a solid. Analytical HPLC-MS (method B): RT=1.19 min.

### 25. Synthesis of 2-(1-{2-[4-(4-chlorophenyl)-piperidin-1-yl]-5-oxo-6,7-dihydro-5H-5λ⁴-thieno[3,2-d]pyridimin-4-ylamino}-cyclopropyl)-propan-2-ol (Example 1.25)

### 25.1 2-[1-(2-chloro-6,7-dihydrothieno[3,2-d]pyrimidin-4-ylamino)-cyclopropyl]-propan-2-ol (III-8)

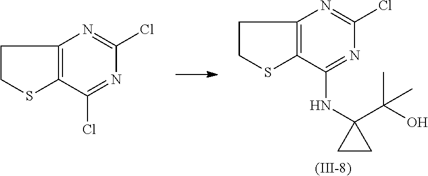 [SMILES: Clc1nc(Cl)c2c(n1)CCS2 ; CC(C)(O)C1(Nc2nc(Cl)nc3c2SCC3)CC1 ; "(III-8)"]

2.7 g (II) are placed in 30 ml dioxane, then 6.8 ml diisopropyl-ethylamine and 1.8 g 2-(1-aminocyclopropyl)-propan-2-ol (see Liebigs Ann. Chem. 1978, 1194) are added. The reaction mixture is heated to 160° C. until there is no further reaction, and after cooling it is evaporated to dryness. The residue is combined with ice water. The product is extracted with dichloromethane and purified by chromatography. 125 mg (III-8) are obtained as a solid. Analytical HPLC-MS (method A): RT=1.08 min.

### 25.2 2-[1-(2-chloro-5-oxo-6,7-dihydro-5H-5λ⁴-thieno[3,2-d]pyrimidin-4-ylamino)-cyclopropyl]-propan-2-ol (IV-8)

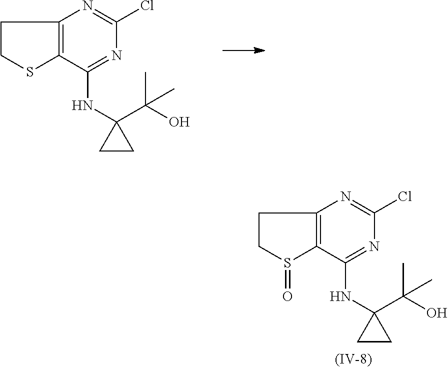

21.6 mg S-(−)-1,1′-bi-2-naphthol are placed in 1 ml chloroform under argon, then 11 μl titanium(IV)isopropoxide and 14 μl water are added. The reaction mixture is stirred for 1 hour at ambient temperature. Then a mixture of 120 mg (III-8) in 4 ml dichloromethane is added. The reaction mixture is cooled to −5° C. and after 30 minutes 69.5 μl tert-butylhydroperoxide 5-6 M in decane are added dropwise. The reaction mixture is stirred at −5° C. After 2 days the same amounts again of S-(−)-1,1′-bi-2-naphthol, titanium(IV)isopropoxide, water and tert-butylhydroperoxide are added. The reaction mixture is stirred at −5° C. to 5° C. until there is no further reaction, mixed with water and made basic with NH₄OH. The organic phase is evaporated to dryness and the product is purified by chromatography (preparative HPLC, method B). 105 mg (IV-8) are obtained. Analytical HPLC-MS (method A): RT=0.96 min.

### 25.3 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol (Example 1.25)

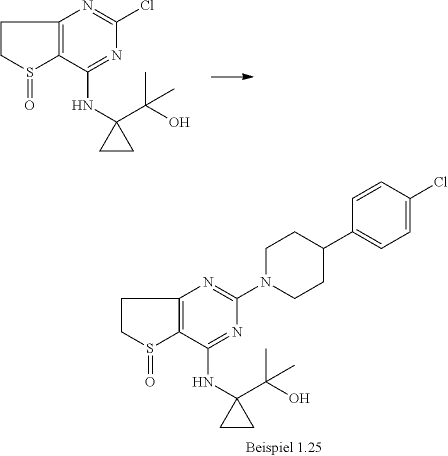 [SMILES: CC(C)(O)C1(Nc2nc(Cl)nc3c2S(=O)CC3)CC1 ; CC(C)(O)C1(Nc2nc(N3CCC(c4ccc(Cl)cc4)CC3)nc3c2S(=O)CC3)CC1]

Starting from (IV-8) and 4-(4-chlorophenyl)-piperidine hydrochloride Example 1.25 may be prepared and purified analogously to Example 1.14 (see 14.). Analytical HPLC-MS (method B): RT=1.37 min.

### 26. Synthesis of: {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-tetrahydro pyran-4-yl)-amine (Example 1.26)

### 26.1 tert.butyl 4-(1H-indol-3-yl)-piperidine-1-carboxylate

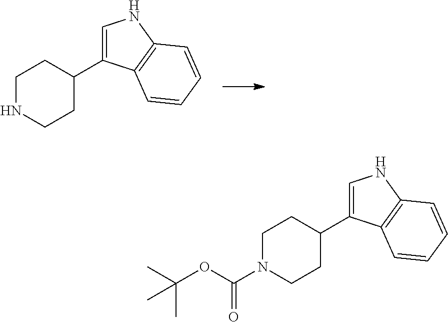 [SMILES: c1ccc2c(C3CCNCC3)c[nH]c2c1 ; CC(C)(C)OC(=O)N1CCC(c2c[nH]c3ccccc23)CC1]

10 g 3-piperidin-4-yl-1H-indole are placed in 300 mL THF and 10.9 g di-tert-butyl-dicarbonate are added. The reaction mixture is stirred overnight at ambient temperature and evaporated to dryness. The residue is mixed with water and the product is extracted with diethyl ether and purified by chromatography. 9 g of the product are obtained as a solid.

### 26.2 tert.butyl 4-(1-methyl-1H-indol-3-yl)-piperidine-1-carboxylate

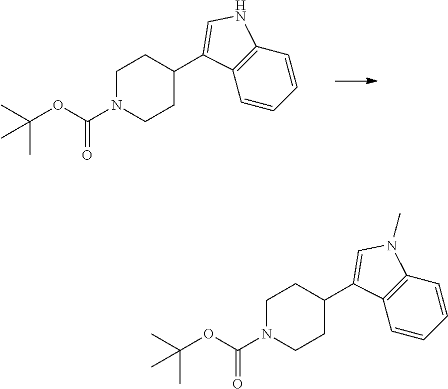 [SMILES: CC(C)(C)OC(=O)N1CCC(c2c[nH]c3ccccc23)CC1 ; Cn1cc(C2CCN(C(=O)OC(C)(C)C)CC2)c2ccccc21]

500 mg tert.butyl 4-(1H-indol-3-yl)-piperidine-1-carboxylate are placed in 8 ml dimethylformamide and 73.3 mg sodium hydride (60% in mineral oil) are added. After 15 min 175 μl methyl iodide are added. The reaction mixture is stirred at ambient temperature. After the reaction is complete the product is purified directly by preparative HPLC (method C). 302 mg of the product are obtained as an oil. Analytical HPLC-MS (method A): RT=1.65 min.

### 26.3 5-tert-butyl-1-methyl-3-piperidin-4-yl-1H-indole (V-3)

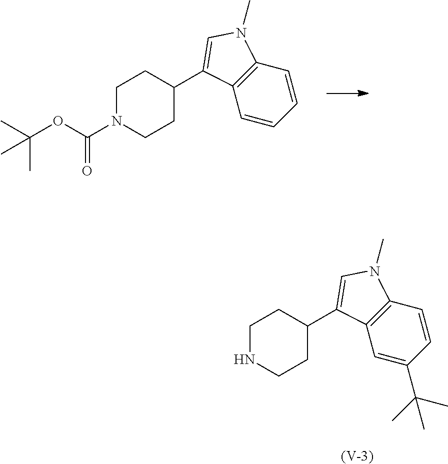 [SMILES: Cn1cc(C2CCN(C(=O)OC(C)(C)C)CC2)c2ccccc21 ; Cn1cc(C2CCNCC2)c2cc(C(C)(C)C)ccc21 ; "(V-3)"]

365 mg tert.butyl 4-(1-methyl-1H-indol-3-yl)-piperidine-1-carboxylate are placed in 1 ml dichloromethane and combined with 1.03 ml trifluoroacetic acid. The reaction mixture is stirred at ambient temperature. After 12 and 16 h another 1.03 ml of trifluoroacetic acid are added. After a further 12 h the reaction mixture is evaporated to dryness. The residue is combined with toluene and evaporated to dryness. The residue is triturated with diethyl ether, the precipitate is suction filtered and dried. 154 mg (V-3) are obtained as a solid. Analytical HPLC-MS (method A): RT=1.34 min.

### 26.4 {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine (Example 1.26)

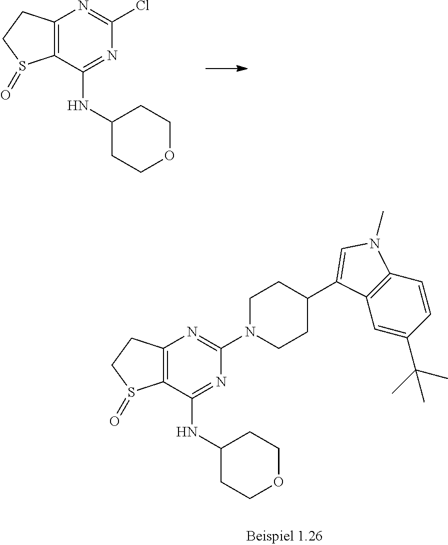 [SMILES: O=S1CCc2nc(Cl)nc(NC3CCOCC3)c21 ; Cn1cc(C2CCN(c3nc4c(c(NC5CCOCC5)n3)S(=O)CC4)CC2)c2cc(C(C)(C)C)ccc21]

Starting from (IV-6) (see 6.2) and (V-3) Example 1.26 may be prepared and purified analogously to Example 1.14 (see 14.). Analytical HPLC-MS (method B): RT=1.16 min.

### 27. Synthesis of: {2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine (Example 1.27)

### 27.1 tert.butyl 14-(5-furan-2-yl-2H-pyrazol-3-yl)-piperidine-1-carboxylate

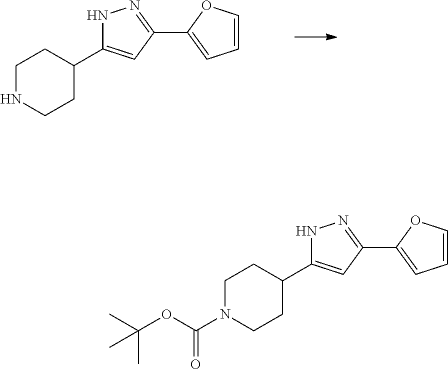 [SMILES: c1coc(-c2cc(C3CCNCC3)[nH]n2)c1 ; CC(C)(C)OC(=O)N1CCC(c2cc(-c3ccco3)n[nH]2)CC1]

200 mg 4-(5-furan-2-yl-2H-pyrazol-3-yl)-piperidine are placed in 2 ml dioxane. Then 0.34 ml of water and 155 mg sodium carbonate are added. The reaction mixture is stirred at ambient temperature. After 5 min 204 mg di-tert-butyl-dicarbonate are added. After 3 h the reaction mixture is mixed with water and the product is extracted with dichloromethane. 300 mg of product are obtained as an oil. Analytical HPLC-MS (method B): RT=1.54 min.

### 27.2 tert.butyl 4-(5-furan-2-yl-2-methyl-2H-pyrazol-3-yl)-piperidine-1-carboxylate and tert. butyl 4-(5-furan-2-yl-1-methyl-1H-pyrazol-3-yl)-piperidine-1-carboxylate

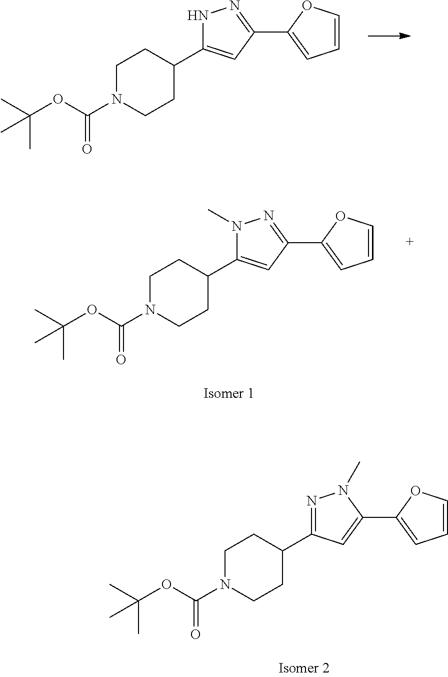 [SMILES: CC(C)(C)OC(=O)N1CCC(c2cc(-c3ccco3)n[nH]2)CC1 ; Cn1nc(-c2ccco2)cc1C1CCN(C(=O)OC(C)(C)C)CC1 ; Cn1nc(C2CCN(C(=O)OC(C)(C)C)CC2)cc1-c1ccco1]

250 mg tert.butyl 14-(5-furan-2-yl-2H-pyrazol-3-yl)-piperidine-1-carboxylate are placed in 1.5 ml dimethylformamide. The reaction mixture is cooled in the ice bath and 40 mg sodium hydride (60% in mineral oil) are added. After 10 min 60 μl methyl iodide are added. The reaction mixture is stirred for 30 min at 5° C. and then for 4 h at ambient temperature. The product is then purified directly by preparative HPLC (method D). 90 mg isomer 1 and 50 mg isomer 2 are obtained as a solid. Analytical HPLC-MS (method D): RT=1.33 min (isomer 1); RT=1.28 (isomer 2).

### 27.3 4-(5-furan-2-yl-1-methyl-1H-pyrazol-3-yl)-piperidine (V-4)

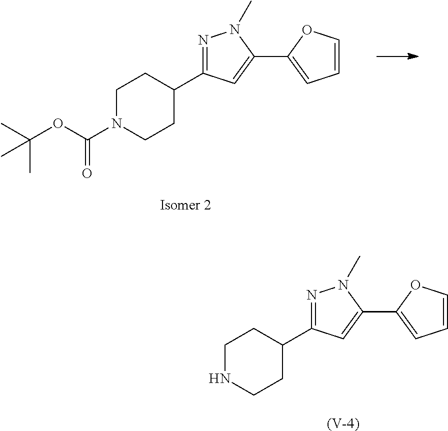 [SMILES: Cn1nc(C2CCN(C(=O)OC(C)(C)C)CC2)cc1-c1ccco1 ; Cn1nc(C2CCNCC2)cc1-c1ccco1 ; "(V-4)"]

47 mg isomer 2 are placed in 1 ml dichloromethane and 120 μl trifluoroacetic acid are added. The reaction mixture is stirred for 2 h at ambient temperature, then evaporated to dryness. The residue is combined with toluene and evaporated to dryness. The residue is mixed with water, made basic with conc. ammonia and the product is extracted with dichloromethane. 23 mg (V-4) are obtained as a solid. Analytical HPLC-MS (method B): RT=0.85 min

### 27. 4 {2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine (Example 1.27)

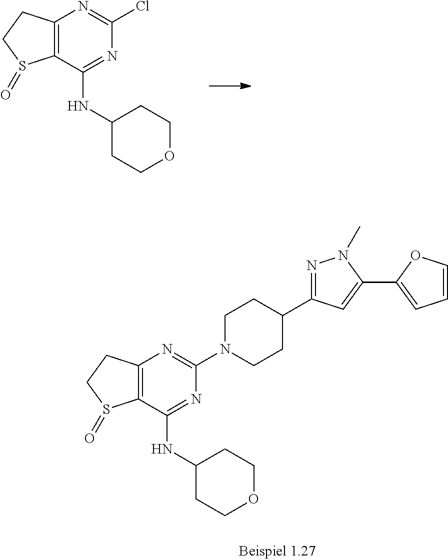 [SMILES: O=S1CCc2nc(Cl)nc(NC3CCOCC3)c21 ; Cn1nc(C2CCN(c3nc4c(c(NC5CCOCC5)n3)S(=O)CC4)CC2)cc1-c1ccco1]

Starting from (IV-6) (see 6.2) and (V-4) Example 1.27 may be prepared and purified analogously to Example 1.14 (see 14). Analytical HPLC-MS (method B): RT=1.21 min.

### 28. Synthesis of: (s)-5-(2-{4-[4-(4,5-dihydroxazol-2-yl)-phenoxy]-piperidin-1-yl}-5-oxo-6,7-dihydro-5H-5λ⁴-thieno[3,2-d]pyrimidin-4-ylamino)-1-methylpiperidin-2-one (Example 1.28)

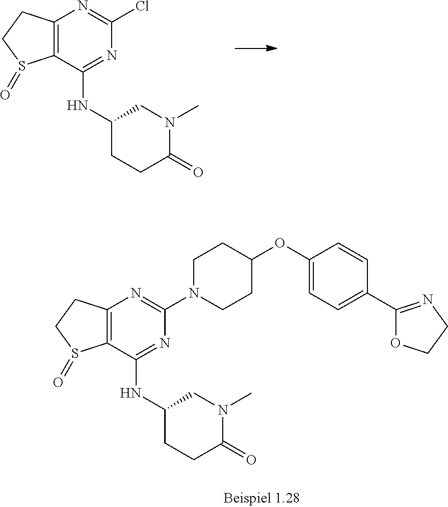 [SMILES: CN1C[C@@H](Nc2nc(Cl)nc3c2S(=O)CC3)CCC1=O ; CN1C[C@@H](Nc2nc(N3CCC(Oc4ccc(C5=NCCO5)cc4)CC3)nc3c2S(=O)CC3)CCC1=O]

Starting from (IV-5) (see 5.5) and (V-2) (see 23.3) Example 1.28 may be prepared and purified analogously to Example 1.16 (see 16.). Analytical HPLC-MS (method B): RT=1.07 min.

### 29. Synthesis of: {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine (Example 1.29)

### 29.1 4-(5-furan-2-yl-2-methyl-2H-pyrazol-3-yl)-piperidine (V-5)

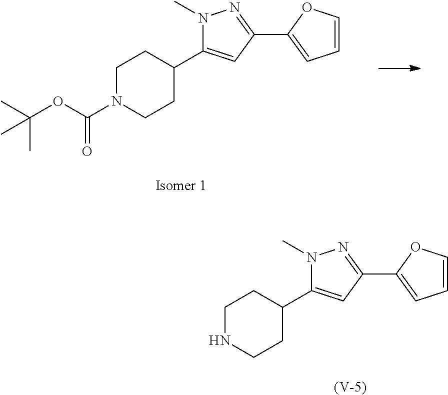 [SMILES: Cn1nc(-c2ccco2)cc1C1CCN(C(=O)OC(C)(C)C)CC1 ; Cn1nc(-c2ccco2)cc1C1CCNCC1 ; "(V-5)"]

Starting from isomer 1 (see 27.2), (V-5) may be prepared analogously to (V-4) (see 27.3). Analytical HPLC-MS (method D): RT=0.89 min.

### 29. 2 Synthesis of: {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine (Example 1.29)

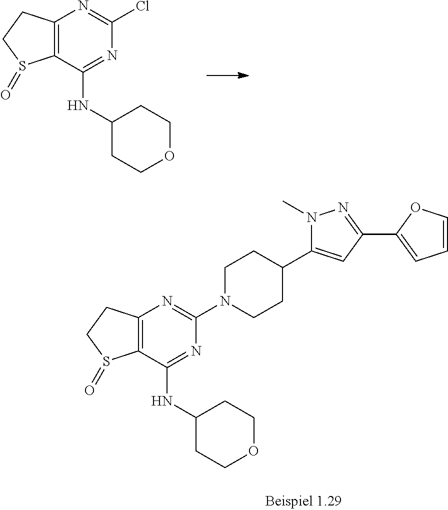 [SMILES: O=S1CCc2nc(Cl)nc(NC3CCOCC3)c21 ; Cn1nc(-c2ccco2)cc1C1CCN(c2nc3c(c(NC4CCOCC4)n2)S(=O)CC3)CC1]

Starting from (IV-6) (see 6.2) and (V-5) Example 1.29 may be prepared and purified analogously to Example 1.14 (see 14.). Analytical HPLC-MS (method B): RT=1.26 min.

### 30. Synthesis of: {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydro pyran-4-yl)-amine (Example 1.30)

### 30.1 methyl-(3-nitropyridin-4-yl)-amine

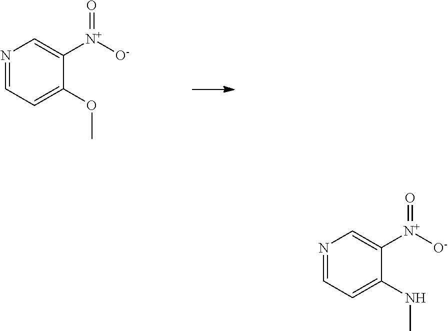

2.36 g 4-methoxy-3-nitro-pyridine and 2.33 ml methylamine (40% in water) are refluxed in 25 ml of ethanol for 3 h. Then the reaction mixture is evaporated to dryness. 2.3 g product are obtained as a solid.

### 30.2 N⁴-methylpyridin-3,4-diamine

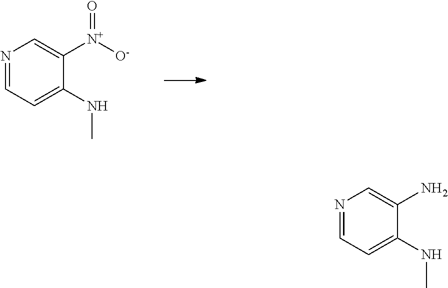

2.3 g methyl-(3-nitropyridin-4-yl)-amine are hydrogenated in 50 ml of methanol with 0.8 g Raney nickel for 2.5 h at 50° C. and 50 psi hydrogen pressure. The catalyst is filtered off and the filtrate is evaporated to dryness. The product is purified by chromatography (Alox, dichloromethane/methanol von 99/1 to 19/1). 1.55 g product are obtained as a solid. M.p: 163-165° C.

### 30.3 1-methyl-2-piperidin-4-yl-1H-imidazo[4,5-c]pyridine (V-6)

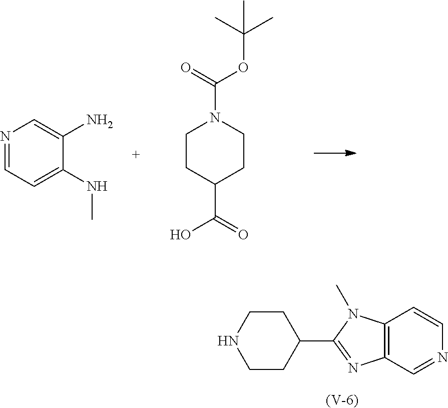

450 mg N⁴-methylpyridin-3,4-diamine and 838 mg mono-tert-butyl piperidine-1,4-dicarboxylate are heated to 200° C. in 8.6 g polyphosphoric acid for 4 h. After cooling the mixture is made basic with 4 N NaOH and acidified with trifluoroacetic acid. The mixture is purified by preparative HPLC (method C). 3.37 g (50%) (V-6) are obtained as the trifluoroacetate. Analytical HPLC-MS (method B): RT=0.30 min.

### 30.4 {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine (Example 1.30)

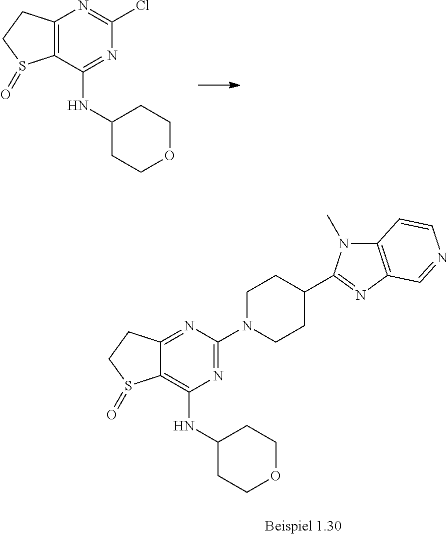 [SMILES: O=S1CCc2nc(Cl)nc(NC3CCOCC3)c21 ; Cn1c(C2CCN(c3nc4c(c(NC5CCOCC5)n3)S(=O)CC4)CC2)nc2cnccc21]

Starting from (IV-6) (see 6.2) and (V-6) Example 1.30 may be prepared and purified analogously to Example 1.14 (see 14). Analytical HPLC-MS (method D): RT=0.86 min.

### 31. Synthesis of 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide (Example 1.31)

### 31.1 tert.butyl 4-[(2-methoxyacetylamino)-methyl]-4-phenylpiperidine-1-carboxylate

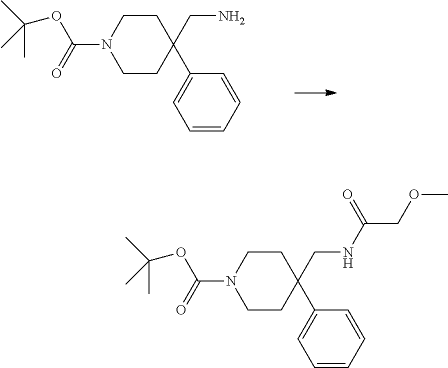 [SMILES: CC(C)(C)OC(=O)N1CCC(CN)(c2ccccc2)CC1 ; COCC(=O)NCC1(c2ccccc2)CCN(C(=O)OC(C)(C)C)CC1]

3.7 g of commercial tert.butyl 4-aminomethyl-4-phenyl-piperidine-1-carboxylate and 3 ml diisopropylethylamine are placed in 30 ml dichloromethane. Then 2.25 ml methoxyacetyl chloride are slowly added. The reaction mixture is stirred at ambient temperature until there is no further reaction, then mixed with water. The organic phase is evaporated to dryness. 4.7 g product are obtained as an oil.

### 31.2 2-methoxy-N-(4-phenylpiperidin-4-ylmethyl)-acetamide (V-7)

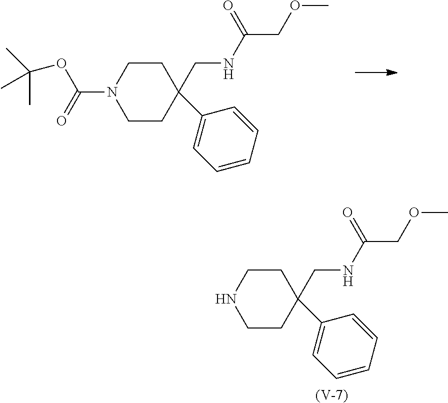

1 g of tert.butyl 4-[(2-methoxyacetylamino)-methyl]-4-phenylpiperidine-1-carboxylate is placed in 4 ml dichloromethane. Then 1.7 ml trifluoroacetic acid are added and the mixture is stirred overnight at ambient temperature. The reaction mixture is made basic with potassium carbonate and the organic phase is evaporated to dryness. 610 mg (V-7) are obtained as an oil.

### 31.3 Synthesis of 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide (Example 1.31)

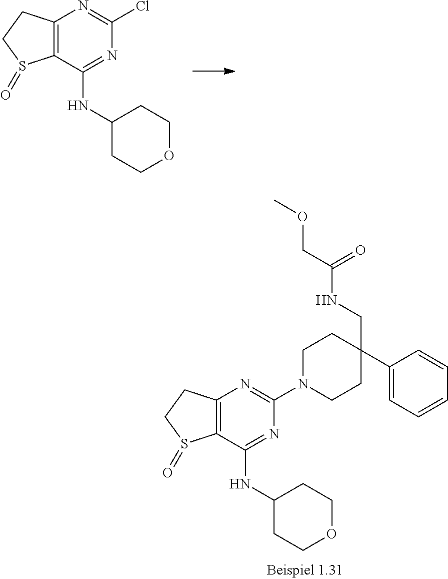 [SMILES: O=S1CCc2nc(Cl)nc(NC3CCOCC3)c21 ; COCC(=O)NCC1(c2ccccc2)CCN(c2nc3c(c(NC4CCOCC4)n2)S(=O)CC3)CC1]

Starting from (IV-6) (see 6.2) and (V-7) Example 1.31 may be prepared and purified analogously to Example 1.15 (see 15.). Analytical HPLC-MS (method B): RT=1.21 min.

### 32. Synthesis of: N-cyclopropyl-N-methyl-4-{1-[5-oxo-4-(tetrahydro pyran-4-ylamino)-6,7-dihydro-5H-5λ⁴-thieno[3,2-d]pyrimidin-2-yl]-piperidin-4-yl}-benzamide (Example 1.32)

### 32.1 tert.butyl 4-[4-(cyclopropylmethylcarbamoyl)-phenyl]-piperidine-1-carboxylate

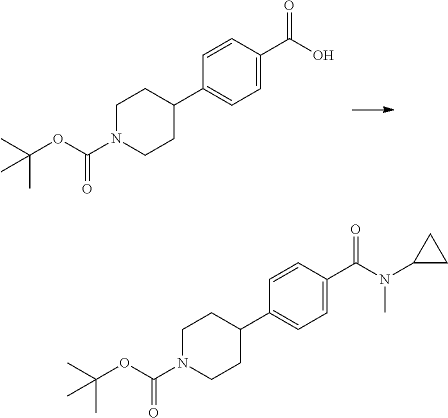 [SMILES: CC(C)(C)OC(=O)N1CCC(c2ccc(C(=O)O)cc2)CC1 ; CN(C(=O)c1ccc(C2CCN(C(=O)OC(C)(C)C)CC2)cc1)C1CC1]

500 mg tert.butyl 4-(4-carboxyphenyl)-piperidine-1-carboxylate are placed in 28 ml dimethylformamide, then 1.14 ml diisopropylethylamine and 747 mg HATU are added. The reaction mixture is stirred for 15 min at ambient temperature, then 194 mg cyclopropylmethylamine hydrochloride are added. The reaction mixture is stirred overnight at ambient temperature. Then the product is purified by preparative HPLC (method A). 480 mg product are obtained as an oil. Analytical HPLC-MS (method B): RT=1.64 min.

### 32.2 N-cyclopropyl-N-methyl-4-piperidin-4-yl-benzamide (V-8)

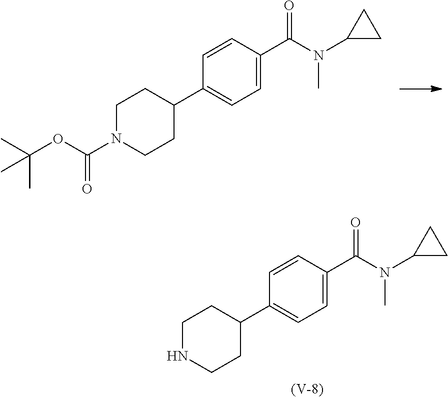 [SMILES: CN(C(=O)c1ccc(C2CCN(C(=O)OC(C)(C)C)CC2)cc1)C1CC1 ; CN(C(=O)c1ccc(C2CCNCC2)cc1)C1CC1 ; "(V-8)"]

480 mg tert.butyl 4-[4-(cyclopropylmethylcarbamoyl)-phenyl]-piperidine-1-carboxylate are placed in 7.8 ml dichloromethane and combined with 1.09 ml trifluoroacetic acid. The reaction mixture is stirred for 1.5 h at ambient temperature and then evaporated to dryness. The residue is combined with toluene and evaporated to dryness again. 444 mg (V-8) are obtained as the trifluoroacetate. Analytical HPLC-MS (method B): RT=1.11 min.

### 32.3 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide (Example 1.32)

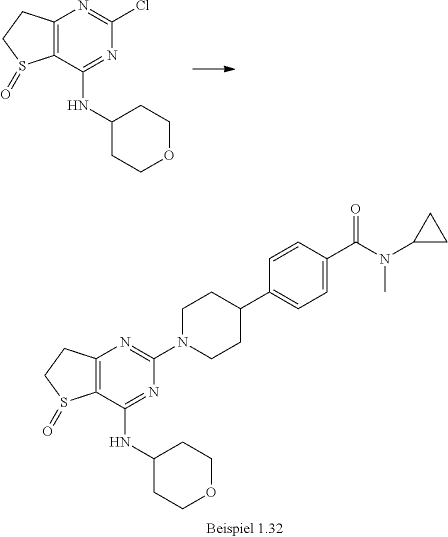 [SMILES: O=S1CCc2nc(Cl)nc(NC3CCOCC3)c21 ; CN(C(=O)c1ccc(C2CCN(c3nc4c(c(NC5CCOCC5)n3)S(=O)CC4)CC2)cc1)C1CC1]

Starting from (IV-6) (see 6.2) and (V-8) Example 1.32 may be prepared and purified analogously to Example 1.14 (see 14.). Analytical HPLC-MS (method D): RT=1.05 min.

### 33. Synthesis of: N-Cyclopropyl-N-Methyl-4-{1-[5-oxo-4-(tetrahydro-.pyran-4-ylamino)-6,7-dihydro-5H-5λ⁴-thieno[3,2-d]pyrimidin-2-yl]-piperidin-4-yloxy}-benzamide (Example 1.33)

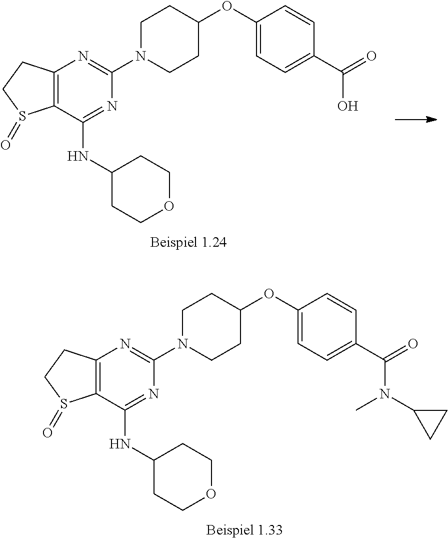 [SMILES: O=C(O)c1ccc(OC2CCN(c3nc4c(c(NC5CCOCC5)n3)S(=O)CC4)CC2)cc1 ; CN(C(=O)c1ccc(OC2CCN(c3nc4c(c(NC5CCOCC5)n3)S(=O)CC4)CC2)cc1)C1CC1]

55 mg of Example 1.24 (see 24.2) are placed in 2 ml dimethylformamide, then 81 μl diisopropylethylamine and 53.1 mg O-(7-azabenzotriazol-1-yl-)-N,N,N′,N′-tetramethyluronium hexafluorophosphate (HATU) are added. After 15 min 13.8 mg cyclopropylmethylamine hydrochloride are added. The reaction mixture is stirred at ambient temperature until there is no further reaction, and the product is directly purified by preparative HPLC (method B). 30 mg Example 1.33 are obtained as a solid. Analytical HPLC-MS (method D): RT=1.03 min.

### 34. Synthesis of: {5-oxo-2-[4-(pyridin-4-yloxy)-piperidin-1-yl]-6,7-dihydro-5H-5λ⁴-thieno[3,2-d]pyrimidin-4-yl}-(tetrahydropyran-4-yl)-amine (Example 1.34)

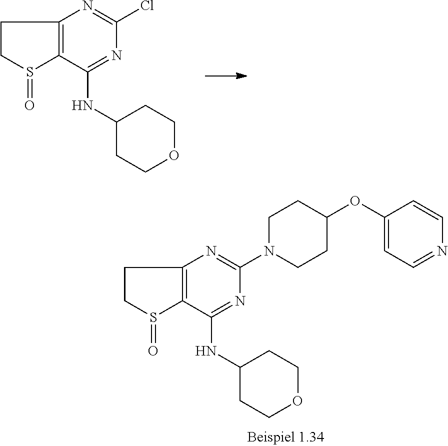 [SMILES: O=S1CCc2nc(Cl)nc(NC3CCOCC3)c21 ; O=S1CCc2nc(N3CCC(Oc4ccncc4)CC3)nc(NC3CCOCC3)c21]

Starting from (IV-6) (see 6.2) and 4-(piperidin-4-yloxy)-pyridine Example 1.34 may be prepared and purified analogously to Example 1.15 (see 15.). Analytical HPLC-MS (method B): RT=0.99 min.

### 35. Synthesis of: {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine (Example 1.35)

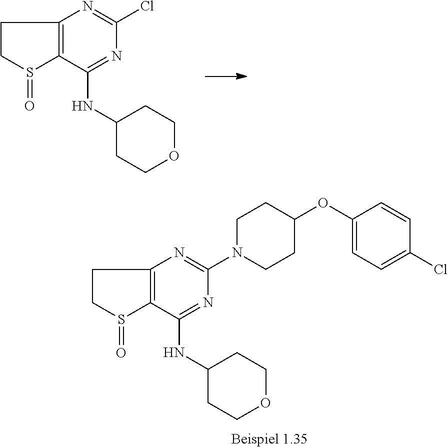 [SMILES: O=S1CCc2nc(Cl)nc(NC3CCOCC3)c21 ; O=S1CCc2nc(N3CCC(Oc4ccc(Cl)cc4)CC3)nc(NC3CCOCC3)c21]

Starting from (IV-6) (see 6.2) and 4-(4-chlorophenoxy)-piperidine Example 1.35 may be prepared and purified analogously to Example 1.15 (see 15.). Analytical HPLC-MS (method B): RT=1.39 min.

### 36. Synthesis of: (s)-1-methyl-5-{2-[4-(5-methyl-4-phenyl-oxazol-2-yl)-Piperidin-1-yl]-5-oxo-6,7-dihydro-5H-5λ⁴-thieno[3,2-d]pyrimidin-4-ylamino}-piperidin-2-one (Example 1.36)

### 36.1 4-(5-methyl-4-phenyloxazol-2-yl)-piperidine (V-9)

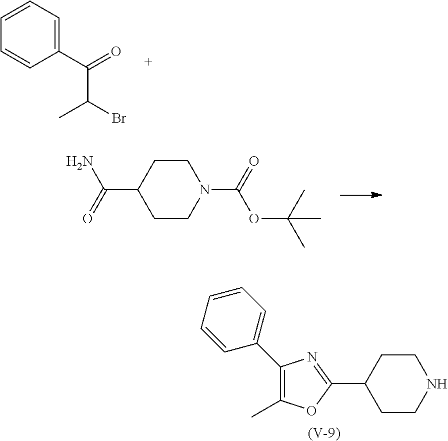

1.75 g 2-bromo-1-phenylpropan-1-one and 1.87 g tert.butyl 4-carbamoylpiperidine-1-carboxylate are placed in 0.5 ml NMP. The reaction mixture is heated to 160° C. for 20 min in the microwave and for 35 min in the oil bath, then after cooling it is taken up in methanol and evaporated to dryness. The residue is mixed with water, treated in the ultrasound bath and the insoluble oil is suction filtered. The mother liquor is purified by preparative HPLC (method C). 160 mg (V-9) are obtained as the trifluoroacetate. Analytical HPLC-MS (method B): RT=1.24 min.

### 36.2 (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one (Example 1.36)

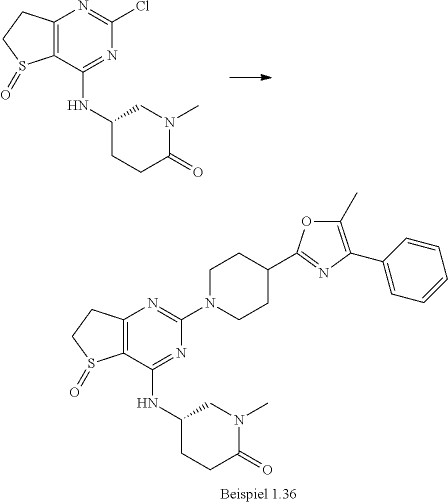 [SMILES: CN1C[C@@H](Nc2nc(Cl)nc3c2S(=O)CC3)CCC1=O ; Cc1oc(C2CCN(c3nc4c(c(N[C@H]5CCC(=O)N(C)C5)n3)S(=O)CC4)CC2)nc1-c1ccccc1]

Starting from (IV-5) (see 5.5) and (V-9) Example 1.36 may be prepared and purified analogously to Example 1.14 (see 14.). Analytical HPLC-MS (method D): RT=1.08 min.

### 37 Synthesis of: (1-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-cyclopropyl)-methanol (Example 1.37)

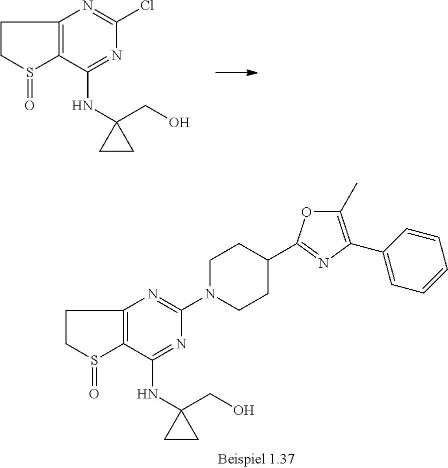 [SMILES: O=S1CCc2nc(Cl)nc(NC3(CO)CC3)c21 ; Cc1oc(C2CCN(c3nc4c(c(NC5(CO)CC5)n3)S(=O)CC4)CC2)nc1-c1ccccc1]

Starting from (IV-2) (see 2.4) and (V-9) (see 36.1) Example 1.37 may be prepared and purified analogously to Example 1.14 (see 14.). Analytical HPLC-MS (method B): RT=1.33 min.

### 38. Synthesis of: (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one (Example 1.38)

### 38.1 tert.butyl 4-(4,5-diphenyloxazol-2-yl)-piperidine-1-carboxylate

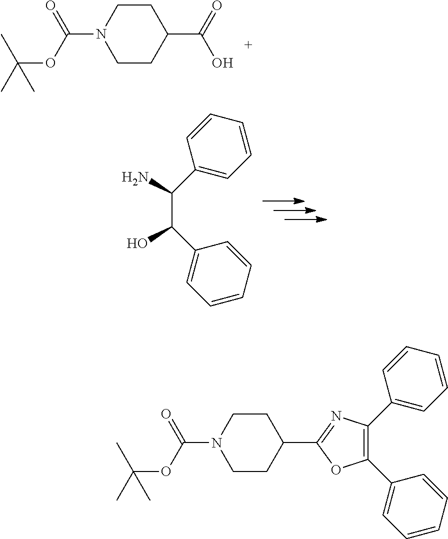 [SMILES: CC(C)(C)OC(=O)N1CCC(C(=O)O)CC1 ; N[C@@H](c1ccccc1)[C@H](O)c1ccccc1 ; CC(C)(C)OC(=O)N1CCC(c2nc(-c3ccccc3)c(-c3ccccc3)o2)CC1]

Starting from 1.08 g mono-tert-butylpiperidine-1,4-dicarboxylate and 1 g 2-amino-1,2-diphenyl-ethanol the product may be prepared as described in the literature (see Tet. 2001, 4867). The product is purified by chromatography (method B). 560 mg are obtained as an oil. Analytical HPLC-MS (method A): RT=1.72 min.

### 38.2 4-(4,5-diphenyloxazol-2-yl)-piperidine (V-10)

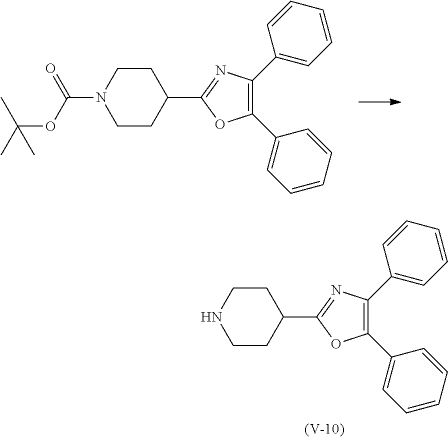 [SMILES: CC(C)(C)OC(=O)N1CCC(c2nc(-c3ccccc3)c(-c3ccccc3)o2)CC1 ; c1ccc(-c2nc(C3CCNCC3)oc2-c2ccccc2)cc1 ; "(V-10)"]

560 mg tert.butyl 4-(4,5-diphenyloxazol-2-yl)-piperidine-1-carboxylate are placed in 2 ml dichloromethane, then 1.1 ml trifluoroacetic acid are added. The reaction mixture is stirred for 15 hours at ambient temperature, then evaporated to dryness. The residue is combined with toluene and evaporated to dryness again. The residue is combined with diethyl ether and the precipitated solid is suction filtered and dried. 510 mg (V-10) are obtained. Analytical HPLC-MS (method B): RT=1.38 min.

### 38.3 (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one (Example 1.38)

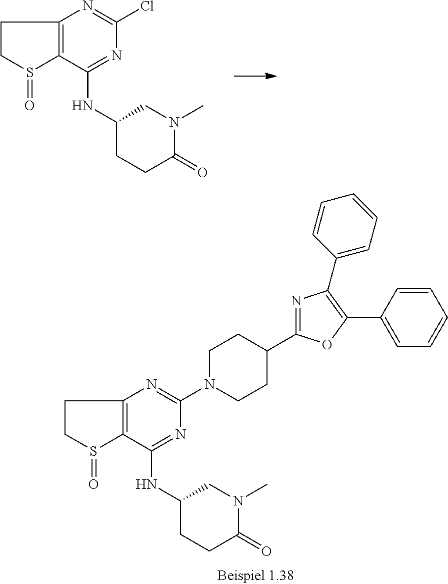 [SMILES: CN1C[C@@H](Nc2nc(Cl)nc3c2S(=O)CC3)CCC1=O ; CN1C[C@@H](Nc2nc(N3CCC(c4nc(-c5ccccc5)c(-c5ccccc5)o4)CC3)nc3c2S(=O)CC3)CCC1=O]

Starting from (IV-5) (see 5.5) and (V-10) Example 1.38 may be prepared and purified analogously to Example 1.14 (see 14.). Analytical HPLC-MS (method B): RT=1.40 min.

### 39. Synthesis of: {4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-yl}-methanol (Example 1.39)

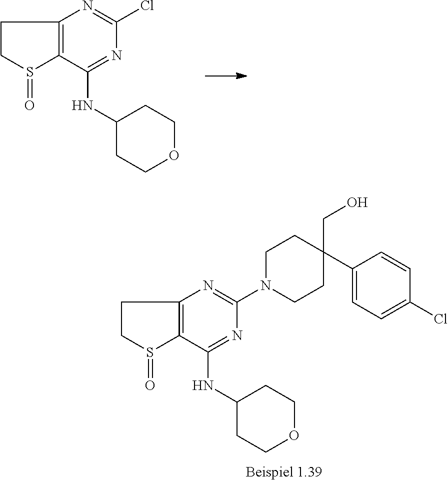 [SMILES: O=S1CCc2nc(Cl)nc(NC3CCOCC3)c21 ; O=S1CCc2nc(N3CCC(CO)(c4ccc(Cl)cc4)CC3)nc(NC3CCOCC3)c21]

Starting from (IV-6) (see 6.2) and [4-(4-chlorophenyl)-piperidin-4-yl]-methanol (J. Med. Chem. 2004, 497) Example 1.39 may be prepared and purified analogously to Example 1.15 (see 15.). Analytical HPLC-MS (method B): RT=1.24 min.

### 40. Synthesis of: [1-(2-{4[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol (Example 1.40)

### 40.1 4-[5-(4-chlorophenyl)-4-methyloxazol-2-yl]-piperidine (V-11)

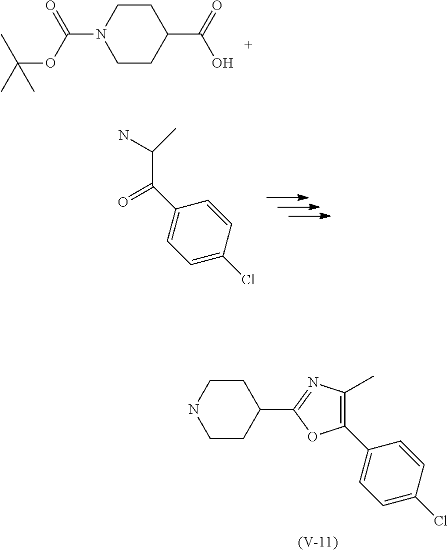 [SMILES: CC(C)(C)OC(=O)N1CCC(C(=O)O)CC1 ; CC(N)C(=O)c1ccc(Cl)cc1 ; Cc1nc(C2CCNCC2)oc1-c1ccc(Cl)cc1 ; "(V-11)"]

Starting from mono-tert-butyl piperidine-1,4-dicarboxylate and 2-amino-1-(4-chlorophenyl)-propane-1-one (see J. Med. Chem. 1974, 416) (V-11) may be prepared analogously to (V-10) (see 38.2). Analytical HPLC-MS (method B): RT=1.30 min.

### 40.2 [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol (Example 1.40)

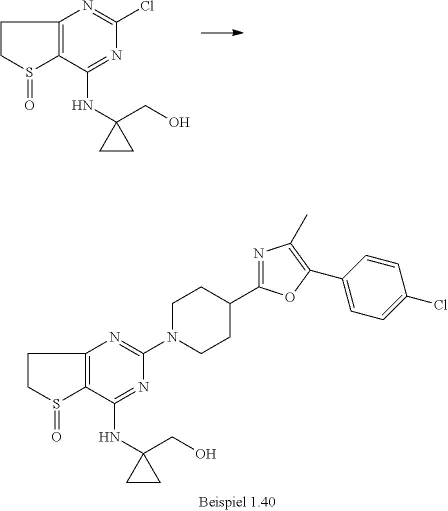 [SMILES: O=S1CCc2nc(Cl)nc(NC3(CO)CC3)c21 ; Cc1nc(C2CCN(c3nc4c(c(NC5(CO)CC5)n3)S(=O)CC4)CC2)oc1-c1ccc(Cl)cc1]

Starting from (IV-2) (see 2.4) and (V-11) Example 1.40 may be prepared and purified analogously to Example 1.14 (see 14.). Analytical HPLC-MS (method B): RT=1.37 min.

### 41. Synthesis of: 4-(4-Chlorophenyl)-1-[5-oxo-4-(tetrahydropyran-4-ylamino)-6,7-dihydro-5H-5λ⁴-thieno[3,2-d]pyrimidin-2-yl]-piperidin-4-ol (Example 1.41)

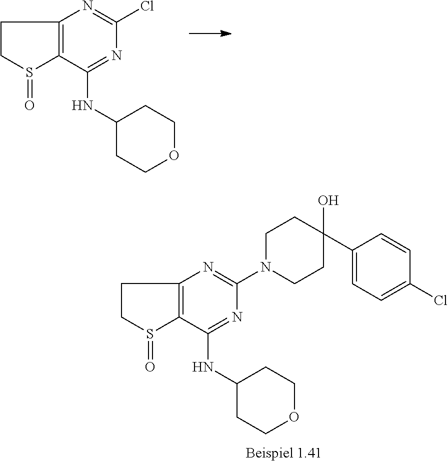 [SMILES: O=S1CCc2nc(Cl)nc(NC3CCOCC3)c21 ; O=S1CCc2nc(N3CCC(O)(c4ccc(Cl)cc4)CC3)nc(NC3CCOCC3)c21]

Starting from (IV-6) (see 6.2) and 4-(4-chlorophenyl)-piperidin-4-ol Example 1.41 may be prepared and purified analogously to Example 1.15 (see 15.). Analytical HPLC-MS (method B): RT=1.25 min.

### 42. Synthesis of: {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine (Example 1.42)

### 42.1 tert.butyl 4-(4-chlorophenyl)-4-hydroxypiperidine-1-carboxylate

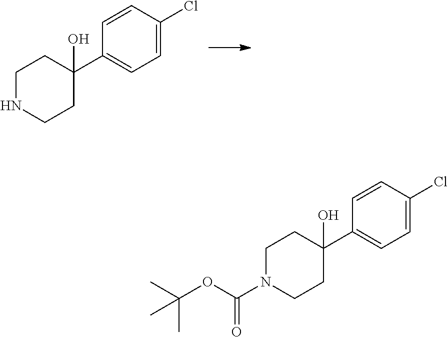 [SMILES: OC1(c2ccc(Cl)cc2)CCNCC1 ; CC(C)(C)OC(=O)N1CCC(O)(c2ccc(Cl)cc2)CC1]

500 mg 4-(4-chlorophenyl)-piperidin-4-ol are placed in 6 ml dioxane, then 0.9 ml of water and 400 mg sodium carbonate are added. After 5 min 530 mg di-tert-butyl-dicarbonate are added. The reaction mixture is stirred for 12 hours at ambient temperature, then mixed with water and the product is extracted with dichloromethane. 790 mg product are obtained as an oil. Analytical HPLC-MS (method B): RT=1.65 min.

### 42.2 tert.butyl 4-(4-chlorophenyl)-4-methoxypiperidine-1-carboxylate

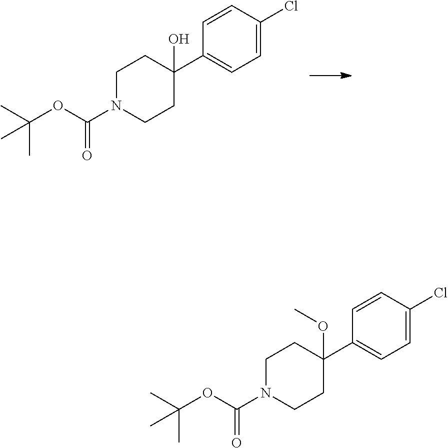 [SMILES: CC(C)(C)OC(=O)N1CCC(O)(c2ccc(Cl)cc2)CC1 ; COC1(c2ccc(Cl)cc2)CCN(C(=O)OC(C)(C)C)CC1]

790 mg tert.butyl 4-(4-chlorophenyl)-4-hydroxypiperidine-1-carboxylate are placed in 5 ml dimethylformamide and 193 mg sodium hydride (60% in mineral oil) are added. The reaction mixture is stirred for 30 min at ambient temperature, then 267 μl methyl iodide are added. After 1 h the reaction mixture is poured onto ice and the product is extracted with diethyl ether. 650 mg product are obtained as an oil. Analytical HPLC-MS (method B): RT=1.88 min.

### 42.3 4-(4-chlorophenyl)-4-methoxypiperidine (V-12)

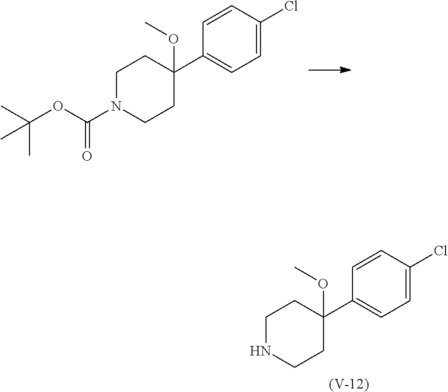

650 mg tert.butyl 4-(4-chlorophenyl)-4-methoxypiperidine-1-carboxylate are placed in 3 ml dichloromethane, then 1.46 ml trifluoroacetic acid are added. The reaction mixture is stirred overnight at ambient temperature and evaporated to dryness. The residue is combined with toluene and evaporated to dryness once more. The residue is triturated with diethyl ether and the solid is suction filtered. 450 mg (V-12) are obtained as the trifluoroacetate. Analytical HPLC-MS (method B): RT=1.22 min.

### 42.4 {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-O-amine (Example 1.42)

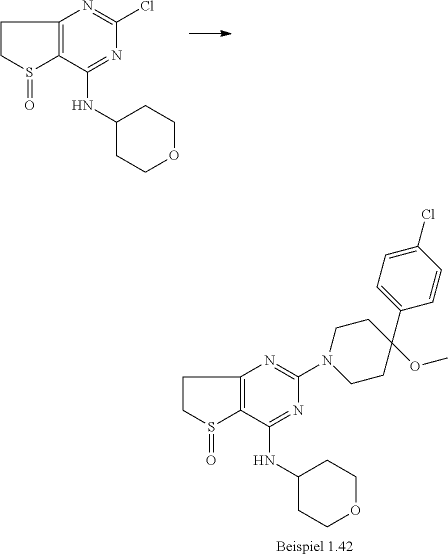 [SMILES: O=S1CCc2nc(Cl)nc(NC3CCOCC3)c21 ; COC1(c2ccc(Cl)cc2)CCN(c2nc3c(c(NC4CCOCC4)n2)S(=O)CC3)CC1]

Starting from (IV-6) (see 6.2) and (V-12) Example 1.42 may be prepared and purified analogously to Example 1.15 (see 15.). Analytical HPLC-MS (method B): RT=1.39 min.

### 43. Synthesis of: 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile (Example 1.43)

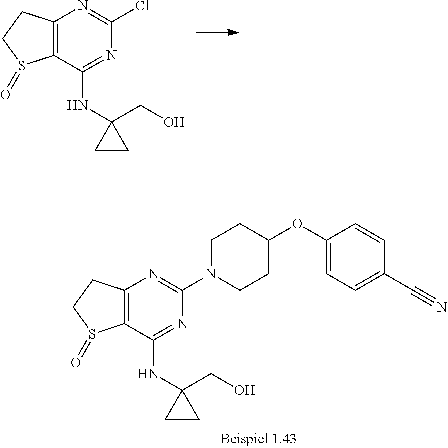 [SMILES: O=S1CCc2nc(Cl)nc(NC3(CO)CC3)c21 ; N#Cc1ccc(OC2CCN(c3nc4c(c(NC5(CO)CC5)n3)S(=O)CC4)CC2)cc1]

Starting from (IV-2) (see 2.4) and 4-(piperidin-4-yloxy)-benzonitrile (WO2007/106705) Example 1.43 may be prepared and purified analogously to Example 1.14 (see 14.). Analytical HPLC-MS (method B): RT=1.24 min.

### 44. Synthesis of: 5-oxo-2-[4-(4,5,6,7-tetrahydrobenzoxazol-2-yl)-Piperidin-1-yl]-6,7-dihydro-5H-5λ⁴-thieno[3,2-d]pyrimidin-4-yl)-(tetrahydro pyran-4-yl)-amine (Example 1.44)

### 44.1 2-(1-benzylpiperidin-4-yl)-4,5,6,7-tetrahydrobenzoxazole

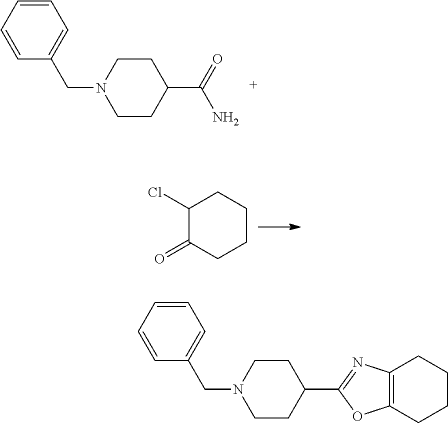

A mixture of 2.43 g 2-chlorocyclohexanone and 1 g 1-benzylpiperidine-4-carboxylic acid amide (WO2005/61483) is heated to 160° C. in the microwave until there is no further reaction. The product is purified by chromatography. 963 mg of the product are obtained. Analytical HPLC-MS (method B): RT=1.28 min.

### 44.2 2-piperidin-4-yl-4,5,6,7-tetrahydrobenzoxazole (V-13)

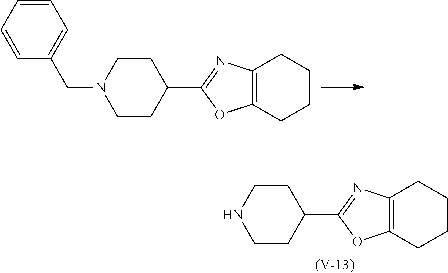

903 mg 2-(1-benzyl-piperidin-4-yl)-4,5,6,7-tetrahydrobenzoxazole are placed in 20 ml of methanol and hydrogenated with 450 mg Pd/C 10% at a pressure of 3 bar and at ambient temperature. After 12 hours the catalyst is suction filtered and the filtrate is evaporated to dryness. The product is purified by chromatography. 469 mg (V-13) are obtained as the trifluoroacetate. Analytical HPLC-MS (method B): RT=1.09 min.

### 44.3 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl)-(tetrahydropyran-4-yl)-amine (Example 1.44)

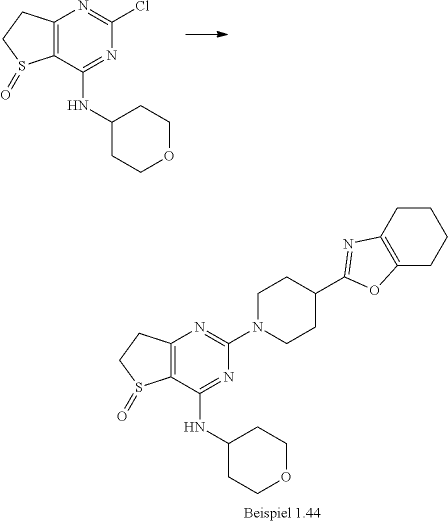 [SMILES: O=S1CCc2nc(Cl)nc(NC3CCOCC3)c21 ; O=S1CCc2nc(N3CCC(c4nc5c(o4)CCCC5)CC3)nc(NC3CCOCC3)c21]

Starting from (IV-6) (see 6.2) and (V-13) Example 1.44 may be prepared and purified analogously to Example 1.14 (see 14.). Analytical HPLC-MS (method B): RT=1.23 min.

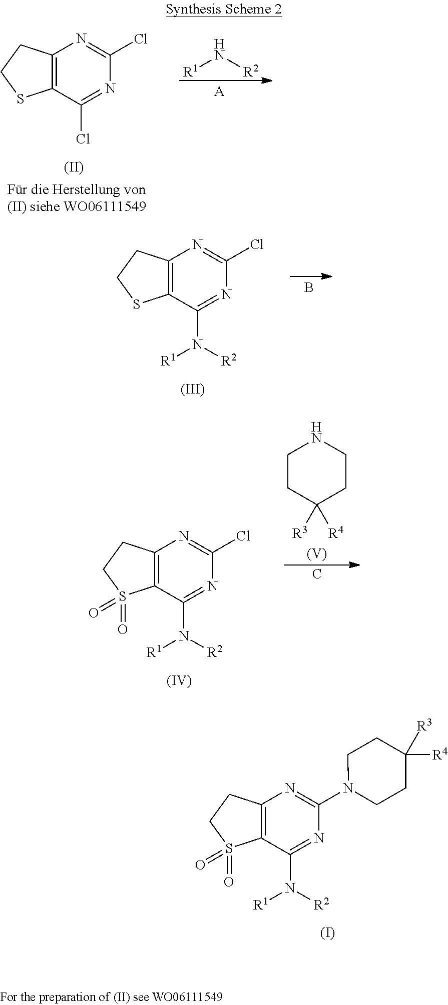

### 45. Synthesis of: (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one (Example 1.45)

### 45.1 5-(2-chloro-5,5-dioxo-6,7-dihydro-5H-5λ⁶-thieno[3,2-d]pyrimidin-4-ylamino)-1-methylpiperidin-2-one (VI-1)

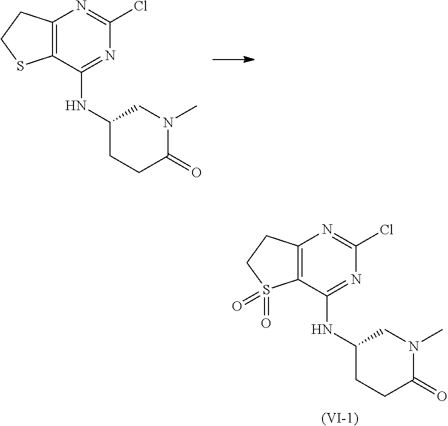 [SMILES: CN1C[C@@H](Nc2nc(Cl)nc3c2SCC3)CCC1=O ; CN1C[C@@H](Nc2nc(Cl)nc3c2S(=O)(=O)CC3)CCC1=O ; "(VI-1)"]

200 mg (III-5) (see 5.4) are placed in 3 ml trifluoroacetic acid, then 165 μl hydrogen peroxide (35%) are slowly added dropwise. An exothermic reaction takes place. The reaction mixture is stirred for 12 hours at ambient temperature, then combined with ice water and made basic with NH₄OH. The product is extracted with dichloromethane. 150 mg (VI-1) are obtained as a solid.

### 45.2 (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one (Example 1.45)

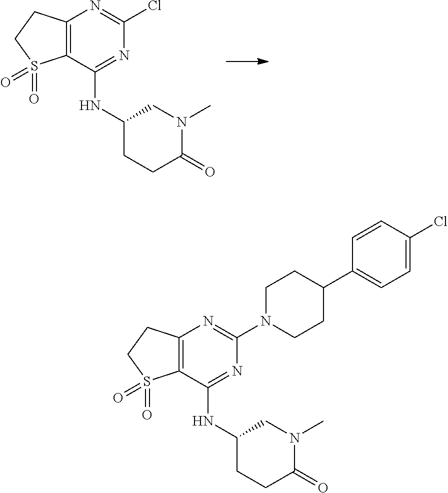 [SMILES: CN1C[C@@H](Nc2nc(Cl)nc3c2S(=O)(=O)CC3)CCC1=O ; CN1C[C@@H](Nc2nc(N3CCC(c4ccc(Cl)cc4)CC3)nc3c2S(=O)(=O)CC3)CCC1=O]

Starting from (VI-1) and 4-(4-chlorophenyl)-piperidine hydrochloride Example 1.45 may be prepared and purified analogously to Example 1.14 (see 14.). Analytical HPLC-MS (method B): RT=1.48 min.

### Methods of Chromatography

The Example compounds prepared according to the synthesis schemes shown above were characterised by the following chromatographic methods, which—if used—are individually specified in Table B, D and E.

### Analytical HPLC-MS, Method A

Waters ZMD mass spectrometer (positive ionisation (ESI+)), Alliance 2690/2695 HPLC (diode array detector, wavelength range: 210 to 500 nm), Waters 2700 Autosampler, Waters 996/2996.

A: water with 0.10% TFA
B: acetonitrile with 0.10% TFA

| time in min | % A | % B | flow rate in ml/min |
|---|---|---|---|
| 0.00 | 95 | 5 | 2.50 |
| 0.20 | 95 | 5 | 2.50 |
| 1.50 | 2 | 98 | 2.50 |
| 1.70 | 2 | 98 | 2.50 |
| 1.90 | 95 | 5 | 2.50 |
| 2.20 | 95 | 5 | 2.50 |

The stationary phase used is a Merck Chromolith™ Flash RP-18e column, 4.6 mm×25 mm (column temperature: constant at 25° C.).

### Analytical HPLC-MS, Method B

Waters ZMD mass spectrometer (positive ionisation (ESI+)), Alliance 2690/2695 HPLC (diode array detector, wavelength range: 210 to 500 nm), Waters 2700 Autosampler, Waters 996/2996.

A: water with 0.10% NH₃
B: acetonitrile with 0.10% NH₃

| time in min | % A | % B | flow rate in ml/min |
|---|---|---|---|
| 0.00 | 95 | 5 | 2.80 |
| 0.30 | 95 | 5 | 2.80 |
| 1.60 | 2 | 98 | 2.80 |
| 1.90 | 2 | 98 | 2.80 |
| 2.00 | 95 | 5 | 2.50 |

The stationary phase used is a Merck Chromolith™ Flash RP-18e column, 3 mm×100 mm (column temperature: constant at 25° C.).

### Analytical HPLC-MS, Method C

Waters ZQ2000 mass spectrometer (positive ionisation (ESI+)), HP1100 HPLC (DAD, wavelength range: 210 to 500 nm), and Gilson 215 Autosampler.

A: water with 0.10% TFA
B: acetonitrile with 0.10% TFA

| time in min | % A | % B | flow rate in ml/min |
|---|---|---|---|
| 0.00 | 95 | 5 | 1.50 |
| 2.00 | 0 | 100 | 1.50 |
| 2.50 | 0 | 100 | 1.50 |
| 2.60 | 95 | 5 | 1.50 |

The stationary phase used is a Sunfire C18 column, 4.6×50 mm, 3.5 μm, column temperature 40° C.

### Analytical HPLC-MS, Method D

Waters ZMD mass spectrometer (positive ionisation (ESI+)), Alliance 2690/2695 HPLC (diode array detector, wavelength range: 210 to 500 nm), Waters 2700 Autosampler, Waters 996/2996.

A: water with 0.10% NH₃
B: acetonitrile with 0.10% NH₃

| time in min | % A | % B | flow rate in ml/min |
|---|---|---|---|
| 0.00 | 95 | 5 | 3.00 |
| 0.20 | 95 | 5 | 3.00 |
| 1.50 | 2 | 98 | 3.00 |
| 1.90 | 2 | 98 | 3.00 |
| 2.00 | 2 | 98 | 3.00 |

The stationary phase used is Waters, X-Bridge, C18, 3.5 nm, 4.6×20 mm. ambient temperature.

### Analytical HPLC-MS, Method E

Waters ZMD mass spectrometer (positive ionisation (ESI+)), Alliance 2690/2695 HPLC (diode array detector, wavelength range: 210 to 500 nm), Waters 2700 Autosampler, Waters 996/2996.

A: water with 0.10% TFA
B: acetonitrile with 0.10% TFA

| time in min | % A | % B | flow rate in ml/min |
|---|---|---|---|
| 0.00 | 95 | 5 | 1.20 |
| 0.30 | 95 | 5 | 1.20 |
| 9.00 | 2 | 98 | 1.20 |
| 9.40 | 2 | 98 | 1.20 |
| 9.50 | 95 | 5 | 2.80 |
| 9.90 | 95 | 5 | 2.80 |
| 10.00 | 95 | 5 | 0.20 |

The stationary phase used is a Merck Chromolith™ Flash RP-18e column, 4.6 mm×25 mm (column temperature: constant at 25° C.).

### Analytical HPLC, Method A

Agilent 1100 (diode array detection, wavelength range: 210-380 nm).

A: water with 0.10% TFA
B: acetonitrile with 0.13% TFA

| time in min | % A | % B | flow rate in ml/min |
|---|---|---|---|
| 0.00 | 95 | 5 | 1.50 |
| 0.60 | 95 | 5 | 1.50 |
| 3.40 | 2 | 98 | 1.50 |
| 3.90 | 2 | 98 | 1.50 |
| 4.20 | 95 | 5 | 1.50 |
| 4.90 | 95 | 5 | 1.50 |

The stationary phase used is a Varian Microsorb column, RP C18, 3 μm, 100 A, ambient temperature.

### Preparative HPLC-MS, Method A

Waters ZQ2000 mass spectrometer (positive ionisation (ESI+)), HP1100 HPLC (DAD, wavelength range: 210-500 nm), and Gilson 215 Autosampler.

A: water with 0.10% TFA
B: acetonitrile

| time in min | % A | % B | flow rate in ml/min |
|---|---|---|---|
| 0.00 | 90 | 10 | 50 |
| 1.50 | 90 | 10 | 50 |
| 8.00 | 40 | 60 | 50 |
| 10.00 | 40 | 60 | 50 |
| 11.00 | 90 | 10 | 50 |

The stationary phase used is a Sunfire C18 column, 30×100 mm, 5 μm, ambient temperature.

### Preparative HPLC, Method A

Gilson HPLC with Gilson UV-VIS-155 Detector, Sampling Injector 231 XL.

The wavelength stated is the substance-specific UV maximum.

A: water with 0.13% TFA
B: acetonitrile with 0.1% TFA

| time in min | % A | % B | flow rate in ml/min |
|---|---|---|---|
| 0.00 | 95 | 5 | 165 |
| 1.30 | 95 | 5 | 165 |
| 8.90 | 2 | 98 | 165 |
| 10.00 | 2 | 98 | 165 |
| 10.50 | 95 | 5 | 165 |
| 11.60 | 95 | 5 | 165 |

The stationary phase used is a Microsorb RP 18 column, 8 μm, 50×65 mm, ambient temperature.

### Preparative HPLC, Method B

Gilson HPLC with Gilson UV-VIS-155 Detector, Sampling Injector 231 XL.

The wavelength stated is the substance-specific UV maximum.

A: water with 0.1% ammonia 35%
B: acetonitrile

| time in min | % A | % B | flow rate in ml/m |
|---|---|---|---|
| 0.00 | 95 | 5 | 180 |
| 1.40 | 95 | 5 | 180 |
| 17.00 | 2 | 98 | 180 |
| 18.50 | 2 | 98 | 180 |
| 18.70 | 95 | 5 | 180 |
| 20.-50 | 95 | 5 | 180 |

The stationary phase used is a Pursuit XRS RP 18 column, 10 μm, 50×150 mm, ambient temperature.

### Preparative HPLC, Method C

Gilson HPLC with Gilson UV-VIS-155 Detector, Sampling Injector 231 XL.

The wavelength stated is the substance-specific UV maximum.

A: water with 0.13% TFA
B: acetonitrile with 0.1% TFA

| time in min | % A | % B | flow rate in ml/min |
|---|---|---|---|
| 0.00 | 95 | 5 | 180 |
| 1.40 | 95 | 5 | 180 |
| 17.00 | 2 | 98 | 180 |
| 18.50 | 2 | 98 | 180 |
| 18.70 | 95 | 5 | 180 |
| 20.50 | 95 | 5 | 180 |

The stationary phase used is a Microsorb RP 18 column, 8 μm, 50×150 mm, ambient temperature.

### Preparative HPLC, Method D

Gilson HPLC with Gilson UV-VIS-155 Detector, Sampling Injector 231 XL.

The wavelength stated is the substance-specific UV maximum.

A: water with 0.1% ammonia 35%
B: acetonitrile

| time in min | % A | % B | flow rate in ml/min |
|---|---|---|---|
| 0.00 | 95 | 5 | 180 |
| 1.10 | 95 | 5 | 180 |
| 9.00 | 2 | 98 | 180 |
| 10.00 | 2 | 98 | 180 |
| 10.50 | 95 | 5 | 180 |
| 12.00 | 95 | 5 | 180 |

The stationary phase used is a X-Bridge C18 column, 5 μm, 50×65 mm, ambient temperature.

### Indications

As has been found, the combinations according to the invention containing a PDE4 inhibitor, preferably a PDE4 inhibitor of formula 1 and at least one EP4 receptor antagonist are characterised by their wide range of applications in the therapeutic field. Particular mention should be made of those applications for which the combinations according to the invention are preferably suited on account of their pharmaceutical efficacy as PDE4 inhibitors. Examples include respiratory or gastrointestinal diseases or complaints, inflammatory diseases of the joints, skin or eyes, cancers, and also diseases of the peripheral or central nervous system.

Particular mention should be made of the prevention and treatment of diseases of the airways and of the lung which are accompanied by increased mucus production, inflammations and/or obstructive diseases of the airways. Examples include acute, allergic or chronic bronchitis, chronic obstructive bronchitis (COPD), coughing, pulmonary emphysema, allergic or non-allergic rhinitis or sinusitis, chronic rhinitis or sinusitis, asthma, alveolitis, Farmer's disease, hyperreactive airways, infectious bronchitis or pneumonitis, paediatric asthma, bronchiectases, pulmonary fibrosis, ARDS (acute adult respiratory distress syndrome), bronchial oedema, pulmonary oedema, bronchitis, pneumonia or interstitial pneumonia triggered by various causes, such as aspiration, inhalation of toxic gases, or bronchitis, pneumonia or interstitial pneumonia as a result of heart failure, irradiation, chemotherapy, cystic fibrosis or mucoviscidosis, or alpha1-antitrypsin deficiency.

Also deserving special mention is the treatment of inflammatory diseases of the gastrointestinal tract. Examples include acute or chronic inflammatory changes in gall bladder inflammation, Crohn's disease, ulcerative colitis, inflammatory pseudopolyps, juvenile polyps, colitis cystica profunda, pneumatosis cystoides intestinales, diseases of the bile duct and gall bladder, e.g. gallstones and conglomerates, for the treatment of inflammatory diseases of the joints such as rheumatoid arthritis or inflammatory diseases of the skin and eyes.

Preferential mention should also be made of the treatment of cancers. Examples include all forms of acute and chronic leukaemias such as acute lymphatic and acute myeloid leukaemia, chronic lymphatic and chronic myeloid leukaemia, and bone tumours such as osteosarcoma and all types of glioma such as oligodendroglioma and glioblastoma.

Preferential mention should also be made of the prevention and treatment of diseases of the peripheral or central nervous system. Examples of these include depression, bipolar or manic depression, acute and chronic anxiety states, schizophrenia, Alzheimer's disease, Parkinson's disease, acute and chronic multiple sclerosis or acute and chronic pain as well as injuries to the brain caused by stroke, hypoxia or craniocerebral trauma.

Particularly preferably the present invention relates to the use of the combinations according to the invention for preparing a medicament for the treatment of inflammatory or obstructive diseases of the upper and lower respiratory tract including the lungs, such as for example allergic rhinitis, chronic rhinitis, bronchiectasis, cystic fibrosis, idiopathic pulmonary fibrosis, fibrosing alveolitis, COPD, chronic bronchitis, chronic sinusitis, asthma, Crohn's disease, ulcerative colitis, particularly COPD, chronic bronchitis and asthma.

Also preferred is the use of the combinations according to the invention for the treatment of diseases of the peripheral or central nervous system such as depression, bipolar or manic depression, acute and chronic anxiety states, schizophrenia, Alzheimer's disease, Parkinson's disease, acute and chronic multiple sclerosis, amyotropic lateral sclerosis (ALS) or acute and chronic pain conditions and brain damage caused by stroke, hypoxia or cerebro-cranial trauma.

It is most preferable to use the combinations according to the invention for the treatment of inflammatory and obstructive diseases such as COPD, chronic bronchitis, chronic sinusitis, asthma, Crohn's disease, ulcerative colitis, particularly COPD, chronic bronchitis and asthma.

An outstanding aspect of the formulations according to the invention containing a combination of one (or more) PDE4 inhibitors, preferably the PDE4 inhibitors of formula 1, and at least one EP4 receptor antagonist is their reduced profile of side effects compared with formulations that contain the same PDE4 inhibitors in the same amount in the absence of an EP4 receptor antagonist. Side effects that frequently occur when taking a PDE4 inhibitor preferentially include, inter alia, diarrhoea, nausea and vomiting. In the rat model further side effects were observed after the administration of PDE4 inhibitor, such as for example weight loss, loss of spleen weight, leukocytosis and neutrophilia, diarrhoea and the occurrence of mesenteric vasculitis.

By a reduced profile of side effects is meant, within the scope of the invention, in particular being able to administer a therapeutically effective dose of a PDE4 inhibitor in a pharmaceutical composition according to the invention without inducing to any appreciable extent in the patient the or at least one of the side effects commonly observed when PDE4 inhibitors are administered (diarrhoea, nausea, vomiting, weight loss, loss of spleen weight, leukocytosis and neutrophilia and the occurrence of mesenteric vasculitis). It is particularly preferable to administer a therapeutically effective amount of a PDE4 inhibitor in the composition according to the invention at every stage of the course of the disease without triggering the typical PDE4 inhibitor-mediated side effects of diarrhoea, weight loss, leukocytosis, neutrophilia or mesenteric vasculitis. In a particular aspect the present invention relates to the administration of a therapeutically effective amount of the pharmaceutical composition according to the invention at every stage of the course of the disease without triggering the typical PDE4 inhibitor-mediated side effect of mesenteric vasculitis to any appreciable degree.

Experiments on the rat model described hereinafter show that the pharmaceutical compositions according to the invention containing a PDE4 inhibitor and at least one EP4 receptor antagonist substantially reduce or even totally prevent many of the side effects which occur when the corresponding PDE4 inhibitor is administered on its own.

### EXPERIMENTAL METHOD

### Experiment 1

### Diclofenac Provides Protection Against Roflumilast-Mediated Effects Such as Weight Loss, Loss of Spleen Weight, Neutrophilia and Mesenteric Vasculitis

Six male Wistar rats in each group were treated for four days with the following substances (all substances are given p.o.=orally):

Group 1 (“Control Group”):

Six male Wistar rats were given a daily dose of 0.5% Natrosol (placebo) at the times 0800, 1300 and 1700 hours.

Group 2 (“Diclofenac Group”):

Six male Wistar rats were given a daily dose of 1 mg/kg of diclofenac (NSAID) at the times 0800 and 1700 hours and 0.5% Natrosol (placebo) at 1300 hours.

Group 3 (“Roflumilast Group”):

Six male Wistar rats were given a daily dose of 0.5% Natrosol (placebo) at 0800 and 1700 hours and 10 mg/kg roflumilast (PDE4 inhibitor) at 1300 hours.

Group 4 (“Roflumilast+Diclofenac Group”):

Six male Wistar rats were given a daily dose of 1 mg/kg diclofenac (NSAID) at the times 0800 and 1700 hours and 10 mg/kg roflumilast (PDE4 inhibitor) at 1300 hours.

For pharmacokinetic analysis (determining the plasma levels of the substances) on day 4 one rat from each group was used; these rats were no longer available for other parameters under investigation. The same applied to one rat from the roflumilast group which died between day 4 and day 5 of the experiment.

During the experiment the body weights of the animals were determined and the differences in the body weights of the rats from the different groups towards the end of the experiment were shown as a percentage change from the time of the first administration (=day 1, 0800 hours (=time t₀)). The average±standard deviation of the body weights at time t₀ was 355±17 g.

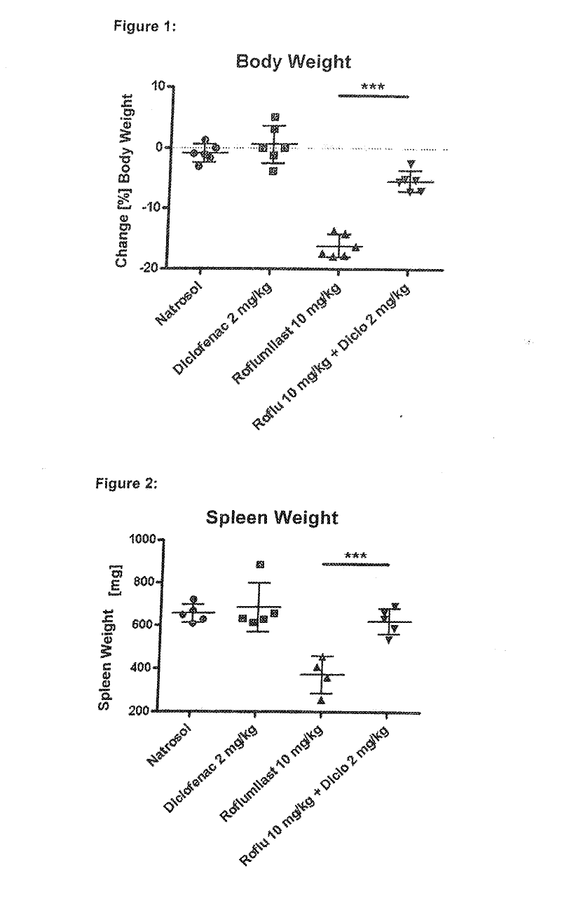

At the end of the experiment (day 5, 95 hours after t₀ (=the time of the first administration on day 1, 0800)) the proportion of neutrophils (in % of white blood cells, FIG. 2) were determined from the blood of 4 or 5 of the rats from the individual groups. In addition, the weights of the animals' spleens were measured and the mesenteries were dissected for histopathological investigation for vasculitis (multifocal perivascular mononuclear/polymorphonuclear infiltration).

FIG. 1 shows the changes in the body weight measured after the respective administrations to the control (Natrosol) group, the diclofenac group, the roflumilast group and the roflumilast+diclofenac group (statistics: Two-way analysis of variance; ***=p<0.001).

FIG. 2 shows the changes in the spleen weight measured after the respective administrations to the control (Natrosol) group, the diclofenac group, the roflumilast group and the roflumilast+diclofenac group (statistics: Two-way analysis of variance; ***=p<0.001).

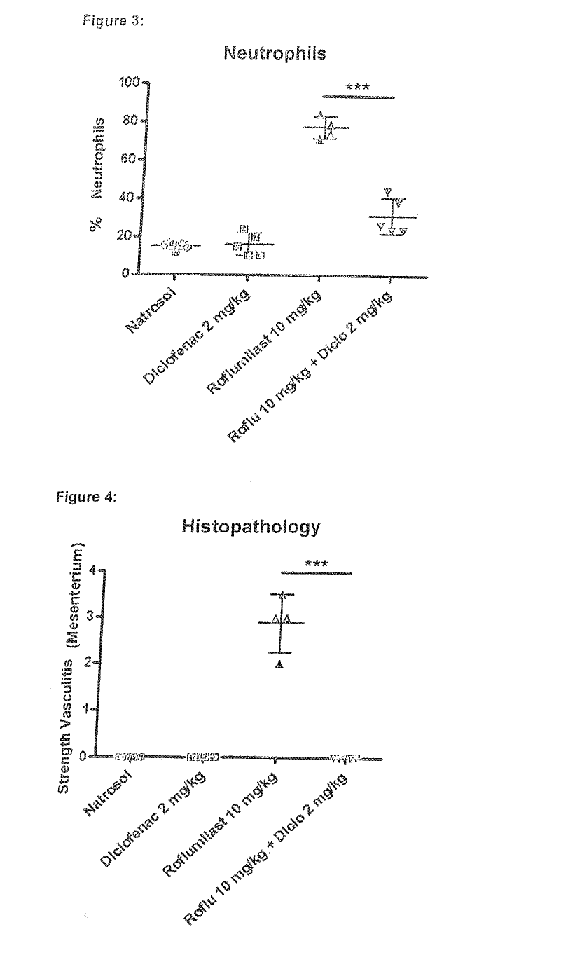

FIG. 3 shows the percentage of neutrophils in the blood after the respective administrations to the control (Natrosol) group, the diclofenac group, the roflumilast group and the roflumilast+diclofenac group (statistics: Two-way analysis of variance; ***=p<0.001).

FIG. 4 shows the increase in the vasculitis observed in the mesentery after the respective administrations to the control (Natrosol) group, the diclofenac group, the roflumilast group and the roflumilast+diclofenac group (statistics: Two-way analysis of variance; ***=p<0.001).

To summarise, it can be stated that the PDE4 inhibitor-mediated side effects observed in the Roflumilast group such as loss of body weight (FIG. 1), loss of spleen weight (FIG. 2), neutrophilia (FIG. 3) and mesenteric vasculitis (monocyte/polymorphonuclear infiltration into the mesentery, FIG. 4) can be substantially reduced or prevented (may even be reduced almost to the levels for the control group), if an NSAID such as for example diclofenac is co-administered simultaneously or only offset by a few hours (see Roflumilast+Diclofenac group). The parameters measured after the administration of diclofenac on its own appeared to be very similar to the control groups.

On the one hand, during the treatment of COPD and asthma patients with a PDE4 inhibitor the simultaneous administration of an NSAID such as for example diclofenac therefore has major advantages, as the NSAID can substantially reduce or even prevent the PDE4-receptor-mediated side effects. On the other hand, it has long been known that NSAIDs such as diclofenac when taken regularly have their own side effects, such as for example potent gastrointestinal side effects, particularly the formation of gastric ulcers. However, it would be necessary to take these NSAIDs regularly in order to reduce the PDE4-mediated side effects, as the treatment of COPD and asthma patients with PDE4-inhibitors generally indicates long-term therapy. Consequently, the question arises regarding alternatives to the PDE4 inhibitor/NSAID combined therapy which have a lower side effect profile.

### Experiment 2

### The COX-2 Selective Inhibitor Lumiracoxib, but not the COX-1 Selective Inhibitor SC-560, Protects Against Roflumilast-Mediated Effects Such as Loss of Body Weight, Loss of Spleen Weight, Neutrophilia and Mesenteric Vasculitis

Six male Wistar rats in each group were treated for four days with the following substances (all drugs were given p.o.=orally):

### Group 1 (“Control Group”):

Six male Wistar rats were given a daily dosage of 0.5% Natrosol (Placebo) at the times 0800, 1300 and 1700 hours.

### Group 2 (“SC-560 Group”):

Six male Wistar rats were given a daily dosage of in each case 2 mg/kg SC-560 (NSAID, selective for Cox-1) at the times 0800 and 1700 hours and 0.5% Natrosol at 1300 hours.

### Group 3 (“Lumiracoxib Group”):

Six male Wistar rats were given a daily dosage of in each case 2 mg/kg Lumiracoxib (NSAID, selective for Cox-2) at the times 0800 and 1700 hours and 0.5% Natrosol at 1300 hours.

### Group 4 (“Roflumilast Group”):

Six male Wistar rats were given a daily dosage of 0.5%

Natrosol (Placebo) in each case at the times 0800 and 1700 hours and 10 mg/kg Roflumilast (PDE4 inhibitor) at 1300 hours.

### Group 5 (“Roflumilast+SC-560 Group”):

Six male Wistar rats were given a daily dosage of in each case 2 mg/kg SC-560 (NSAID, selective for COX-1) at the times 0800 and 1700 hours and 10 mg/kg Roflumilast (PDE4 inhibitor) at 1300 hours.

### Group 6 (“Roflumilast+Lumiracoxib Group”):

Six male Wistar rats were given a daily dosage of in each case 2 mg/kg Lumiracoxib (NSAID, selective for COX-2) at the times 0800 and 1700 hours and 10 mg/kg Roflumilast (PDE4 inhibitor) at 1300 hours. For pharmacokinetic analysis (determining plasma levels of the substances) one rat from each group was used on day 4; these rats were no longer available for the investigation of other parameters.

During the experiment the body weights of the animals were determined and the differences in the body weights of the rats from the different groups were given at the end of the experiment as a change in % based on the 1st administration time (=day 1, 0800 (=time t₀)). The average value±standard deviation in the body weights at time t₀ was 306±11 g. At the end of the experiment (day 5, 95 hours after t₀ (t₀=time of the first administration on day 1, 0800)) the proportion of neutrophils (in % of white blood cells, FIG. 2) were determined from the blood of 4 or 5 of the rats from the individual groups. In addition, the weights of the animals' spleens were measured and the mesenteries were dissected for histopathological investigation for vasculitis (multifocal perivascular mononuclear/polymorphonuclear infiltration).

FIG. 5 shows the change in the body weight measured after the respective administrations to the control (Natrosol) group, the SC 560 group, the Lumiracoxib group, the Roflumilast group, the Roflumilast+SC 560 group and the Roflumilast+Lumiracoxib group (statistics: One-way analysis of variance; ns=not significant; *=p<0.05; ***=p<0.001).

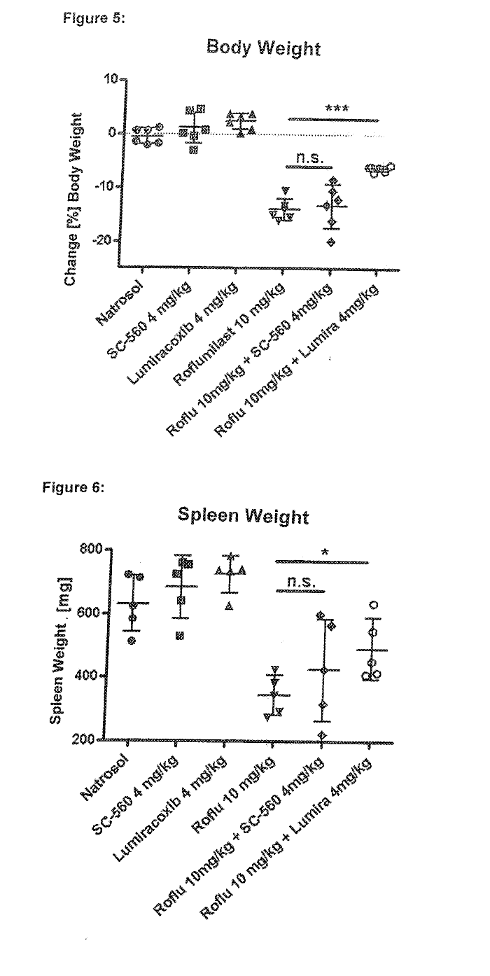

FIG. 6 shows the change in the spleen weight measured after the respective administrations to the control (Natrosol) group, the SC 560 group, the Lumiracoxib group, the Roflumilast group, the Roflumilast+SC 560 group and the Roflumilast+Lumiracoxib group (statistics: One-way analysis of variance; ns=not significant; *=p<0.05; ***=p<0.001).

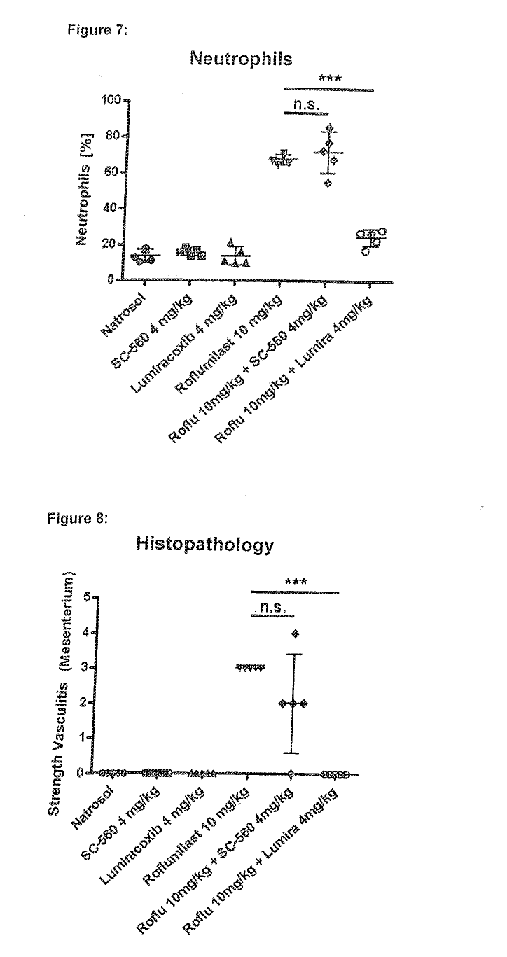

FIG. 7 shows the percentage of neutrophils measured in the blood after the respective administrations to the control (Natrosol) group, the SC 560 group, the Lumiracoxib group, the Roflumilast group, the Roflumilast+SC 560 group and the Roflumilast+Lumiracoxib group (statistics: One-way analysis of variance; ns=not significant; *=p<0.05; ***=p<0.001).

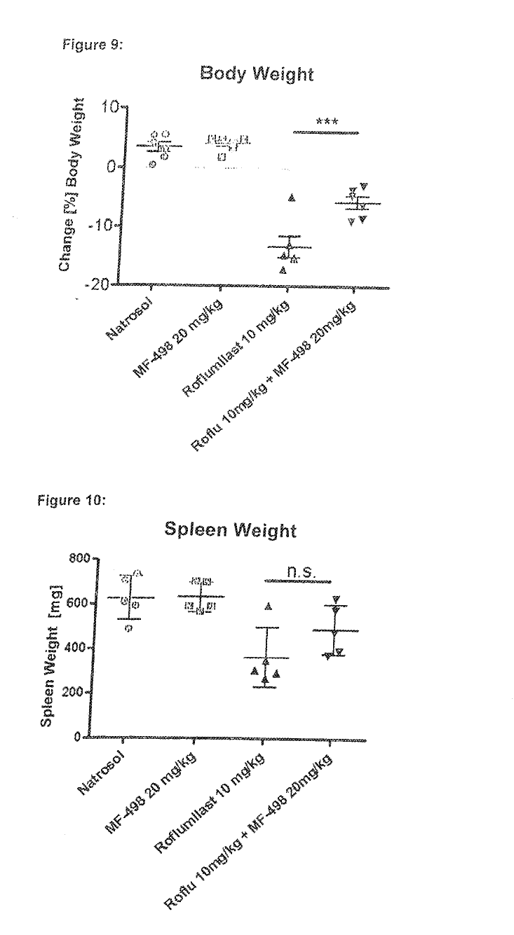

FIG. 8 shows the increase in the vasculitis observed in the mesentery after the respective administrations to the control (Natrosol) group, the SC 560 group, the Lumiracoxib group, the Roflumilast group, the Roflumilast+SC 560 group and the Roflumilast+Lumiracoxib-group (statistics: One-way analysis of variance; ns=not significant; *=p<0.05; ***=p<0.001).

To summarise, it can be stated that the PDE4 inhibitor-mediated side effects observed in the Roflumilast group such as loss of body weight (FIG. 5), loss of spleen weight (FIG. 6), neutrophilia (FIG. 7) and mesenteric vasculitis (monocyte/polymorphonuclear perivascular infiltration into the mesentery as a measurement of vasculitis, FIG. 8) can be substantially reduced or prevented (may even be reduced almost to the levels for the control group), if a COX-2-selective NSAID such as Lumiracoxib is co-administered simultaneously or only offset by a few hours (see Roflumilast+Lumiracoxib). The COX-1 selective NSAID SC-560 has no protective effect on loss of body weight, loss of spleen weight, neutrophilia and the monocyte/polymorphonuclear perivascular infiltration as a measurement of vasculitis. The parameters measured after the administration of SC-560 or Lumiracoxib on its own appeared to be very similar to the control groups.

All in all it can be concluded that the protective effect of an NSAID on the PDE4-inhibitor-mediated side effects is based on inhibiting COX-2.

On the one hand, during the treatment of COPD and asthma patients with a PDE4 inhibitor the simultaneous administration of a COX-2 inhibitor such as Lumiracoxib for example has major advantages, as the NSAID can substantially reduce or even prevent the PDE4-receptor-mediated side effects, and on the other hand, no potent gastrointestinal side effects need to be feared from the COX-2 inhibitor (as is the case with NSAIDs). On the other hand, however, it has long been known that COX-2 inhibitors such as Lumiracoxib when taken regularly have cardiovascular side effects (myocardial infarction, thromboses, stroke) (cf. Clark et al; The Journal of Pharmacology and Experimental Therapeutics; 325; p. 425-434). However, it would be necessary to take these NSAIDs regularly in order to reduce the PDE4-mediated side effects, as the treatment of COPD and asthma patients with PDE4-inhibitors generally constitutes long-term therapy. Consequently, the question arises regarding alternatives to the PDE4 inhibitor/COX-2 inhibitor combined therapy which have a lower side effect profile.

### Experiment 3

### The EP4-Receptor-Selective Inhibitor MF498 (N-{[4-(5,9-diethoxy-6-oxo-6,8-dihydro-7H-pyrrolo[3,4-g]quinolin-7-yl)-3-methylbenzyl)sulphonyl}-2-(2-methoxyphenyl)acetamide, Also Referred to as Compound 2.1) Protects Against Roflumilast-Mediated Effects Such as Loss of Body Weight, Loss of Spleen Weight, Neutrophilia and Mesenteric Vasculitis

Six male Wistar rats per group were treated for four days with the following substances (all substances are given p.o.=orally):

### Group 1 (“Control Group”):

Six male Wistar rats were given a daily dosage of 0.5% Natrosol (Placebo) at 1300 hours.

### Group 2 (“MF-498 Group”):

Six male Wistar rats were given a daily dosage of 20 mg/kg MF-498 (EP4 antagonist) in each case at 1300 hours.

### Group 3 (“Roflumilast Group”):

Six male Wistar rats were given a daily dosage of 10 mg/kg Roflumilast (PDE4 inhibitor) at 1300 hours.

### Group 4 (“Roflumilast+MF-498 Group”):

Six male Wistar rats were given a daily dosage of 20 mg/kg MF-498 (EP4 antagonist) and 10 mg/kg Roflumilast (PDE4 inhibitor) at 1300 hours. For pharmacokinetic analyses (determining the plasma levels of the substances) one rat per group was used on day 4; these rats were no longer available for other parameters under investigation.

During the experiment the body weights of the animals were determined and the differences in the body weights of the rats from the different groups at the end of the experiment were shown as a percentage change from the time of the first administration (=day 1, 0800 hours (=time t₀)). The average±standard deviation of the body weights at time t₀ was 283±6 g. At the end of the experiment (day 5, 95 hours after t₀ (=the time of the first administration on day 1, 0800)) the proportion of neutrophils (in % of white blood cells, FIG. 2) were determined from the blood of 4 or 5 of the rats from the individual groups. In addition, the weights of the animals' spleens were measured and the mesenteries were dissected for histopathological investigation for vasculitis (multifocal perivascular mononuclear/polymorphonuclear infiltration).

FIG. 9 shows the change in the body weight measured after the respective administrations to the control (Natrosol) group, the MF-498 group, the Roflumilast group and the Roflumilast+MF-498 group (statistics: Two-way analysis of variance; ns=not significant; **=p<0.01, ***=p<0.001).

FIG. 10 shows the change in the spleen weight measured after the respective administrations in the control (Natrosol) group, the MF-498 group, the Roflumilast group and the Roflumilast+MF-498 group (statistics: Two-way analysis of variance; ns=not significant; **=p<0.01, ***=p<0.001).

FIG. 11 shows the percentage of neutrophils in the blood after the respective administrations to the control (Natrosol) group, the MF-498 group, the Roflumilast group and the Roflumilast+MF-498 group (statistics: Two-way analysis of variance; ns=not significant; **=p<0.01, =p<0.001).

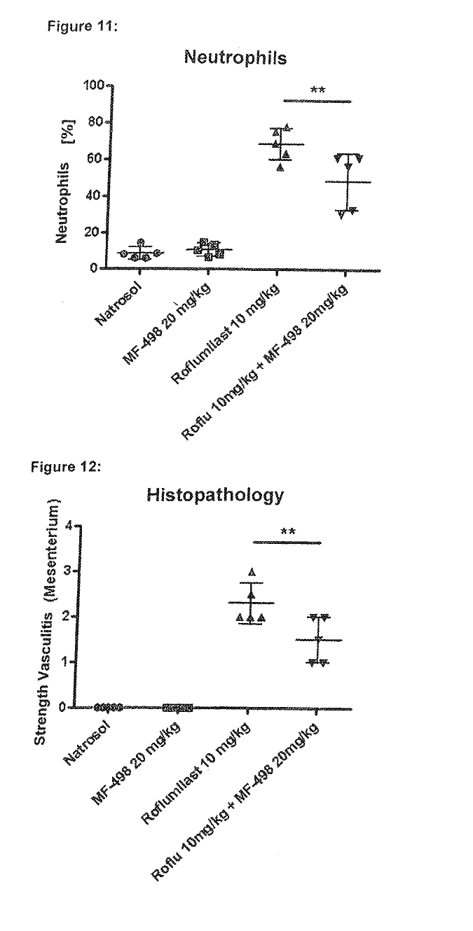

FIG. 12 shows the increase in the vasculitis observed in the mesentery after the respective administrations to the control (Natrosol) group, the MF-498 group, the Roflumilast group and the Roflumilast+MF-498 group (statistics: Two-way analysis of variance; ns=not significant; **=p<0.01, ***=p<0.001).

To summarise, it can be stated that the PDE4 inhibitor-mediated side effects observed in the Roflumilast group such as loss of body weight (FIG. 9), loss of spleen weight (FIG. 10), neutrophilia (FIG. 11) and mesenteric vasculitis (monocyte/polymorphonuclear perivascular infiltration into the mesentery as a measurement of vasculitis, FIG. 12) can be substantially reduced if a selective EP4 antagonist is co-administered simultaneously (see Roflumilast+MF-498). The differences in the weight of the spleen between the Roflumilast group and the Roflumilast+MF-498 group do not achieve statistical significance, as in this experiment an animal displayed an unusually small loss of spleen weight in the Roflumilast group. The parameters measured after the administration of MF-498 on its own appeared to be very similar to the control group.

All in all it can be concluded that the protective effect of an NSAID or a COX-2 inhibitor on the PDE4-inhibitor-mediated side effects is based at least partly on reducing the prostaglandin E2 synthesis of the COX-2 enzyme, which mediates the side effects through the EP4 receptor further on down the signal chain. Therefore the side effects can also be reduced by blocking the EP4 receptor. What is important in this context is that the EP4 receptor itself signals into the cell through an increase in the messenger molecule cAMP and cAMP is the substrate for the PDE4 enzymes that are inhibited by roflumilast. Therefore the influence of the PDE4 inhibitor is doubled, firstly resulting from the increase in the expression of cyclooxygenase-2, and concomitantly with that an increased production of prostaglandin E2 (PGE2) and at the same time an intensification of the PGE2/EP4-mediated signal (cAMP) in the affected cells as a result of the prevention of cAMP degradation.

### Experiment 4

### The EP2-Receptor-Preferential Inhibitor AH6809 (6-isopropoxy-9-oxoxanthene-2-carboxylic acid), does not Protect Against Roflumilast-Mediated Effects Such as Loss of Body Weight, Loss of Spleen Weight, Neutrophilia and Mesenteric Vasculitis

Six male Wistar rats per group were treated for four days with the following substances (all substances are given p.o.=orally):

### Group 1 (“Control Group”):

Six male Wistar rats were given a daily dosage of 0.5% Natrosol (Placebo) at 1300 hours.

### Group 2 (“AH-6809 Group”):

Six male Wistar rats were given a daily dosage of 10 mg/kg AH-6809 (preferential EP2 antagonist) at 1300 hours.

### Group 3 (“Roflumilast Group”):

Six male Wistar rats were given a daily dosage of 10 mg/kg Roflumilast (PDE4 inhibitor) at 1300 hours.

### Group 4 (“Roflumilast+AH-6809 Group”):

Six male Wistar rats were given a daily dosage of 10 mg/kg AH-6809 (preferential EP2 antagonist) and 10 mg/kg Roflumilast (PDE4 inhibitor) at 1300 hours.

For pharmacokinetic analyses (determining the plasma levels of the substances) one rat per group was used on day 4; these rats were no longer available for other parameters under investigation.

During the experiment the body weights of the animals were determined and the differences in the body weights of the rats from the different groups at the end of the experiment were shown as a percentage change from the time of the first administration (=day 1, 0800 hours (=time t₀)). The average±standard deviation of the body weights at time t₀ was 284±9 g. At the end of the experiment (day 5, 95 hours after t₀ (=the time of the first administration on day 1, 0800)) the proportion of neutrophils (in % of white blood cells, FIG. 2) were determined from the blood of 4 or 5 of the rats from the individual groups. In addition, the weights of the animals' spleens were measured and the mesenteries were dissected for histopathological investigation for vasculitis (multifocal perivascular mononuclear/polymorphonuclear infiltration).

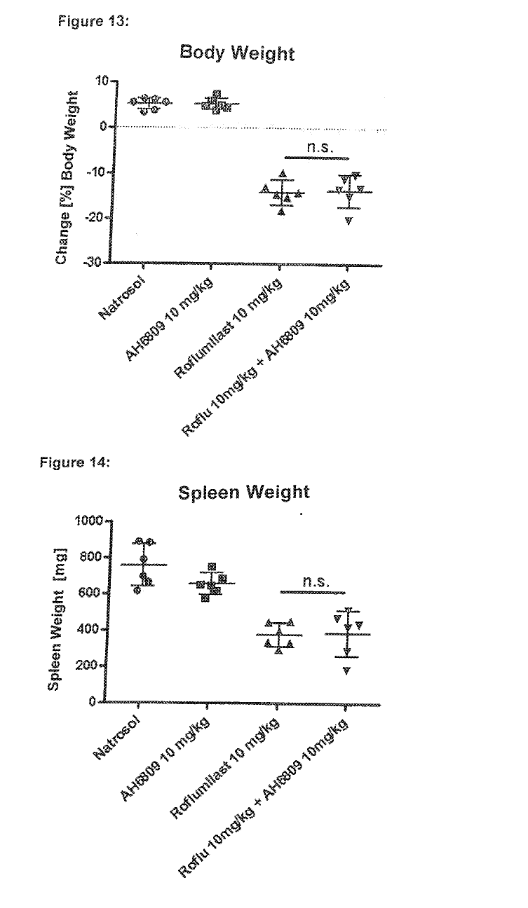

FIG. 13 shows the change in the body weight measured after the respective administrations in the control (Natrosol) group, the AH6809 group, the Roflumilast group and the Roflumilast+AH6809 group (statistics: Two-way analysis of variance; ns=not significant).

FIG. 14 shows the change in the spleen weight measured after the respective administrations in the control (Natrosol) group, the AH6809 group, the Roflumilast group and the Roflumilast+AH6809 group (statistics: Two-way analysis of variance; ns=not significant).

FIG. 15 shows the percentage of neutrophils in the blood after the respective administrations in the control (Natrosol) group, the AH6809 group, the Roflumilast group and the Roflumilast+AH6809 group (statistics: Two-way analysis of variance; ns=not significant).

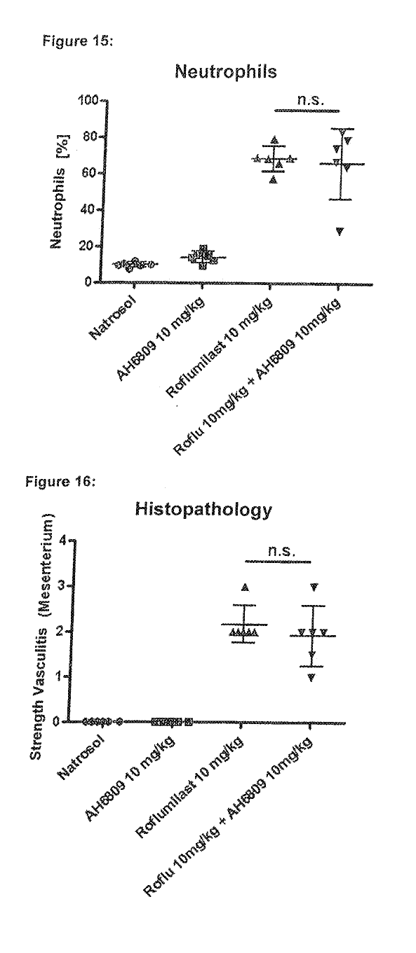

FIG. 16 shows the increase in the vasculitis observed in the mesentery after the respective administrations in the control (Natrosol) group, the AH6809 group, the Roflumilast group and the Roflumilast+AH6809 group (statistics: Two-way analysis of variance; ns=not significant).

To summarise, it can be stated that there were no indications that the EP2 preferential receptor antagonist AH-809 had any influence on the PDE4 inhibitor-mediated side effects observed in the Roflumilast group such as loss of body weight (FIG. 13), loss of spleen weight (FIG. 14), neutrophilia (FIG. 15) and mesenteric vasculitis (monocyte/polymorphonuclear perivascular infiltration in the mesentery as a measurement of vasculitis, FIG. 16). The parameters measured after the administration of AH6809 on its own appeared to be very similar to the control group.

All in all it can be concluded that the protective effect of an NSAID or a COX-2 inhibitor on the PDE4-inhibitor-mediated side effects is based at least partly on reducing the prostaglandin E2 synthesis of the COX-2 enzyme, which mediates the side effects through the EP4 receptor further on down the signal chain. Therefore the side effects can also be reduced by blocking the EP4 receptor. What is important in this context is that the EP4 receptor itself signals into the cell through an increase in the messenger molecule cAMP and cAMP is the substrate for the PDE4 enzymes that are inhibited by roflumilast. Therefore the influence of the PDE4 inhibitor is doubled, firstly resulting from the increase in the expression of cyclooxygenase-2, and concomitantly with that an increased production of prostaglandin E2 (PGE2) and at the same time an intensification of the PGE2/EP4-mediated signal (cAMP) in the affected cells as a result of the prevention of cAMP degradation. Blockage of the second cAMP-coupled prostaglandin E2 receptor EP2 by the EP2 preferential antagonist AH-6809 shows no effect on the parameters measured, in this context.

Thus, in the treatment of COPD and asthma patients with a PDE4 inhibitor, the simultaneous administration of an EP4 receptor antagonist such as MP498, for example, has major advantages, as on the one hand this EP4-receptor-antagonist can substantially reduce or even prevent the PDE4-receptor-mediated side effects and on the other hand, when an EP4 receptor antagonist is administered, in contrast to NSAID and COX-2 inhibitors, no appreciable side effects of its own are to be expected, even in long-term therapy.

### FORMULATIONS

The active substance combinations of 1 and 2 are preferably administered orally. For this purpose the ingredients (1) and (2) have to be presented in suitable oral preparations.

Suitable oral forms for administration are for example tablets, capsules, solutions, syrups or emulsions. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatine capsule), as a solution or suspension.

It is particularly preferable if the preparations are administered once or twice a day. Suitable tablets may be obtained, for example, by mixing the active substance(s) with known excipients, for example inert diluents such as calcium carbonate, calcium phosphate, microcrystalline cellulose, sorbitol, mannitol, isomaltose or lactose, disintegrants such as corn starch, crosslinked polyvinyl pyrrolidone, crosslinked sodium carboxymethylcellulose, sodium starch glycolate or alginic acid, binders such as starch, hydroxypropylmethylcellulose, polyvinylpyrrolidone or gelatine, lubricants, such as magnesium stearate or talc, and/or agents for delaying release, such as hydroxypropylcellulose, hydroxypropylmethylcellulose, ethylcellulose, aminomethacrylate, polyvinylpyrrolidone-polyvinylacetate copolymer, carboxymethylcellulose or polyvinylacetate. The tablets may also comprise several layers.

Coated tablets or film-coated tablets may be prepared accordingly by coating cores produced analogously to the tablets with substances normally used for tablet or film coatings, for example collidone or shellac, gum arabic, talc, titanium dioxide, sugar, hydroxypropylmethyl cellulose, ethycellulose, cellulose acetate phthalate, polymethacrylate, polyethyleneglycol, polyvinylalcohol, polyvinylalcohol-polyethyleneglycol copolymers or polyvinylacetate. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the above-mentioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

### Examples of Formulations

The following formulation examples for combined formulations are intended to serve to illustrate the invention without restricting it thereto. In particular, the active substances 1 and 2 may also be present in separate formulations and administered separately within a time window of not more than 6 hours.

| | | | |
|---|---|---|---|
| 1) | 0.10 | mg | active substance 1 |
| | 5.00 | mg | active substance 2.1 |
| | 25.00 | mg | lactose |
| | 202.40 | mg | microcrystalline cellulose |
| | 7.50 | mg | polyvinylpyrrolidone |
| | 7.50 | mg | crosslinked polyvinylpyrrolidone |
| | 2.50 | mg | magnesium stearate |
| | 250 | mg | |
| 2) | 0.10 | mg | active substance 1 |
| | 6.00 | mg | active substance 2.1 |
| | 25.00 | mg | lactose |
| | 201.40 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 3) | 0.10 | mg | active substance 1 |
| | 7.00 | mg | active substance 2.1 |
| | 25.00 | mg | lactose |
| | 200.40 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 4) | 0.10 | mg | active substance 1 |
| | 8.00 | mg | active substance 2.1 |
| | 25.00 | mg | lactose |
| | 199.40 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 5) | 0.10 | mg | active substance 1 |
| | 9.00 | mg | active substance 2.1 |
| | 25.00 | mg | lactose |
| | 198.40 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 6) | 0.50 | mg | active substance 1 |
| | 25.00 | mg | active substance 2.1 |
| | 25.00 | mg | lactose |
| | 182.00 | mg | microcrystalline cellulose |
| | 7.50 | mg | polyvinylpyrrolidone |
| | 7.50 | mg | crosslinked polyvinylpyrrolidone |
| | 2.50 | mg | magnesium stearate |
| | 250 | mg | |
| 7) | 0.50 | mg | active substance 1 |
| | 30.00 | mg | active substance 2.1 |
| | 25.00 | mg | lactose |
| | 177.00 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 8) | 0.50 | mg | active substance 1 |
| | 35.00 | mg | active substance 2.1 |
| | 25.00 | mg | lactose |
| | 172.00 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 9) | 0.50 | mg | active substance 1 |
| | 40.00 | mg | active substance 2.1 |
| | 25.00 | mg | lactose |
| | 167.00 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 10) | 0.50 | mg | active substance 1 |
| | 45.00 | mg | active substance 2.1 |
| | 25.00 | mg | lactose |
| | 162.00 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 11) | 1.00 | mg | active substance 1 |
| | 50.00 | mg | active substance 2.1 |
| | 25.00 | mg | lactose |
| | 156.50 | mg | microcrystalline cellulose |
| | 7.50 | mg | polyvinylpyrrolidone |
| | 7.50 | mg | crosslinked polyvinylpyrrolidone |
| | 2.50 | mg | magnesium stearate |
| | 250 | mg | |
| 12) | 1.00 | mg | active substance 1 |
| | 60.00 | mg | active substance 2.1 |
| | 25.00 | mg | lactose |
| | 146.50 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 13) | 1.00 | mg | active substance 1 |
| | 70.00 | mg | active substance 2.1 |
| | 25.00 | mg | lactose |
| | 136.50 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 14) | 1.00 | mg | active substance 1 |
| | 80.00 | mg | active substance 2.1 |
| | 25.00 | mg | lactose |
| | 126.50 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 15) | 1.00 | mg | active substance 1 |
| | 90.00 | mg | active substance 2.1 |
| | 25.00 | mg | lactose |
| | 116.50 | mg | microcrystalline cellulose |
| | 7.50 | mg | crosslinked polyvinylpyrrolidone |
| | 7.50 | mg | polyvinylpyrrolidone |
| | 2.50 | mg | magnesium stearate |
| | 250 | mg | |
| 16) | 5.00 | mg | active substance 1 |
| | 250.00 | mg | active substance 2.1 |
| | 50.00 | mg | lactose |
| | 160.00 | mg | microcrystalline cellulose |
| | 15.00 | mg | polyvinylpyrrolidone |
| | 15.00 | mg | crosslinked polyvinylpyrrolidone |
| | 5.00 | mg | magnesium stearate |
| | 500 | mg | |
| 17) | 5.00 | mg | active substance 1 |
| | 300.00 | mg | active substance 2.1 |
| | 50.00 | mg | lactose |
| | 110.00 | mg | microcrystalline cellulose |
| | 15.00 | mg | crosslinked polyvinylpyrrolidone |
| | 15.00 | mg | polyvinylpyrrolidone |
| | 5.00 | mg | magnesium stearate |
| | 500 | mg | |
| 18) | 5.00 | mg | active substance 1 |
| | 350.00 | mg | active substance 2.1 |
| | 50.00 | mg | lactose |
| | 60.00 | mg | microcrystalline cellulose |
| | 15.00 | mg | crosslinked polyvinylpyrrolidone |
| | 15.00 | mg | polyvinylpyrrolidone |
| | 5.00 | mg | magnesium stearate |
| | 500 | mg | |
| 19) | 5.00 | mg | active substance 1 |
| | 400.00 | mg | active substance 2.1 |
| | 60.00 | mg | lactose |
| | 93.00 | mg | microcrystalline cellulose |
| | 18.00 | mg | crosslinked polyvinylpyrrolidone |
| | 18.00 | mg | polyvinylpyrrolidone |
| | 6.00 | mg | magnesium stearate |
| | 600 | mg | |
| 20) | 5.00 | mg | active substance 1 |
| | 450.00 | mg | active substance 2.1 |
| | 60.00 | mg | lactose |
| | 43.00 | mg | microcrystalline cellulose |
| | 18.00 | mg | crosslinked polyvinylpyrrolidone |
| | 18.00 | mg | polyvinylpyrrolidone |
| | 6.00 | mg | magnesium stearate |
| | 600 | mg | |

1. A medicament comprising a PDE4-inhibitor and a EP4 receptor antagonist.

2. The medicament according to claim 1, wherein the EP4-receptor antagonist is an EP4-specific antagonist.

3. The medicament according to claim 1, wherein the EP4-receptor antagonist is:
[N-{[4-(5,9-diethoxy-6-oxo-6,8-dihydro-7H-pyrrolo[3,4-g]quinolin-7-yl)-3-methylbenzyl]sulphonyl}-2-(2-methoxyphenyl)acetamide] (2.1);
5-butyl-2,4-dihydro-4-[[2′-[N-(3-methyl-2-thiophene-carbonyl)sulphamoyl]biphenyl-4-yl]methyl]-2-[(2-trifluoromethyl)phenyl]-1,2,4-triazol-3-one (2.2);
(4-{(1S)-1-[({5-chloro-2-[(4-fluorophenyl)oxy]phenyl}carbonyl)amino]ethyl}benzoic acid (2.3);
N-[({2-[4-(2-ethyl-4,6-dimethyl-1H-imidazo[4,5-c]pyridin-1-yl)phenyl]ethyl}-amino)carbonyl]-4-methylbenzene sulphonamide (2.4);
4-[[4-(5-methoxy-2-pyridinyl)phenoxy]methyl]-5-methyl-N-[(2-methylphenyl)sulphonyl]-2-furancarboxamide (2.5);
methyl 11alpha,15alpha-dihydroxy-16-(3-methoxymethylphenyl)-9-oxo-17,18,19,20-tetranor-5-thia-13(E)prostanoate (2.6);
4-cyano-2-[[2-(4-fluoro-1-naphthalenyl)-1-oxopropyl]amino]-benzenebutanoic acid (2.7); or
N-{2-[4-(4,9-diethoxy-1-oxo-1,3-dihydro-2H-benzo[f]isoindol-2-yl)phenyl]acetyl}-benzene sulphonamide (2.8).

4. The medicament according to claim 1, the PDE4 inhibitor is a compound of formula 1 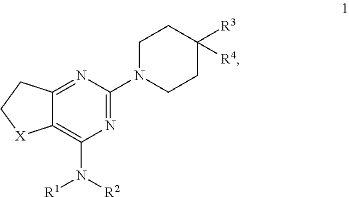
wherein:
X is SO or SO₂,
R¹ is H or C₁₋₆-alkyl,
R² is H or a group selected from C₁₋₁₀-alkyl and C₂₋₆-alkenyl, each optionally substituted by one or more groups selected from halogen and C₁₋₃-fluoroalkyl or which is optionally substituted by one or more groups selected from OR^{2.1}, COOR^{2.1}, CONR^{2.2}R^{2.3}, SR^{2.1}, SO—R^{2.1}, SO₂—R^{2.1}, C₆₋₁₀-aryl, het, hetaryl, a mono- or bicyclic C₃₋₁₀-cycloalkyl, CH₂—NR^{2.2}R^{2.3}, and NR^{2.2}R^{2.3}, each optionally substituted by one or more groups selected from OH, halogen, OR^{2.1}, oxo, CF₃, CHF₂, CH₂F, C₁₋₆-alkyl, C₁₋₆-alkanol, C₆₋₁₀-aryl, COOR^{2.1}, CH₂—NR^{2.2}R^{2.3}, and NR^{2.2}R^{2.3},
wherein
het is a three- to eleven-membered, mono- or bicyclic, saturated or partly saturated, optionally annellated or optionally bridged heterocyclic group, which contains 1, 2, 3, or 4 heteroatoms independently selected from N, S, or O,
hetaryl is a five- to ten-membered, mono- or bicyclic, optionally annellated heteroaryl, which contains 1, 2, 3, or 4 heteroatoms independently selected from N, S, or O, and
cycloalkyl is saturated or partly saturated,
wherein R^{2.1} is H or a group selected from C₁₋₆-alkyl, C₁₋₆-alkanol, C₁₋₃-haloalkyl, mono- or bicyclic, —C₃₋₁₀-cycloalkyl, C₆₋₁₀-aryl-C₁₋₆-alkylene, hetaryl-C₁₋₆-alkylene, het-C₁₋₆-alkylene, C₃₋₁₀-cycloalkyl-C₁₋₆-alkylene, a mono- or bicyclic C₆₋₁₀-aryl, heteroaryl, and a -het, each optionally substituted by one or more groups selected from OH, O—(C₁₋₃-alkyl), halogen, C₁₋₆-alkyl, and C₆₋₁₀-aryl,
wherein R^{2.2} and R^{2.3} are each independently H or a group selected from C₁₋₆-alkyl, mono- or bicyclic C₃₋₁₀cycloalkyl, C₆₋₁₀-aryl-C₁₋₆-alkylene, hetaryl-C₁₋₆-alkylene, mono- or bicyclic C₆₋₁₀-aryl, het, hetaryl, CO—NH₂, CO—NHCH₃, —CO—N(CH₃)₂, SO₂—(C₁-C₂-alkyl), CO—R^{2.1}, and COOR^{2.1}, each optionally substituted by one or more groups selected from OH, halogen, C₁₋₆-alkyl, C₆₋₁₀-aryl, and COOR^{2.1}, or
R² is a mono- or polycyclic C₃₋₁₀ cycloalkyl optionally singly or multiply bridged by C₁₋₃-alkyl groups and optionally substituted by a group selected from branched or unbranched C₁₋₆-alkanol, C₁₋₃-fluoroalkyl, C₁₋₃-alkylene-OR^{2.1}, OR^{2.1}, COOR^{2.1}, —SO₂—NR^{2.2}R^{2.3}, het, —NH—CO—O—(C₁₋₆-alkyl), —NH—CO—(C₁₋₆-alkyl), —NH—CO—O—(C₆₋₁₀-aryl), —NH—CO—(C₆₋₁₀-aryl), —NH—CO—O-hetaryl, —NH—CO-hetaryl, —NH—CO—O—(C₁₋₃-alkylene)-(C₆₋₁₀-aryl), —NH—CO—(C₁₋₃-alkylene)-(C₆₋₁₀-aryl), —N(C₁₋₃-alkyl)-CO—(C₁₋₆-alkyl), —N(C₁₋₃-alkyl)-CO—O—(C₆₋₁₀-aryl), —N(C₁₋₃-alkyl)-CO—(C₆₋₁₀-aryl), —N(C₁₋₃-alkyl)-CO—O-hetaryl, —N(C₁₋₃-alkyl)-CO-hetaryl, —N(C₁₋₃-alkyl)-CO—O—(C₁₋₃-alkylene)-(C₆₋₁₀-aryl), —N(C₁₋₃-alkyl)-CO—(C₁₋₃-alkylene)-(C₆₋₁₀-aryl), C₆₋₁₀-aryl, C₁₋₆-alkyl, C₆₋₁₀-aryl-C₁₋₆-alkylene, hetaryl-C₁₋₆-alkylene, mono- or bicyclic C₃₋₁₀ cycloalkyl and NR^{2.2}R^{2.3}, each optionally substituted by one or more groups selected from OH, OR^{2.1}, oxo, halogen, CF₃, CHF₂, CH₂F, C₁₋₆-alkyl, C₆₋₁₀-aryl, and NR^{2.2}R^{2.3}, or
R² is a mono- or polycyclic C₆₋₁₀-aryl optionally substituted by OH, SH, or halogen or by one or more groups selected from OR^{2.1}, COOR^{2.1}, NR^{2.2}R^{2.3}, CH₂—NR^{2.2}R^{2.3}, C₃₋₁₀-cycloalkyl, het, C₁₋₆-alkyl, C₁₋₃-fluoroalkyl, CF₃, CHF₂, CH₂F, C₆₋₁₀-aryl-C₁₋₆-alkylene, het-C₁₋₆-alkylene, hetaryl-C₁₋₆-alkylene, C₆₋₁₀-aryl, SO₂—CH₃, SO₂—CH₂CH₃ and SO₂—NR^{2.2}R^{2.3}, each optionally substituted by one or more or several groups selected from OH, OR^{2.1}, CF₃, CHF₂, CH₂F, oxo, halogen, CF₃, CHF₂, CH₂F, C₁₋₆-alkyl, C₆₋₁₀-aryl, and NR^{2.2}R^{2.3}, or
R² is a group selected from het and hetaryl, each optionally substituted by one or more groups selected from among halogen, OH, oxo, CF₃, CHF₂, and CH₂F or by one or more groups selected from OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, mono- or bicyclic C₃₋₁₀-cycloalkyl, C₆₋₁₀-aryl, C₁₋₆-alkyl, C₆₋₁₀-aryl-C₁₋₆-alkylene, hetaryl-C₁₋₆-alkylene, het, hetaryl, C₁₋₃-alkylene-OR^{2.1}, and NR^{2.2}R^{2.3}, each optionally sunstituted by one or more groups selected from OH, OR^{2.1}, oxo, halogen, CF₃, CHF₂, CH₂F, C₁₋₆-alkyl, C₆₋₁₀-aryl, and NR^{2.2}R^{2.3}, or
NR¹R² together are a heterocyclic C₄₋₇ ring that is optionally bridged, which contains 1, 2, or 3 heteroatoms selected from N, Q, and S, and optionally substituted by one or more groups selected from OH, OR^{2.1}, C₁₋₃-alkylene-O^{R.1}, oxo, halogen, C₁₋₆-alkyl, C₆₋₁₀-aryl, COOR^{2.1}, CH₂—NR^{2.2}—COO—R^{2.1}, CH₂—NR^{2.2}—CO—R^{2.1}, CH₂—NR^{2.2}—CO—CH₂—NR^{2.2}R^{2.3}, CH₂—NR^{2.2}—SO₂—C₁₋₃-alkyl, CH₂—NR^{2.2}—SO₂—NR^{2.2}R^{2.3}, CH₂—NR^{2.2}—CO—NR^{2.2}R^{2.3}, CO—NR^{2.2}R^{2.3}, CH₂—NR^{2.2}R^{2.3}, and NR^{2.2}R^{2.3}, and
R³ is a C₆₋₁₀-aryl optionally substituted in the ortho, para, or meta position with one, two, or three groups independently selected from fluorine, chlorine, bromine, hydroxy, CN, C₁₋₆-alkyl, C₁₋₃-fluoroalkyl, —C₁₋₃-alkylene-OR^{2.1}, —C₁₋₃-alkylene-NR^{2.2}R^{2.3}, —NR^{2.2}R^{2.3}, O—R^{2.1}; SO—R^{2.1}, SO₂—R^{2.1}, COOR^{2.1}, —CO—NH—(C₁₋₆-alkylene)-hetaryl, —CO—NH-hetaryl, —CO—N(CH₃)-het, —CO—N(CH₃)—(C₁₋₃-alkylene)-het, —CO—N(CH₃)—(C₁₋₃-alkylene)-hetaryl, —CO—N(C₃₋₇-cycloalkyl)-het, —CO—NR^{2.2}R^{2.3}, —CO—NH—(C₁₋₆-alkylene)-het, NR^{2.2}—CO—R^{2.1}, C₆₋₁₀-aryl, C₆₋₁₀-aryl-C₁₋₂-alkylene, het-C₁₋₂-alkylene, -het, —CO-het, —CO—N(CH₃)—C₃₋₇-cycloalkyl, C₃₋₇-cycloalkyl, C₃₋₇-cycloalkyl-C₁₋₂-alkylene, hetaryl-C₁₋₂-alkylene, and hetaryl, each optionally substituted by one or more groups selected from OH, halogen, —C₁₋₃-fluoroalkyl, oxo, methyl, and phenyl, or
R³ is a group selected from het and hetaryl, each optionally substituted by one or more groups selected from halogen, C₁₋₃-fluoroalkyl, CN, OH, oxo, —C₁₋₆-alkyl, —C₁₋₃-alkylene-NR^{2.2}R^{2.3}, —NR^{2.2}R^{2.3}, SO—R^{2.1}, SO₂—R^{2.1}, —O—R^{2.1}, —COOR^{2.1}, SO₂—(CH₃), SO₂—(CH₂—CH₃), C₆₋₁₀-aryl, het, C₃₋₇-cycloalkyl, and hetaryl, each optionally substituted by one or more groups selected from OH, halogen, —C₁₋₃-fluoroalkyl, C₁₋₆-alkyl, C₆₋₁₀-aryl, —COO(C₁₋₃-alkyl), and O—(C₁₋₃-alkyl), or
R³ is —O—R^{3.1}, wherein R^{3.1} is a group selected from C₁₋₆-alkyl, —C₆₋₁₀-aryl, —C₁₋₃-alkylene-C₆₋₁₀-aryl, hetaryl, and het, each optionally substituted in the ortho, para, or meta position by one, two, or three groups independently selected from fluorine, chlorine, bromine, hydroxy, CN, C₁₋₆-alkyl, C₁₋₃-fluoroalkyl, CO—(C₁₋₅-alkyl), —CO—(C₁₋₃-fluoroalkyl), —CO—NH—(C₁₋₆-alkylene)-hetaryl, —CO—N(C₁₋₃-alkyl)-(C₁₋₆-alkylene)-hetaryl, —CO—N(C₁₋₃-alkyl)-het, —CO—N(C₃₋₇-cycloalkyl)-het, —C₁₋₃-alkylene-OR^{2.1}, —C₁₋₃-alkylene-NR^{2.2}R^{2.3}, —NR^{2.2}R^{2.3}, O—R^{2.1}, SO—R^{2.1}, SO₂—R^{2.1}, COOH, COO—(C₁₋₄-alkyl), —O—C₁₋₃-alkylene-N(C₁₋₃-alkyl)₂, CO—NR^{2.2}R^{2.3}, NR^{2.2}—CO—R^{2.1}, C₆₋₁₀-aryl, C₆₋₁₀-aryl-C₁₋₂-alkylene, het-C₁₋₂-alkylene, —CO-het, het, —CO—C₃₋₇-cycloalkyl, —CO—N(C₁₋₃-alkyl)-C₃₋₇-cycloalkyl C₃₋₇-cycloalkyl, C₃₋₇-cycloalkyl-C₁₋₂-alkylene, hetaryl-C₁₋₂-alkylene, and hetaryl, each optionally substituted by 1, 2, 3, or 4 groups independently selected from F, Cl, Br, methyl, O-methyl, ethyl, O-ethyl, OH, oxo, and CF₃, and
R⁴ is H, CN, OH, CF₃, CHF₂, CH₂F, F, methyl, ethyl, —O—(C₁₋₃-alkyl), —C₁₋₃-alkylene-OH, —COO(C₁₋₃-alkyl), —CO-het, —(C₁₋₂-alkylene)-NH—SO₂—(C₁₋₂-alkyl), —(C₁₋₂-alkylene)-N(C₁₋₃-alkyl)-SO₂—(C₁₋₂-alkyl), —(C₁₋₂-alkylene)-O—(C₁₋₂-alkylene)-C₆₋₁₀-aryl, —C₁₋₃-alkylene-O—C₁₋₃-alkyl, —(C₁₋₂-alkylene)-N(C₁₋₃-alkyl)-CO—(C₁₋₂-alkyl), —NH—CO—(C₁₋₃-alkylene)-O—(C₁₋₃-alkyl), —C₁₋₃-alkylene-NH—CO—(C₁₋₃-alkyl), —C₁₋₃-alkylene-NH—CO—(C₁₋₃-alkylene)-N(C₁₋₃-alkyl)₂, —O—(C₁₋₂-alkylene)-(C₆₋₁₀-aryl), —C₁₋₃-alkylene-NH—CO—(C₁₋₃-alkylene)-O—(C₁₋₃-alkyl), —CO—(C₆₋₁₀-aryl), and —(C₁₋₂-alkylene)-N(C₁₋₃-alkyl)-CO—(C₁₋₂-alkylene)-O—(C₁₋₃-alkyl), wherein the aryl in the above groups are optionally substituted by one or more groups selected from F, Cl, Br, methyl, ethyl, propyl, isopropyl, cyclopropyl, —O-methyl, —O-ethyl, —O-propyl, —O-isopropyl, —O-cyclopropyl, —OH, and CF₃, or
R³ and R⁴ together form a mono- or bicyclic, unsaturated, saturated, or partly saturated heterocyclic group, which contains 1, 2, or 3 heteroatoms selected from N, O, and S, and optionally substituted by one or more groups selected from halogen, OH, oxo, C₁₋₃-fluoroalkyl, CN, C₁₋₆-alkyl, —O—R^{2.1}, —COOR^{2.1}, SO—R^{2.1}, SO₂—R^{2.1}, —C₁₋₃-alkylene-NR^{2.2}R^{2.3}, —NR^{2.2}R^{2.3}, C₆₋₁₀-aryl, C₃₋₇-cycloalkyl, het, and hetaryl.

5. The medicament according to claim 1, wherein:
X is SO,
R¹ is H,
R² is H or C₁₋₆-alkyl, optionally substituted by one or more groups selected from F, Cl, CF₃, CHF₂, or CH₂F or optionally substituted by one or more groups selected from OR^{2.1}, COOR^{2.1}, CONR^{2.2}R^{2.3}, SR^{2.1}, SO—R^{2.1}, SO₂—R^{2.1}, phenyl, het, hetaryl, a monocyclic C₃₋₇ cycloalkyl, CH₂—NR^{2.2}R^{2.3}, and NR^{2.2}R^{2.3}, each optionally substituted by one or more groups selected from OH, F, Cl, Br, CF₃, CHF₂, CH₂F, OR^{2.1}, oxo, methyl, ethyl, propyl, isopropyl, methanol, ethanol, phenyl, COOR^{2.1}, CH₂—NR^{2.2}R^{2.3}, and NR^{2.2}R^{2.3},
wherein
het is a three- to seven-membered, monocyclic, saturated or partly saturated heterocyclic group or a seven- to eleven-membered, bicyclic, saturated or partly saturated heterocyclic group which contains 1, 2, or 3 heteroatoms independently selected from N, S, or O,
hetaryl is a five- to six-membered, monocyclic, aromatic heteroaryl, or a seven- to eleven-membered, bicyclic, aromatic heteroaryl which contains 1, 2, or 3 heteroatoms independently selected from N, S, or O, and
cycloalkyl is saturated or partly saturated,
wherein R^{2.1} is H or a group selected from methyl, ethyl, propyl, isopropyl, methanol, ethanol, monocyclic C₃₋₇ cycloalkyl, phenyl-C₁₋₂-alkylene, -hetaryl-C₁₋₂-alkylene, -het-C₁₋₂-alkylene, C₃₋₇-cycloalkyl-C₁₋₂-alkylene, phenyl, hetaryl, and a het, each optionally substituted by one or more groups selected from OH, F, Cl, methyl, ethyl, propyl, isopropyl, O-methyl, O-ethyl, O-propyl, O-isopropyl, and phenyl,
wherein R^{2.2} and R^{2.3} are each independently H or a group selected from methyl, ethyl, propyl, isopropyl, monocyclic C₃₋₇cycloalkyl, phenyl-C₁₋₃-alkylene, hetaryl-C₁₋₃-alkylene, phenyl, -het, -hetaryl, CO—NH₂, CO—NHCH₃, CON(CH₃)₂, SO₂—(C₁₋₂-alkyl), CO—R^{2.1}, and COOR^{2.1}, each optionally substituted by one or more groups selected from OH, F, Cl, methyl, ethyl, propyl, isopropyl, phenyl, and COOR^{2.1}, or
R² is a monocyclic C₃₋₇ cycloalkyl optionally substituted by a group selected from C₁₋₂-alkanol, C₁₋₃-fluoroalkyl, C₁₋₃-alkylene-OR^{2.1}, OR^{2.1}, COOR^{2.1}, SO₂—NR^{2.2}R^{2.3}, -het, —NH—CO—O-(phenyl), methyl, ethyl, propyl, isopropyl, phenyl, phenyl-C₁₋₂-alkylene, -hetaryl-C₁₋₂-alkylene, monocyclic C₃₋₇ cycloalkyl, and NR^{2.2}R^{2.3}, each optionally substituted by one or more groups selected from OH, OR^{2.1}, oxo, F, Cl, CF₃, CHF₂, CH₂F, methyl, ethyl, propyl, isopropyl, phenyl, and NR^{2.2}R^{2.3}, or
R² is a phenyl optionally substituted by OH, SH, F, Cl, or Br or by one or more groups selected from OR^{2.1}, COOR^{2.1}, NR^{2.2}R^{2.3}, CH₂—NR^{2.2}R^{2.3}, monocyclic C₃₋₇-cycloalkyl, -het, methyl, ethyl, propyl, isopropyl, CF₃, CHF₂, CH₂F, phenyl-C₁₋₂-alkylene, het-C₁₋₂-alkylene, hetaryl-C₁₋₂-alkylene, phenyl, SO₂—CH₃, SO₂—CH₂CH₃, and SO₂—NR^{2.2}R^{2.3}, each optionally substituted by one or more groups selected from OH, OR^{2.1}, oxo, F, Cl, CF₃, CHF₂, CH₂F, methyl, ethyl, propyl, isopropyl, phenyl, and NR^{2.2}R^{2.3}, or
R² is a group selected from het and hetaryl, each optionally substituted by one or more groups selected from F, Cl, OH, oxo, CF₃, CHF₂, and CH₂F or by one or more groups selected from OR^{2.1}, C₁₋₃-alkylene-OR^{2.1}, SR^{2.1}, SO—R^{2.1}, SO₂—R^{2.1}, COOR^{2.1}, COR^{2.1}, methanol, ethanol, monocyclic C₃₋₇-cycloalkyl, phenyl, methyl, ethyl, propyl, isopropyl, phenyl-C₁₋₂-alkylene, hetaryl-C₁₋₂-alkylene, -het, -hetaryl, and NR^{2.2}R^{2.3}, each optionally substituted by one or more groups selected from OH, OR^{2.1}, oxo, F, Cl, CF₃, CHF₂, CH₂F, methyl, ethyl, propyl, isopropyl, butyl, isobutyl, phenyl, and NR^{2.2}R^{2.3}, and
R³ is a naphthalene or phenyl, each optionally substituted in the ortho, para, or meta position by one or two groups selected independently from fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO—CH₃, COOCH₃, COOCH₂CH₃, —CO—NH-(methylene)-hetaryl, —CO—NH-(ethylene)-hetaryl, —CO—NH-hetaryl, —CO—N(CH₃)-het, —CO—N(CH₃)-(methylene)-het, —CO—N(CH₃)-(ethylene)-het, —CO—N(CH₃)-(methylene)-hetaryl, —CO—N(CH₃)-(ethylene)-hetaryl, —CO—N(cyclopropyl)-het, CO—NH₂, CONH(CH₃), CON(CH₃)₂, —CO—NH-(methylene)-het, —CO—NH-(ethylene)-het, —NH—CO-methyl, NCH₃—CO-methyl, —NH—CO-ethyl, NCH₃—CO-ethyl, —NH—CO-propyl, NCH₃—CO-propyl, —NH—CO-isopropyl, NCH₃—CO-isopropyl, phenyl, phenyl-methylene, phenyl-ethylene, het-methylene, het-ethylene, -het, —CO-het, —CO—N(CH₃)-het, CO—N(CH₃)-cyclopropyl, C₃₋₇-cycloalkyl, C₃₋₇-cycloalkyl-methylene, C₃₋₇-cycloalkyl-ethylene, hetaryl-methylene, hetaryl-ethylene, -hetaryl, CH₂—NH₂, CH₂—NH(CH₃), CH₂—N(CH₃)₂, —NH₂, —NH(CH₃), and —N(CH₃)₂, each optionally substituted by one or more groups selected from among OH, F, Cl, —CF₃, CHF₂, CH₂F, oxo, methyl, and phenyl, or
R³ is a group selected from a het and hetaryl, each optionally substituted by one or more groups selected from F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO—(CH₃), SO—(CH₂—CH₃), SO₂—(CH₃), SO₂—(CH₂—CH₃), phenyl, CH₂—NH₂, CH₂—NH(CH₃), CH₂—N(CH₃)₂, —NH₂, —NH(CH₃), —N(CH₃)₂, het, and hetaryl, each optionally substituted by one or more groups selected from OH, F, Cl, CF₃, CHF₂, CH₂F, methyl, ethyl, propyl, isopropyl, phenyl, —COO-methyl, —COO-ethyl, O-methyl, and O-ethyl, or
R³ is —O—R^{3.1}, wherein R^{3.1} is a group selected from C₁₋₃-alkyl, -phenyl, —C₁₋₃-alkylene-phenyl, hetaryl, and het, each optionally substituted in the ortho, para, or meta position by one, two, or three groups selected independently from fluorine, chlorine, bromine, hydroxy, CN, methyl, ethyl, propyl, isopropyl, butyl, isobutyl, CF₃, CHF₂, CH₂F, CO-(methyl), CO-(ethyl), CO-(propyl), CO-(isopropyl), —CO—(CF₃), —CO—NH-(methylene)-hetaryl, —CO—NH-(ethylene)-hetaryl, —CO—N(CH₃)-(methylene)-hetaryl, —CO—N(CH₃)-(ethylene)-hetaryl, —CO—N(CH₃)-(propylene)-hetaryl, —CO—N(CH₃)-(isopropylene)-hetaryl-CO—N(CH₃)-het, —CO—N(cyclopropyl)-het, —CO—N(C₅₋₇-cycloalkyl)-het, -methylene-O-methyl, -ethylene-O-methyl, -propylene-O-methyl, -methylene-O-ethyl, -ethylene-O-ethyl, -propylene-O-ethyl, -methylene-NH₂, -methylene-NHCH₃, -methylene-N(CH₃)₂, -ethylene-NH₂, -ethylene-NHCH₃, -ethylene-N(CH₃)₂, NH₂, N(CH₃)₂, NHCH₃, —O-methyl, O-ethyl, O-propyl, O-isopropyl, O-butyl, O-isobutyl, —SO—CH₃, SO-ethyl, —SO-propyl, —SO-isopropyl,, SO₂-methyl, —SO₂-ethyl, SO₂-propyl, SO₂-isopropyl, COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NH(CH₃), CO—N(CH₃)₂, —NH—CO-methyl, —NCH₃—CO-methyl, —NH—CO-ethyl, NCH₃—CO-ethyl, phenyl, phenyl-methylene, phenyl-ethylene, het-methylene, het-ethylene, —CO-het, het, —CO—C₅₋₇-cycloalkyl, —CO-cyclopropyl, —CO—N(CH₃)—C₅₋₇-cycloalkyl, —CO—N(CH₃)-cyclopropyl, C₅₋₇-cycloalkyl, cyclopropyl, C₅₋₇-cycloalkyl-methylene, C₅₋₇-cycloalkyl-ethylene, cyclopropyl-methylene, cyclopropyl-ethylene, hetaryl-methylene, hetaryl-ethylene, and hetaryl, each optionally substituted by 1, 2, 3, or 4 groups independently selected from F, Cl, Br, methyl, O-methyl, ethyl, O-ethyl, OH, oxo, and CF₃, and
R⁴ is H, CN, OH, CF₃, CHF₂, CH₂F, F, methyl, ethyl, O-methyl or O-ethyl, -methylene-OH, -ethylene-OH, -propylene-OH, isopropylene-OH, —COO(methyl), —COO(ethyl), —COO(propyl), —COO(isopropyl), —CO-het, -(methylene)-NH—SO₂-(methyl), -(methylene)-NH—SO₂-(ethyl), -(ethylene)-NH—SO₂-(methyl), -(ethylene)-NH—SO₂-(ethyl), -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl, -ethylene-O-ethyl, -(methylene)-N(CH₃)—CO-(methyl), -(methylene)-N(CH₃)—CO-(ethyl)-(ethylene)-N(CH₃)—CO-(methyl), -(ethylene)-N(CH₃)—CO-(ethyl), —NH—CO -(methylene)-O-(methyl), —NH—CO-(methylene)-O-(ethyl), —NH—CO-(ethylene)-O -(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene)-N(CH₃)—CO-(ethylene)-O-(ethyl), -(ethylene)-N(CH₃)—CO -(methylene)-O-(ethyl), —O-(methylene)-phenyl, —O-(ethylene)-phenyl, or —CO-phenyl, wherein the phenyl in the above groups is optionally substituted by one or more other groups selected from F, Cl, Br, methyl, ethyl, propyl, —O-methyl, —O-ethyl, —O-propyl, —OH, and CF₃, or
R³ and R⁴ together form a mono- or bicyclic, unsaturated, saturated, or partly saturated heterocyclic group, which contains 1, 2, or 3 heteroatoms selected from N, O, and S and which is optionally substituted by one or more groups selected from F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂CH₃), SO—(CH₃), SO—(CH₂CH₃), CH₂—NH₂, CH₂—NH(CH₃), CH₂—N(CH₃)₂, —NH₂, —NH(CH₃), —N(CH₃)₂, phenyl, C₅₋₇-cycloalkyl, het, and hetaryl.

6. The medicament according to claim 1, wherein:
R² is a group of formula 3 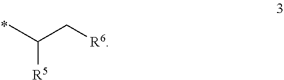
R⁶ is OH or NH₂, and
R⁵ is a group selected from C₁₋₄-alkyl, a five- to six-membered heteroaryl with 1, 2, or 3 heteroatoms selected from S, O, and N, and phenyl, each optionally substituted by one or more groups selected from OH, F, Br, OR^{2.1}, oxo, methyl, ethyl, methanol, ethanol, phenyl, COOR^{2.1}, CH₂—NR^{2.2}R^{2.3}, and NR^{2.2}R^{2.3}.

7. The medicament according to claim 6, wherein:
R⁵ is methyl, ethyl, propyl, or isopropyl.

8. The medicament according to claim 1, wherein:
R² is a monocyclic three, four, five, six, or seven-membered cycloalkyl ring optionally substituted in the spiro position by a group selected from —CH₂—OR^{2.1}, branched or unbranched C₂₋₆-alkylene-OR^{2.1}, methyl, ethyl, propyl, isopropyl, butyl, isobutyl, cyclopropyl, —CF₃, CHF₂, CH₂F, and C₂₋₄-fluoroalkyl, and wherein R^{2.1} is selected from methyl, ethyl, propyl, isopropyl, butyl, and isobutyl.

9. The medicament according to claim 1, wherein:
R² is a cyclopropyl optionally substituted by another group selected from —NH₂, CH₂—NH₂, —NH(CH₃), —N(CH₃)₂, methyl, ethyl, propyl, isopropyl, —NH—CO-(tert-butyl), —NH—CO—O-(tert-butyl), —N(CH₃)—CO-(tert-butyl), —N(CH₃)—CO—O-(tert-butyl), —CF₃, —CHF₂, CH₂F, F, Cl, and Br.

10. The medicament according to claim 1, wherein:
R² is a phenyl optionally substituted in one or both meta positions by one or more groups selected from methyl, ethyl, propyl, isopropyl, cyclopropyl, F, Cl, Br, OH, OR^{2.1}, COOR^{2.1}, CF₃, CHF₂, CH₂F, NH₂, NH(CH₃), and N(CH₃)₂, wherein R^{2.1}— is H, methyl, or ethyl.

11. The medicament according to claim 1, wherein:
R² is a monocyclic, saturated three, four, five, six, or seven-membered heterocyclic group with 1, 2, or 3 heteroatoms selected in each case from N, O, and S, optionally substituted by one or more groups selected from fluorine, chlorine, bromine, CF₃, CHF₂, CH₂F, OH, and oxo or by one or more groups selected from OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, C₃₋₁₀-cycloalkyl, phenyl, C₁₋₆-alkyl, phenyl-C₁₋₆-alkylene, hetaryl-C₁₋₆-alkylene, het, hetaryl, and NR^{2.2}R^{2.3}, each optionally substituted by one or more groups selected from OH, OR^{2.1}, oxo, F, Cl, CF₃, CHF₂, CH₂F, C₁₋₆-alkyl, phenyl, and NR^{2.2}R^{2.3}.

12. The medicament according to claim 11, wherein:
R² is a monocyclic, saturated six-membered heterocyclic group with a heteroatom selected from N, O, and S, optionally substituted by one or more groups selected from F, Cl, Br, CF₃, CHF₂, CH₂F, OH, oxo, NH₂, NHCH₃, N(CH₃)₂, methyl, ethyl, propyl, isopropyl, cyclopropyl, methoxy, and ethoxy.

13. The medicament according to claim 1, wherein:
R² is a group selected from piperidine or tetrahydropyran, each optionally substituted by one or more groups selected from F, Cl, Br, OH, CF₃, CHF₂, CH₂F, NH₂, NHCH₃, N(CH₃)₂, oxo, methyl, and methoxy.

14. The medicament according to claim 1, wherein:
R³ is a naphthalene or phenyl, each optionally be substituted in any position by one, two, or three groups independently selected from fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO₂—CH₂CH₃, COOCH₃, and CO—O—CH₂CH₃—.

15. The medicament according to claim 1, wherein:
R³ is a group selected from het and hetaryl, each optionally substituted by one or more groups selected from F, Cl, Br, CF₃, CHF₂, CH₂F, CN, OH, oxo, methyl, ethyl, propyl, isopropyl, butyl, isobutyl, cyclopropyl, C₅₋₇-cycloalkyl, —O-methyl, —O-ethyl, —O-propyl, —O-isopropyl, —COO-methyl, —COO-ethyl, —COO-propyl, —COO-isopropyl, SO₂—(CH₃), SO₂—(CH₂—CH₃), SO—(CH₃), SO—(CH₂—CH₃), phenyl, —CH₂—NH₂, —CH₂—NHCH₃, —CH₂—N(CH₃)₂, NH₂, NHCH₃, N(CH₃)₂, het, and hetaryl, each optionally substituted by one or more groups selected from OH, F, Cl, Br, CF₃, CHF₂, CH₂F, methyl, ethyl, propyl, isopropyl, phenyl, —COO-methyl, —COO-ethyl, —COO-propyl, —COO-isopropyl, O-methyl, O-ethyl, O-propyl, and O-isopropyl, and
R⁴ is H, CN, OH, CF₃, CHF₂, CH₂F, F, methyl, ethyl, O-methyl, or O-ethyl, and
wherein
het is a three- to seven-membered, monocyclic, saturated or partly saturated heterocyclic group or a seven- to eleven-membered, bicyclic, annellated, saturated, or partly saturated heterocyclic group, which contains 1, 2, or 3 heteroatoms independently selected from N, S, or O,
hetaryl is a five- to six-membered, monocyclic, aromatic heteroaryl or a seven- to eleven-membered, bicyclic, annellated, aromatic heteroaryl which contains in each case 1, 2, or 3 heteroatoms heteroatoms independently selected from N, S, or O, and
cycloalkyl is saturated or partly saturated,

16. The medicament according to claim 1, wherein:
R³ is indole, dihydroindole, quinazoline, dihydroquinazoline, tetrahydroquinazoline, benzoisoxazole, dihydrobenzoisoxazole, benzoxazine, dihydrobenzoxazine, benzothiazole, dihydrobenzothiazole, triazolopyridine, dihydrotriazolopyridine, benzofuran, dihydrobenzofuran, isobenzofuran, and dihydroisobenzofuran, each optionally substituted by one or more groups selected from F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO₂—(CH₃), SO₂—(CH₂—CH₃), SO—(CH₃), SO—(CH₂—CH₃), phenyl, —CH₂—NH₂, —CH₂—NHCH₃, —CH₂—N(CH₃)₂, NH₂, NHCH₃, N(CH₃)₂, furanyl, and pyridinyl, each optionally substituted by one or more groups selected from OH, F, Cl, Br, CF₃, CHF₂, CH₂F, methyl, ethyl, propyl, isopropyl, phenyl, —COO-methyl, —COO-ethyl, O-methyl, and O-ethyl,

17. The medicament according to claim 1, wherein:
R³ is imidazole, dihydroimidazole, oxadiazole, oxadiazolidine, pyrazole, pyridine, and dihydropyrazole, each optionally substituted by one or more groups selected from F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO₂—(CH₃), SO₂—(CH₂—CH₃), SO—(CH₃), SO—(CH₂—CH₃), phenyl, —CH₂—NH₂, —CH₂—NHCH₃, —CH₂—N(CH₃)₂, NH₂, NHCH₃, N(CH₃)₂, furanyl, and pyridinyl, each optionally substituted by one or more groups selected from OH, F, Cl, Br, CF₃, CHF₂, CH₂F, methyl, ethyl, propyl, isopropyl, phenyl, —COO-methyl, —COO-ethyl; O-methyl, and O-ethyl,

18. The medicament according to claim 1, wherein:
R³ and R⁴ together form a mono- or bicyclic, unsaturated or partly saturated, three- to eleven-membered heterocyclic group which contains 1, 2, or 3 heteroatoms selected from N, O, and S and optionally substituted by one or more groups selected from F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂—CH₃), SO—(CH₃), SO—(CH₂—CH₃), phenyl, —CH₂—NH₂, —CH₂NHCH₃, —CH₂—N(CH₃)₂, NH₂, NHCH₃, N(CH₃)₂, a saturated or partly saturated, five- to six-membered heterocyclic group, and a five- to six-membered heteroaryl.

19. The medicament according to claim 18, wherein:
R³ and R⁴ together form a bicyclic heterocyclic group selected from tetrahydroquinazoline, tetrahydrobenzoxazin, dihydroindole, and dihydroisobenzofuran each optionally substituted by one or more groups selected from F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂—CH₃), phenyl, —CH₂—NH₂, —CH₂NHCH₃, —CH₂—N(CH₃)₂, NH₂, NHCH₃, N(CH₃)₂, a saturated or partly saturated, five- or six-membered heterocyclic group, and a five- or six-membered heteroaryl.

20. The medicament according to claim 1, wherein:
R³ is —O—R^{3.1}, wherein R^{3.1} is a group selected from methyl, ethyl, propyl, isopropyl, butyl, isobutyl, pentyl, isopentyl, -phenyl, -methylene-phenyl, -ethylene-phenyl, -propylene-phenyl, -isopropylene-phenyl, hetaryl, and het, each optionally substituted in the ortho, para, or meta position by one, two, or three groups independently selected from fluorine, chlorine, bromine, hydroxy, CN, methyl, ethyl, propyl, isopropyl, butyl, isobutyl, —CF₃, CHF₂, CH₂F, CO-(methyl), CO-(ethyl), CO-(propyl), CO-(isopropyl), CO-(butyl), CO-(isobutyl), —CO—(CF₃), —CO—(CH₂F), —CO—(CHF₂), —CO—NH-(methylene)-hetaryl, —CO—NH-(ethylene)-hetaryl, —CO—NH-(propylene)-hetaryl, —CO—NH-(isopropylene)-hetaryl, —CO—N(CH₃)-(methylene)-hetaryl, —CO—N(CH₃)-(ethylene)-hetaryl, —CO—N(CH₃)-(propylene)-hetaryl, —CO—N(CH₃)-(isopropylene)-hetaryl, —CO—N(CH₃)-het, —CO—N(C₃₋₇-cycloalkyl)-het, -methylene-O-methyl, -ethylene-O-methyl, -methylene-O-ethyl, -ethylene-O-ethyl, -methylene-NH₂, -ethylene-NH₂, -methylene-NHCH₃, -ethylene-NHCH₃, -methylene-N(CH₃)₂, -ethylene-N(CH₃)₂, —NH₂, —NHCH₃, —N(CH₃)₂, —O-methyl, —O-ethyl, —O-propyl, —O-isopropyl, —SO—CH₃, —SO—(CH₂CH₃), —SO₂—CH₃, —SO₂—(CH₂CH₃), COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NHCH₃, CO—N(CH₃)₂, NH—CO-methyl, NCH₃—CO-methyl, NH—CO-ethyl, N(CH₃)—CO-ethyl, phenyl, phenyl-methylene, phenyl-ethylene, het-methylene, het-ethylene, —CO-het, het, —CO—C₄₋₇-cycloalkyl, —CO-cyclopropyl, —CO—N(CH₃)-cyclopropyl, —CO—N(CH₃)—C₄₋₇-cycloalkyl, C₄₋₇-cycloalkyl, cyclopropyl, C₄₋₇-cycloalkyl-methylene, cyclopropyl-methylene, C₄₋₇-cycloalkyl-ethylene, cyclopropyl-ethylene, hetaryl-methylene, hetaryl-ethylene-, and hetaryl, each optionally substituted by 1, 2, 3, or 4 groups independently selected from F, Cl, Br, methyl, O-methyl, ethyl, O-ethyl, OH, oxo, and CF₃,

21. The medicament according to claim 1, wherein:
R⁴ is H, CN, OH, CF₃, CHF₂, CH₂F, F, methyl, ethyl, O-methyl or O-ethyl, -methylene-OH, -ethylene-OH, -propylene-OH, isopropylene-OH, —COO(methyl), —COO(ethyl), —COO(propyl), —COO(isopropyl), —CO-het, -(methylene)-NH—SO₂-(methyl), -(methylene)-NH—SO₂-(ethyl), -(ethylene)-NH—SO₂-(methyl), -(ethylene)-NH—SO₂-(ethyl), -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl, -ethylene-O-ethyl, -(methylene)-N(CH₃)—CO-(methyl), -(methylene)-N(CH₃)—CO-(ethyl), -(ethylene)-N(CH₃)—CO-(methyl), -(ethylene)-N(CH₃)—CO-(ethyl), —NH—CO -(methylene)-O-(methyl), —NH—CO-(methylene)-O-(ethyl), —NH—CO-(ethylene)-O -(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene)-N(CH₃)—CO-(ethylene)-O-(ethyl), -(ethylene)-N(CH₃)—CO -(methylene)-O-(ethyl), —O-(methylene)-phenyl, —O-(ethylene)-phenyl, and —CO-phenyl, and wherein the phenyl in the above groups are optionally substituted by one or more other groups selected from F, Cl, Br, methyl, ethyl, propyl, —O-methyl, —O-ethyl, —O-propyl, —OH, and CF₃,

22. The medicament according to claim 1, wherein:
R³ is a group selected from oxazole, imidazole and thiazole, each optionally substituted by one, two, or three further groups independently selected from methyl, ethyl, propyl, isopropyl, O-methyl, O-ethyl, O-propyl, O-isopropyl, OH, F, Cl, Br, CF₃, phenyl, hetaryl, and C₃₋₆-cycloalkyl,

23. The medicament according to claim 1, wherein X is SO₂.

24. The medicament according to claim 1, wherein the PDE4 inhibitor is:
1.1. (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol;
1.2. (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol;
1.3. (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol;
1.4. (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol;
1.5. (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one;
1.6. {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine;
1.7. 1-(4-(1-hydroxymethylcyclopropylamino)-5-oxo-6,7-dihydro-5H-5λ⁴-thieno[3,2-d]pyrimidin-2-yl)-3′-methyl-1′H-spiro[piperidin-4,4′-quinazolin]-2′(3′H)-one;
1.8. {1-[2-(4-benzo[d]isoxazol-3-yl-piperidin-1-yl)-5-oxo-6,7-dihydro-5H-5λ⁴-thieno[3,2-d]pyrimidin-4-ylamino]-cyclopropyl}-methanol;
1.9. (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol;
1.10. 1-[4-((S)-1-methyl-6-oxopiperidin-3-ylamino)-5-oxo-6,7-dihydro-5H-5λ⁴-thieno[3,2-d]pyrimidin-2-yl]-4-phenylpiperidin-4-carbonitrile;
1.11. 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(3′H)-one;
1.12. (3-fluorophenyl)-[5-oxo-2-(3,4,5,6-tetrahydro-2H-[4,4]bipyridinyl-1-yl)-6,7-dihydro-5H-5λ⁴-thieno[3,2-d]pyrimidin-4-yl]-amine;
1.13. {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine;
1.14. (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol;
1.15. {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine;
1.16. (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one;
1.17. (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol;
1.18. (1-{2-[4-(5-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol;
1.19. {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine;
1.20. (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine;
1.21. (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol;
1.22. (S)-5-{2-[4-(4-fluorophenoxy)-piperidin-1-yl]-5-oxo-6,7-dihydro-5H-5λ⁴-thieno[3,2-d]pyrimidin-4-ylamino}-1-methylpiperidin-2-one;
1.23. (2-{4-[4-(4,5-dihydroxazol-2-yl)-phenoxy]-piperidin-1-yl}-5-oxo-6,7-dihydro-5H-5λ⁴-thieno[3,2-d]pyrimidin-4-yl)-(tetrahydropyran-4-yl)-amine;
1.24. 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid;
1.25. 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol;
1.26. {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine;
1.27. 2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl)-(tetrahydropyran-4-yl)-amine;
1.28. (S)-5-(2-{4-[4-(4,5-dihydroxazol-2-yl)-phenoxy]-piperidin-1-yl}-5-oxo-6,7-dihydro-5H-5λ⁴-thieno[3,2-d]pyrimidin-4-ylamino)-1-methylpiperidin-2-one;
1.29. {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine;
1.30. {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine;
1.31. 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide;
1.32. N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide;
1.33. N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yloxy}-benzamide;
1.34. {5-oxo-2-[4-(pyridin-4-yloxy)-piperidin-1-yl]-6,7-dihydro-5H-5λ⁴-thieno[3,2-d]pyrimidin-4-yl}-(tetrahydropyran-4-yl)-amine;
1.35. {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine;
1.36. (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one;
1.37. (1-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-cyclopropyl)-methanol;
1.38. (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one;
1.39. {4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-yl}-methanol;
1.40. [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol;
1.41. 4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-ol;
1.42. {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine;
1.43. 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile;
1.44. 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl}-(tetrahydropyran-4-yl)-amine; or
1.45. (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one.

25. The medicament according to claim 1, wherein the PDE4 inhibitor is contained in a daily dose of 0.01 mg to 50 mg.

26. The medicament according to claim 1, wherein the EP4-receptor antagonist is used in a daily dose of 0.001 to 100 mg/kg body weight.

27. The medicament according to claim 1, wherein the EP4-receptor antagonist and the PDE4 inhibitor are used in a ratio by weight of 1:1 to 200:1.

28.-40. (canceled)
